(19) United States
(12) Patent Application Publication
(10) Pub. No.: US 2022/0144881 A1
(43) Pub. Date: May 12, 2022

(54) COMPOUNDS AND METHODS FOR MANUFACTURE OF HYPOMETHYLATING AGENTS

(71) Applicant: Astex Pharmaceuticals, Inc., Pleasanton, CA (US)

(72) Inventors: Masahiro MIYAKE, Tokushima (JP); Masahiro SOTA, Tokushima (JP); Shin OGASAWARA, Tokushima (JP); Nao KOSEKI, Tokushima (JP); Neil G. ANDERSEN, Montara, CA (US); Ramakrishnan CHIDAMBARAM, Dublin, CA (US); Nipun DAVAR, Pleasanton, CA (US)

(21) Appl. No.: 17/319,714

(22) Filed: May 13, 2021

Related U.S. Application Data
(60) Provisional application No. 63/112,429, filed on Nov. 11, 2020.

(51) Int. Cl. C07H 19/20

(52) U.S. Cl. CPC C07H 19/20; C07B 2200/13

(57) ABSTRACT

The present disclosure provides compounds and processes for the preparation of hypomethylating agents, including guadecitabine and salts thereof. The present disclosure also provides solid forms of guadecitabine sodium, including polymorphs that can exhibit decreased hygroscopicity and increased stability relative to other solid forms.

### CROSS REFERENCE

This application claims the benefit of U.S. Provisional Application No. 63/112,429, filed Nov. 11, 2020, which is hereby incorporated by reference in its entirety.

### BACKGROUND

DNA methylation is a post replicative chemical modification of DNA. Different cancers can be stratified by their abnormal DNA methylation profiles (degree of global or specific DNA methylation) and the hypermethylation of specific genes can be associated with the prognosis for gastric, lung, esophageal, pancreatic, and colon cancer. DNA methylation patterns can also be used to predict response or resistance to therapy in glioma and melanoma. Azacitidine and decitabine are two FDA approved hypomethylating agents (HMAs) that exert their therapeutic effect by inhibiting DNA methylation levels.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the present disclosure provides a compound of formula (IVa):

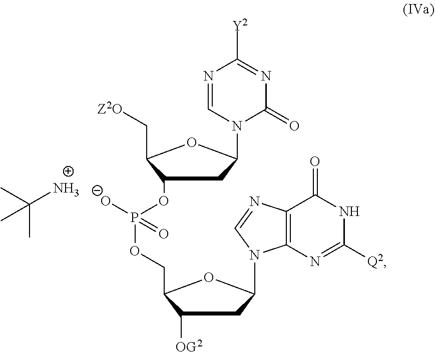

wherein: each Z² and G² is independently H or a protecting group; and each Y² and Q² is independently NH₂ or a protected primary amine.

In some embodiments, the present disclosure provides a process comprising contacting a solution with a base, wherein the solution comprises a compound of formula (III):

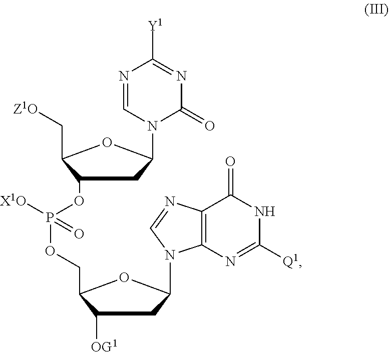

to provide an ion pair of formula (IVb):

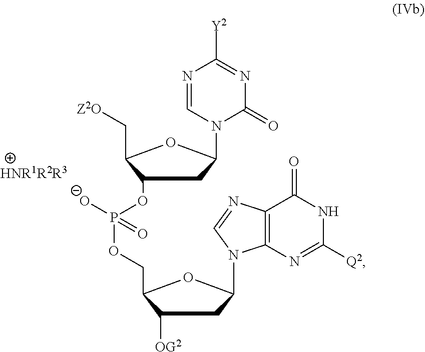

wherein:
the base is NR¹R²R³;
X¹ is 2-cyanoethyl, 2,2,2-trichloroethyl, 2,2,2-tribromoethyl, 2,2,2-trifluoroethyl, benzyl, p-chloroethyl, or p-nitroethyl;
R¹, R², and R³ are each independently H or branched or unbranched alkyl, wherein at least one of R¹, R², and R³ is not H; or R¹ is H or branched or unbranched alkyl and R² and R³ taken together with the atom to which R² and R³ are bound form a ring;
each Z¹, Z², G¹, and G² is independently H or a protecting group;
Y¹ is a protected primary amine; and
each Y², Q¹, and Q² is independently NH₂ or a protected primary amine.

In some embodiments, the present disclosure provides a process comprising: (i) contacting a solution with a base, wherein the solution comprises a first compound of formula (IV):

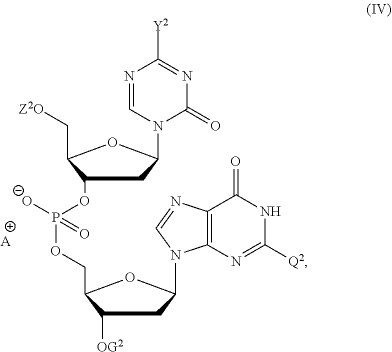

to provide a reaction mixture; and
(ii) contacting the reaction mixture with an acid to provide a second reaction mixture, wherein the second reaction mixture comprises a second compound, wherein the second compound is:

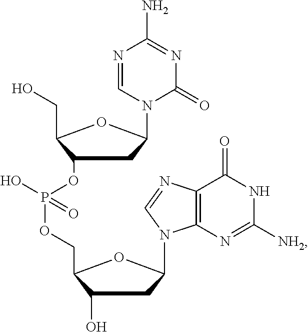

wherein:
A⁺ is an alkylammonium cation;
Z² and G² are each independently H, substituted or unsubstituted acetyl, or substituted or unsubstituted phenoxyacetyl; and
Y² and Q² are each independently NH₂, or a primary amine protected with substituted or unsubstituted acetyl, or substituted or unsubstituted phenoxyacetyl.

In some embodiments, the present disclosure provides a process comprising contacting a first solution with a lipase and an acetyl donor to provide a second solution, wherein the first solution comprises a compound of formula (Ia):

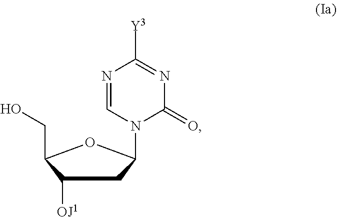

wherein the second solution comprises a compound of formula (Id):

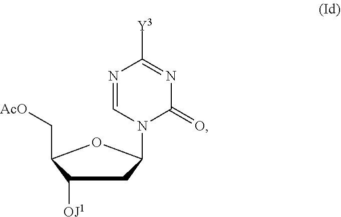

wherein:
the lipase is Novozym® 40086;
Y³ is NH₂ or a protected primary amine; and
J¹ is H, or a protecting group.

In some embodiments, the present disclosure provides a process for producing a polymorph of Compound 10:

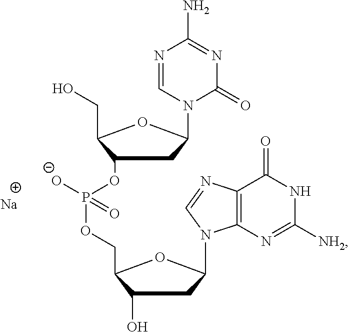 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

comprising:
(i) mixing Compound 10 with ethanol to provide a suspension;
(ii) filtering the suspension to provide a retentate; and
(iii) drying the retentate under reduced pressure to provide the polymorph, wherein the ethanol has a water content that is no more than 7% (w/w).

In some embodiments, the present disclosure provides a process for producing a polymorph of Compound 10, comprising drying under reduced pressure a solid form of Compound 10 to provide the polymorph, wherein the solid form of Compound 10 has an X-ray powder diffraction pattern that comprises peaks at 4.9°, 7.2°, and 10.0°±0.2 2θ as measured by X-ray powder diffraction using Cu K alpha radiation.

In some embodiments, the present disclosure provides a composition comprising a solid form of Compound 10, wherein the solid form exhibits an X-ray powder diffraction pattern substantially the same as the X-ray powder diffraction pattern shown in FIG. 6.

In some embodiments, the present disclosure provides a composition comprising a solid form of Compound 10, wherein the solid form has an X-ray powder diffraction pattern that comprises peaks at 10.1°, 11.2°, and 14°±0.2 2θ as measured by X-ray powder diffraction using Cu K alpha radiation.

In some embodiments, the present disclosure provides a composition comprising a solid form of Compound 10, wherein the solid form exhibits an X-ray powder diffraction pattern substantially the same as the X-ray powder diffraction pattern shown in FIG. 7.

In some embodiments, the present disclosure provides a composition comprising a solid form of Compound 10, wherein the solid form has an X-ray powder diffraction pattern that comprises peaks at 5.1°, 10.2°, and 11.2°±0.2 2θ as measured by X-ray powder diffraction using Cu K alpha radiation.

In some embodiments, the present disclosure provides a process for preparing Compound 10, the process comprising contacting a first mixture with a sodium cation source to provide a second mixture, wherein the first mixture comprises Compound 9:

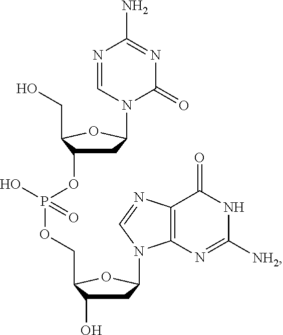

and dimethyl sulfoxide, and the second mixture comprises Compound 10.

In some embodiments, the present disclosure provides a process comprising:
(i) contacting a first mixture with ethanol to provide a second mixture, wherein the first mixture comprises Compound 10 and water;
(ii) cooling the second mixture to provide a precipitate; and
(iii) isolating the precipitate via filtration to provide a polymorph of Compound 10.

In some embodiments, the present disclosure provides a process comprising:
(i) contacting Compound 10 with a first mixture to provide a second mixture, wherein the first mixture comprises a solvent, wherein the solvent is a combination of water and ethanol;
(ii) heating the second mixture to a temperature of from about 30° C. to about 45° C.;
(iii) after the heating, cooling the second mixture to provide a precipitate; and
(iv) isolating the precipitate via filtration to provide a polymorph of Compound 10.

### BRIEF DESCRIPTION OF THE FIGURES

FIG. 1 is a chart summarizing the polymorphic form transitions observed when Compound 10 (guadecitabine sodium) is dried under various conditions.

FIG. 2 is a chart summarizing the effect of filter cake water content on Compound 10 polymorphic form output.

FIG. 3 is a chart summarizing the polymorphic form of Compound 10 observed as a function of water content of ethanol reslurry solvent.

FIG. 4 is a chart summarizing the effect of ethanol reslurry solvent temperature Compound 10 polymorphic form output.

FIG. 5 is a chart summarizing the polymorphic form of Compound 10 observed as a function of reslurry solvent composition comprising 7.1% to 28.6% (w/w) water in ethanol.

FIG. 6 depicts a diffractogram obtained from X-ray powder diffraction (XRPD) analysis of Form A of Compound 10 (guadecitabine sodium) using Cu K alpha radiation.

FIG. 7 depicts a diffractogram obtained from X-ray powder diffraction (XRPD) analysis of Form B of Compound 10 using Cu K alpha radiation.

FIG. 8 depicts a diffractogram obtained from X-ray powder diffraction (XRPD) analysis of Form C of Compound 10 using Cu K alpha radiation.

FIG. 9 depicts a diffractogram obtained from X-ray powder diffraction (XRPD) analysis of Form D of Compound 10 using Cu K alpha radiation.

FIG. 10 depicts a diffractogram obtained from X-ray powder diffraction (XRPD) analysis of Compound 10 as provided by the process detailed in EXAMPLE 4, Process B.

### DETAILED DESCRIPTION

This application relates to processes for manufacture of dinucleotides derived from decitabine including guadecitabine and salts thereof, and synthetic intermediates useful for said processes. The present disclosure also provides solid forms of guadecitabine sodium, including polymorphs that can exhibit decreased hygroscopicity and increased stability relative to other solid forms.

In some embodiments is provided solid forms of guadecitabine sodium, namely, forms A, B, C, and D. Forms A and B can be less hygroscopic relative to other crystalline forms of guadecitabine sodium, and can therefore be less susceptible to degradative processes promoted by water. For example, impurities can be formed by, for example, opening of the triazine ring of guadecitabine with water, or opening of the triazine ring with water followed by basic cleavage of the intermediate formamide.

### Compounds of the Disclosure.

Provided herein are compounds that can be useful in the manufacture of dinucleotide compounds. In some embodiments, provided herein is a compound of formula (I):

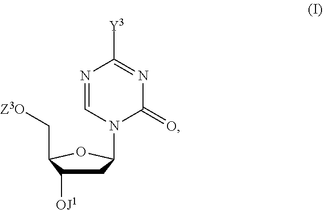

wherein:
Z³ is H or a protecting group;
Y³ is NH₂ or a protected primary amine; and
J¹ is H, a protecting group, or —P(OX²)V¹, wherein
X² is 2-cyanoethyl, 2,2,2-trichloroethyl, 2,2,2-tribromoethyl, 2,2,2-trifluoroethyl, benzyl, p-chloroethyl, or p-nitroethyl; and
V¹ is N(R⁴)₂, wherein each R⁴ is C₁₋₆alkyl or aryl.

In some embodiments, Z³ and J¹ are each independently H, substituted or unsubstituted acetyl, substituted or unsubstituted phenoxyacetyl, substituted or unsubstituted ethoxymethyl, substituted or unsubstituted benzoyl, or a silyl protecting group. In some embodiments, Z³ and J¹ are each independently H, substituted or unsubstituted acetyl, or substituted or unsubstituted phenoxyacetyl. In some embodiments, Z³ is H, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, Z³ and J¹ are each independently H, acetyl, Pac, Tac, or iPr-Pac. In some embodiments, Z³ and J¹ are each independently H or acetyl. In some embodiments, Z³ is substituted or unsubstituted acetyl. In some embodiments, Z³ is acetyl. In some embodiments, J¹ is H. In some embodiments, Z³ is acetyl and J¹ is H. In some embodiments, Z³ is H and J¹ is H.

In some embodiments, Y³ is NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or an amidine group. In some embodiments, Y³ is NH₂, or a primary amine protected with substituted or unsubstituted acetyl, substituted or unsubstituted phenoxyacetyl, or an amidine group. In some embodiments, Y³ is NH₂ or a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, Y³ is NH₂, or a primary amine protected with acetyl, Pac, Tac, iPr-Pac, or ═CHN(CH₃)₂. In some embodiments, Y³ is NH₂ or N═CHN(CH₃)₂.

In some embodiments, J¹ is —P(OX²)V¹, wherein X² is 2-cyanoethyl, 2,2,2-trichloroethyl, 2,2,2-tribromoethyl, 2,2,2-trifluoroethyl, benzyl, p-chloroethyl, or p-nitroethyl; and V¹ is N(R⁴)₂, wherein each R⁴ is C₁₋₆alkyl. In some embodiments, X² is 2-cyanoethyl, and each R⁴ is isopropyl. In some embodiments, J¹ is H.

In some embodiments, the compound is of formula (Ia):

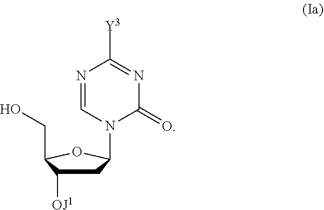

In some embodiments, the compound is of formula (Ib):

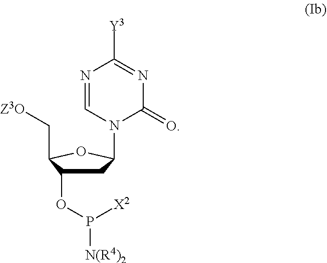

In some embodiments, the compound is of formula (Ic):

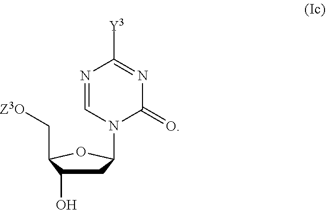

In some embodiments, the compound is of formula (Id):

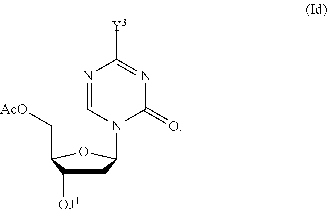

In some embodiments, the compound is Compound 1, which has the structure:

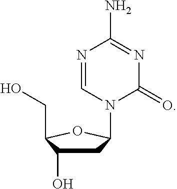

In some embodiments, the compound is Compound 2, which has the structure:

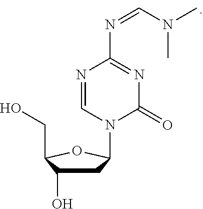

In some embodiments, the compound is Compound 3, which has the structure:

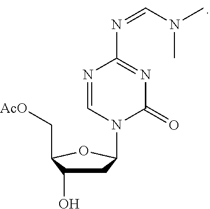

In some embodiments, the compound is Compound 4, which has the structure:

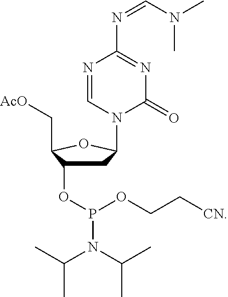

In some embodiments, provided herein is a compound of formula (II):

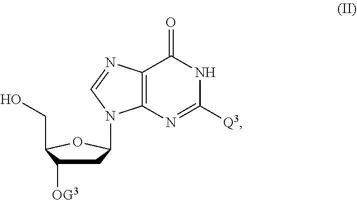

wherein:
G³ is H or a protecting group; and
Q³ is independently NH₂ or a protected primary amine.

In some embodiments, Q³ is NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or an amidine group. In some embodiments, Q³ is NH₂, or a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, Q³ is NH₂, or a primary amine protected with ═CHN(CH₃)₂, acetyl, Pac, Tac, iPr-Pac. In some embodiments, Q¹ is NH₂ or NH(Tac). In some embodiments, Q¹ is NH(Tac).

In some embodiments, G³ is H, substituted or unsubstituted acetyl, substituted or unsubstituted phenoxyacetyl, substituted or unsubstituted ethoxymethyl, substituted or unsubstituted benzoyl, or a silyl protecting group. In some embodiments, G³ is H, substituted or unsubstituted acetyl, or substituted or unsubstituted phenoxyacetyl. In some embodiments, G³ is H, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, G³ is H, acetyl, Pac, Tac, or iPr-Pac. In some embodiments, G³ is Tac.

In some embodiments, the compound is Compound 5, which has the structure:

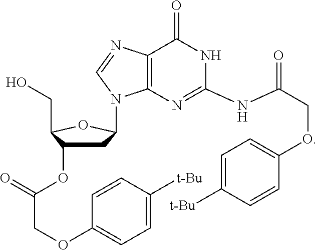

In some embodiments, provided herein is a compound of formula (III):

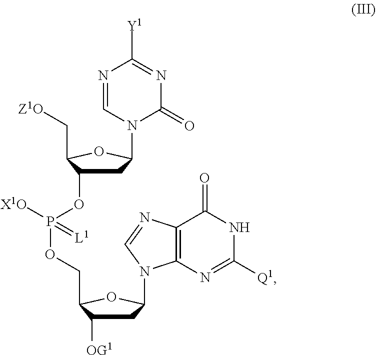

wherein:
X¹ is H, 2-cyanoethyl, 2,2,2-trichloroethyl, 2,2,2-tribromoethyl, 2,2,2-trifluoroethyl, benzyl, p-chloroethyl, or p-nitroethyl;
L¹ is absent, or O or S.
each Z¹ and G¹ is independently H or a protecting group; and
each Y¹ and Q¹ is independently NH₂ or a protected primary amine.

In some embodiments, L¹ is 0 or absent. In some embodiments, L¹ is 0.

In some embodiments, the compound is of formula (Ma):

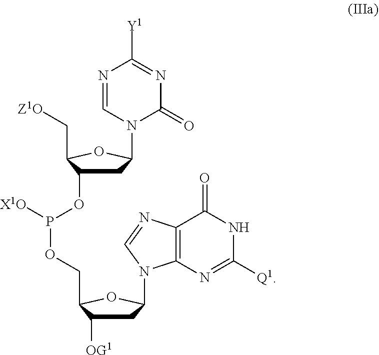

In some embodiments, the compound is of formula (Mb):

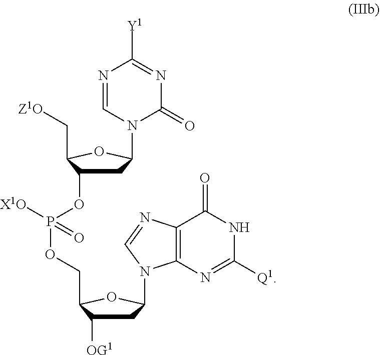

In some embodiments, each Z¹ and G¹ is independently H, substituted or unsubstituted acetyl, substituted or unsubstituted phenoxyacetyl, substituted or unsubstituted ethoxymethyl, substituted or unsubstituted benzoyl, or a silyl protecting group. In some embodiments, each Z¹ and G¹ are each independently H, substituted or unsubstituted acetyl, or substituted or unsubstituted phenoxyacetyl. In some embodiments, each Z¹ and G¹ is independently H, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, each Z¹ and G¹ is independently H, acetyl, Pac, Tac, or iPr-Pac.

In some embodiments, each Y¹ and Q¹ is independently NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or an amidine group. In some embodiments, each Y¹ and Q¹ are each independently NH₂, or a primary amine protected with substituted or unsubstituted acetyl, or substituted or unsubstituted phenoxyacetyl. In some embodiments, each Y¹ and Q¹ is independently NH₂, or a primary amine protected with benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, each Y¹ and Q¹ is independently NH₂, or a primary amine protected with acetyl, Pac, Tac, iPr-Pac.

In some embodiments, Z¹ is H or acetyl. In some embodiments, G¹ is H or Tac. In some embodiments, Y¹ is NH₂ or NH(Tac). In some embodiments, G¹ is NH₂ or NH(Tac). In some embodiments, Y¹ and Q¹ are each independently selected from the group consisting of NH₂ and NH(Tac).

In some embodiments, Y¹ is a protected primary amine. In some embodiments, Y¹ is a protected primary amine, and Q¹ is independently NH₂ or a protected primary amine. In some embodiments, Y¹ is a protected primary amine, Q¹ is independently NH₂ or a protected primary amine, and each Z¹ and G¹ is independently H or a protecting group.

In some embodiments, Y¹ is a primary amine protected with substituted or unsubstituted acetyl, substituted or unsubstituted phenoxyacetyl, substituted or unsubstituted ethoxymethyl, substituted or unsubstituted benzoyl, a silyl protecting group, or an amidine group. In some embodiments, Y¹ is a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, Y¹ is a primary amine protected with ═C(CH₃)₂, acetyl, Pac, Tac, iPr-Pac. In some embodiments, Y¹ is N═CHN(CH₃)₂ or NH(Tac).

In some embodiments, Q¹ is NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or an amidine group. In some embodiments, Q¹ is NH₂, or a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, Q¹ is NH₂, or a primary amine protected with ═CHN(CH₃)₂, acetyl, Pac, Tac, iPr-Pac. In some embodiments, Q¹ is NH₂ or NH(Tac).

In some embodiments, the compound is Compound 6, which has the structure:

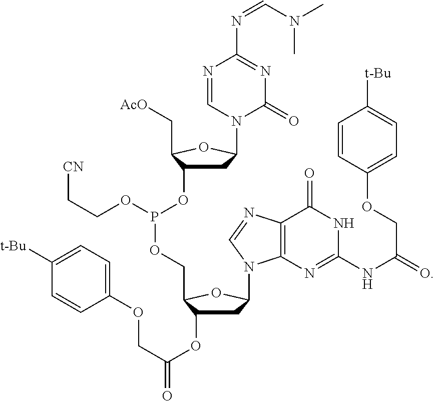

In some embodiments, the compound is Compound 7, which has the structure:

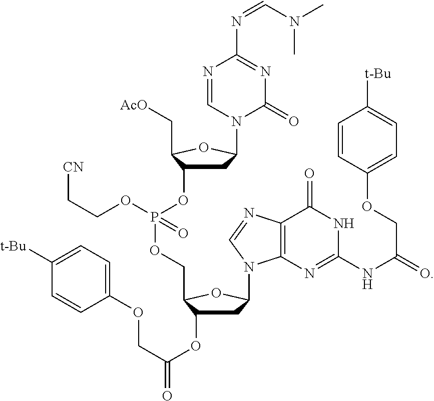

In some embodiments, the compound is Compound 9, which has the structure:

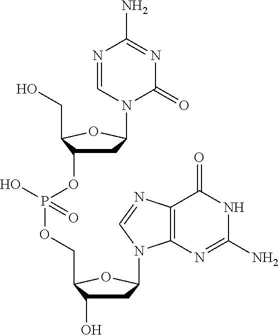

In some embodiments, provided herein is a compound of formula (IV):

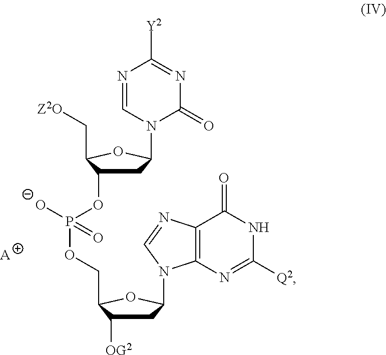

wherein:
each Z² and G² is independently H or a protecting group;
each Y² and Q² is independently NH₂ or a protected primary amine; and
A⁺ is an alkali metal cation, or an organic cation comprising a nitrogen atom.

In some embodiments, A⁺ is an organic cation comprising a nitrogen atom. In some embodiments, A⁺ is an alkali metal cation. In some embodiments, A⁺ is Na⁺. In some embodiments, A⁺ is an alkylammonium cation. In some embodiments, A⁺ is ⁺HNR¹R²R³, wherein R¹, R², and R³ are each independently H or branched or unbranched alkyl, wherein at least one of R¹, R², and R³ is not H; or R¹ is H or branched or unbranched alkyl and R² and R³ are taken together with the atom to which they are bound to form a ring.

In some embodiments, the compound is of formula (IVa):

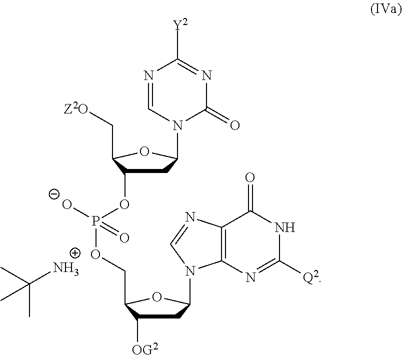

In some embodiments, the compound is of formula (IVb):

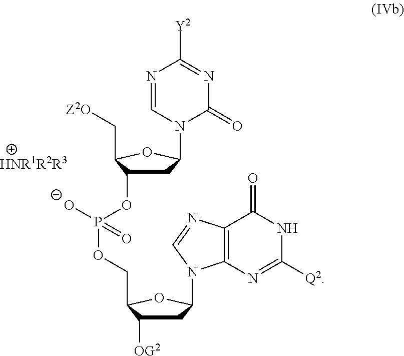

In some embodiments, each Z² and G² is independently H, substituted or unsubstituted acetyl, substituted or unsubstituted phenoxyacetyl, substituted or unsubstituted ethoxymethyl, substituted or unsubstituted benzoyl, or a silyl protecting group. In some embodiments, each Z² and G² are each independently H, substituted or unsubstituted acetyl, or substituted or unsubstituted phenoxyacetyl. In some embodiments, each Z² and G² is independently H, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, each Z² and G² is independently H, acetyl, Pac, Tac, or iPr-Pac.

In some embodiments, each Y² and Q² is independently NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or an amidine group. In some embodiments, each Y² and Q² are each independently NH₂, or a primary amine protected with substituted or unsubstituted acetyl, or substituted or unsubstituted phenoxyacetyl. In some embodiments, each Y² and Q² is independently NH₂, or a primary amine protected with benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, each Y² and Q² is independently NH₂, or a primary amine protected with acetyl, Pac, Tac, iPr-Pac.

In some embodiments, Z² is H or acetyl. In some embodiments, G² is H or Tac. In some embodiments, Y² is NH₂ or NH(Tac). In some embodiments, G² is NH₂ or NH(Tac). In some embodiments, Y² and Q² are each independently selected from the group consisting of NH₂ and NH(Tac).

In some embodiments, R¹ and R² are each H, and R³ is tert-butyl. In some embodiments, R¹ and R² are each H, and R³ is sec-butyl. In some embodiments, R¹ and R² are each H, and R³ is n-butyl. In some embodiments, R¹, R², and R³ are each methyl, ethyl, n-propyl, or n-butyl. In some embodiments, R¹ and R² are each isopropyl, and R³ is H. In some embodiments, R¹ and R² are each isopropyl, and R³ is ethyl. In some embodiments, R¹ is H, and R² and R³ are taken together with the atom to which they are bound to form 5-membered or 6-membered saturated ring.

In some embodiments, the compound is Compound 8, which has the structure:

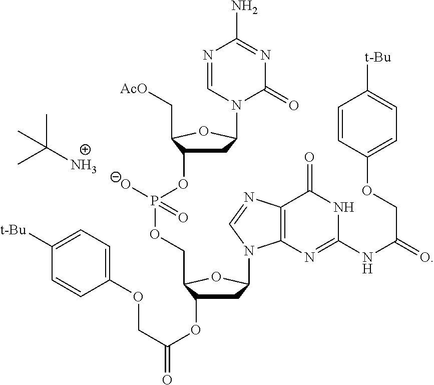

Compounds of formula (IVb), such as Compound 8, can be useful in processes for the manufacture of dinucleotide therapeutics that use precipitation and filtration as a replacement or complement process to chromatographic purification. In some embodiments, a compound of formula (Mb) (e.g. Compound 7) can be contacted with a compound of the formula NR¹R²R³ (e.g. tert-butylamine) to afford a compound of formula IVb, where R¹, R², and R³ are as defined above. In such cases, the compound of the formula NR¹R²R³ can serve as both a nucleophilic reagent for the removal of amidine and phospholinker protecting groups in a compound of formula (IIIb) (e.g. where X¹ is 2-cyanoethyl and Y¹ or Q¹ is N═CHN(Me)₂), and a reagent for the formation of a salt (e.g. Compound 8) that can be purified by precipitation and filtration.

In some embodiments, the compound is Compound 10, which has the structure:

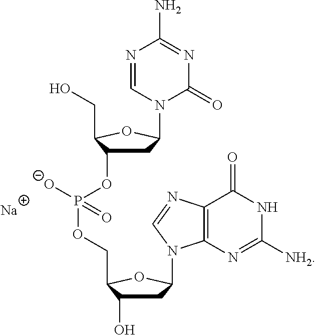

In some embodiments, the compound is Compound 11, which has the structure:

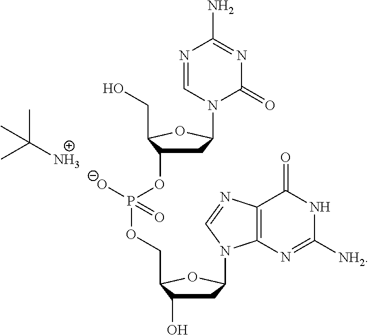

In some embodiments, the compound is Compound 12, which has the structure:

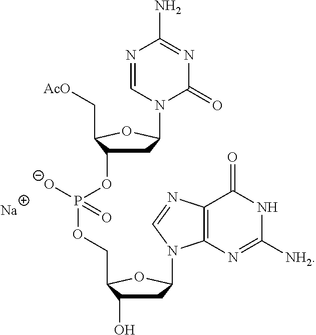

### Protecting Groups.

A compound disclosed herein can be functionalized with a protecting group, whereby a reactive group is chemically transformed into a protected group that does not react under conditions where the non-protected group reacts. For example, a primary amine group that is protected with a protecting group can have the structure —NHPg, where Pg is a protecting group. In another example, a primary amine that is protected with a formamidine group can have the structure —N═CHN(CH₃)₂.

Suitable protecting groups include, but are not limited to acyl groups (e.g. acetyl (Ac), benzoyl (Bz), isobutyryl (Ib), methoxyacetyl, isopropoxyacetyl, levulinyl, 4-pentenoyl, 4-nitrophenylethyloxycarbonyl, phenacetyl, 9-fluorenylmethoxycarbonyl, α-phenylcinnamoyl, phenoxyacetyl (Pac), substituted phenoxyacetyls including 2-chlorophenoxyacetyl, 4-(tert-butyl)phenoxy acetyl (Tac), and (4-isopropylphenoxy)acetyl (iPr-Pac), diphenylacetyl, 3-methoxy-4-phenoxybenzoyl, 4-methylbenzoyl, 4-methoxybenzoyl, 3,4-dichlorobenzoyl, 1,8-naphthaloyl, and allyloxycarbonyl), silyl groups (e.g. trimethylsilyl, tert-butyldimethylsilyl, triisopropylsilyl, tribenzylsilyl, triphenylsilyl, ethers and silyl ethers (e.g. [(triisopropylsilyl)oxy]methyl, [(2-nitrobenzyl)oxy]methyl, 2-(4-tolyl sulfonyl)ethoxy methyl, 1-(2-cyanoethoxy)ethyl, 2′-cyanoethoxymethyl), and unsubstituted or substituted methyl and ethyl (e.g. 2-cyanoethyl, 2,2,2-trichloroethyl, 2,2,2-tribromoethyl, 2,2,2-trifluoroethyl, benzyl, p-chloroethyl, or p-nitroethyl). Various examples of protecting group components are described in P. G. M. Wuts, Greene's Protective Groups in Organic Synthesis, 5th Edition, John Wiley & Sons (2014).

In some embodiments, intermediates comprising protected primary amines are required. Suitable protecting groups for primary amines include, for example, those recited above, as well as amidine groups (e.g. (dimethylamino)methylene (formamidine), (di-n-butylamino)methylene, (diisopropylamino)methylene, 1-(dimethylamino)ethylidene, (diisobuylamino)methylene, and N-methylpyrrolidin-2-ylidene).

### Optional Substituents.

The compounds of the present disclosure can be substituted or unsubstituted. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, and ester groups.

Non-limiting examples of alkyl and alkylene groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, triazine, pyrimidine, pyrazine, pyridazine, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

### Compositions.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Synthetic Transformations.

The synthetic transformations of the present disclosure can be proportionally scaled to provide a larger quantity of product. The total mass of any one compound (e.g. Compounds 1-10) used as a reactant in a single batch reaction disclosed herein can be least about 1 kg, at least about 2 kg, at least about 3 kg, at least about 4 kg, at least about 5 kg, at least about 6 kg, at least about 7 kg, at least about 8 kg, at least about 9 kg, at least about 10 kg, at least about 11 kg, at least about 12 kg, at least about 13 kg, at least about 14 kg, at least about 15 kg, at least about 16 kg, at least about 17 kg, at least about 18 kg, at least about 19 kg, at least about 20 kg, at least about 22 kg, at least about 24 kg, at least about 26 kg, at least about 28 kg, or at least about 30 kg.

The synthetic transformations of the present disclosure can be carried in the presence of a solvent. Nonlimiting examples of solvents include acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol dimethyl ether), 1,2-dimethoxyethane (glyme, DME), dimethylacetamide (DMA), dimethylformamide (DMF), dimethyl sulfoxide (DMSO), 1,4-dioxane, ethanol, ethyl acetate, ethylene glycol, glycerin, heptane, hexamethylphosphoramide (HMPA), Hexamethylphosphorous triamide (HMPT), hexane, methanol, methyl tert-butyl ether (MTBE), methylene chloride, N-methyl-2-pyrrolidinone (NMP), nitromethane, pentane, petroleum ether (ligroine), 1-propanol, 2-propanol (isopropanol, IPA), pyridine, tetrahydrofuran (THF), 2-methyltetrahydrofuran (MeTHF), toluene, triethyl amine, water, o-xylene, m-xylene, and p-xylene.

In some embodiments, guadecitabine sodium is prepared according to the following scheme:

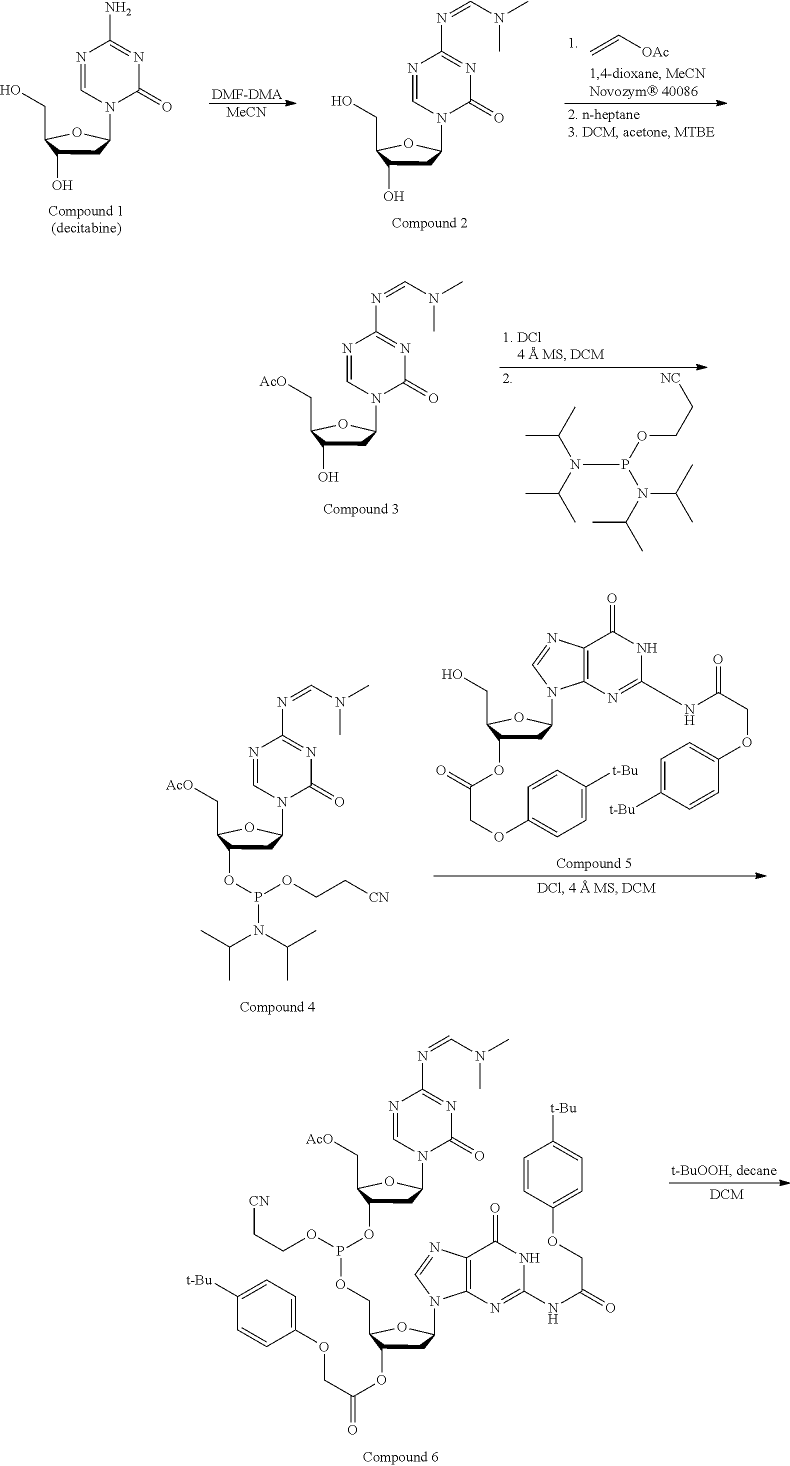

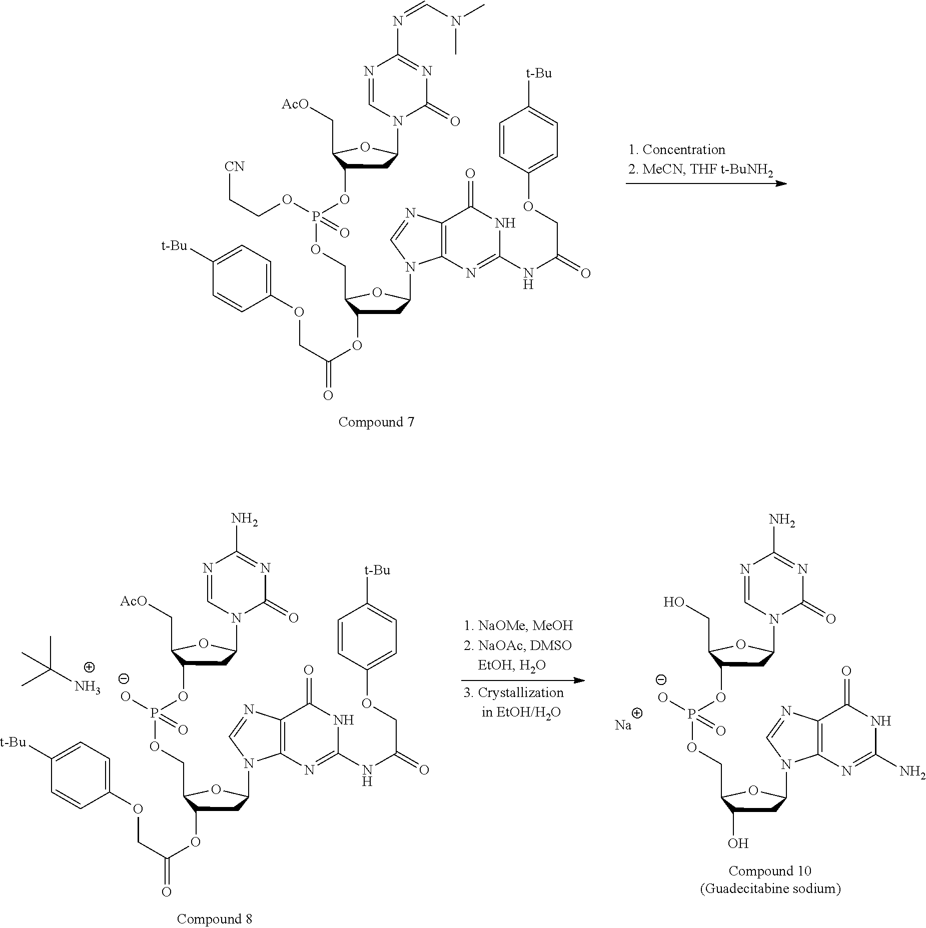

### Protection of Decitabine.

In one embodiment, provided herein is a process comprising contacting decitabine (Compound 1) with a formamide, (e.g. N,N-dimethylforamide or N,N-dibutylformamide), or an acetal thereof (e.g. N,N-dimethylformamide dimethylacetal), to provide a formamidine. In some embodiments, the formamide acetal is N,N-dimethylformamide dimethylacetal (DMF-DMA) and the formamidine is Compound 2. For example, decitabine can be first combined with an organic solvent to provide a solution. In some embodiments, the concentration of decitabine in the solution is from about 5% to about 20% (w/w).

Non-limiting examples of suitable organic solvents include acetonitrile, tetrahydrofuran, 2-methyltetrahydrofuran, dichloromethane, 1,2-dichloroethane, toluene, 1,4-dioxane, mixtures of dioxane isomers, dimethylformamide, dimethylacetamide, N-methyl pyrrolidone, methyl tert-butyl ether, cyclopentyl methyl ether, and mixtures thereof. In some embodiments, the organic solvent is acetonitrile. In some embodiments, the concentration of decitabine in the solution is from about 8% to about 12% (w/w), such as about 8%, about 9%, about 10%, about 11%, or about 12% (w/w).

Upon combination of the decitabine with the solvent, the solution can be stirred. The reaction can be stirred for a minimum period of time, such as for at least 10 minutes, at least 12 minutes, at least 13 minutes, at least 14 minutes, or at least 15 minutes. After stirring for the specified duration, the solution can then be combined with the formamide acetal, such as DMF-DMA. In some embodiments, the solution is contacted with from about 2.5 to about 3.1 molar equivalents of DMF-DMA relative to the starting molar quantity of decitabine. After addition of the formamide acetal, the reaction is further stirred. The stirring can be conducted at room temperature, or at a temperature from about 10° C. to about 40° C., from about 15° C. to about 35° C., or from about 18° C. to about 30° C.

In some embodiments, the reaction is stirred until the reaction attains a completion criteria based on the consumption of decitabine (i.e. conversion), such as at least about 90%, at least about 95%, at least about 97% at least about 98%, at least about 99%, at least about 99.5%, at least about 99.7%, or at least about 99.9% of the starting quantity of decitabine is consumed as determined by assay (e.g. HPLC, NMR, IR, Raman).

After the completion criteria is met, the product (e.g. Compound 2) can be isolated by filtration or centrifugation to provide a retentate. The retentate is optionally washed or triturated with an organic solvent in which the product is not completely soluble (e.g. acetonitrile) and then dried.

### Acyl Transfer.

In some embodiments, the disclosure provides a process comprising contacting a first solution with a lipase and an acyl donor to provide a second solution, wherein the first solution comprises a solvent and a nucleoside, and the second solution comprises a 5′-acylated nucleoside product.

In some embodiments, the nucleoside is of formula (Ia):

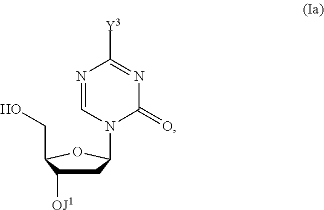

and the 5′-acylated nucleoside product is of formula (Id),

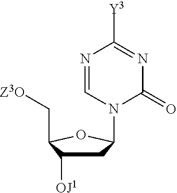

wherein:
Z³ is H or a protecting group;
Y³ is NH₂ or a protected primary amine; and
J¹ is H, a protecting group, or —P(OX²)V¹, wherein
X² is 2-cyanoethyl, 2,2,2-trichloroethyl, 2,2,2-tribromoethyl, 2,2,2-trifluoroethyl, benzyl, p-chloroethyl, or p-nitroethyl; and V¹ is N(R⁴)₂, wherein each R⁴ is C₁₋₆alkyl or aryl.

In some embodiments, Z³ and J¹ are each independently H, substituted or unsubstituted acetyl, substituted or unsubstituted phenoxyacetyl, substituted or unsubstituted ethoxymethyl, substituted or unsubstituted benzoyl, or a silyl protecting group. In some embodiments, Z³ and J¹ are each independently H, substituted or unsubstituted acetyl, or substituted or unsubstituted phenoxyacetyl. In some embodiments, Z³ is H, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, Z³ and J¹ are each independently H, acetyl, Pac, Tac, or iPr-Pac. In some embodiments, Z³ and J¹ are each independently H or acetyl. In some embodiments, Z³ is substituted or unsubstituted acetyl. In some embodiments, Z³ is acetyl. In some embodiments, J¹ is H. In some embodiments, Z³ is acetyl and J¹ is H. In some embodiments, Z³ is H and J¹ is H.

In some embodiments, the nucleoside is Compound 1 or Compound 2. In some embodiments, the nucleoside is Compound 2 and the 5′-acylated nucleoside product is Compound 3.

In some embodiments, the acyl donor comprises an ester moiety or an anhydride moiety. In some embodiments, the acyl donor has a molecular weight that is no more than 300. In some embodiments, the acyl donor is an acetyl donor, which can include, for example, anhydrides, mixed anhydrides comprising an acetyl moiety, C₁-C₁₂ alkyl acetates, or C₁-C₁₂ alkenyl acetates. In some embodiments, the acyl donor is an acetyl donor, and Z³ is acetyl. In some embodiments, the acetyl donor is vinyl acetate, isopropenyl acetate, ethyl acetate, or acetic anhydride.

In some embodiments, the lipase is a 1,3-specific lipase. In some embodiments, the lipase is immobilized on an acrylic resin. In some embodiments, the lipase is immobilized on a silica gel carrier. In some embodiments, the lipase is a lipase isolated from Rhizomucor miehei or Mucor miehei. In some embodiments, the lipase is Novozym® 40086. In some embodiments, the lipase is another biological material that has substantially the same biological or industrial activity as Novozym® 40086.

The solvent can be an organic solvent, which can be, for example, 1,4-dioxane, or a mixture of solvents, such as 1,4-dioxane and acetonitrile.

The nucleoside can be first combined with 1,4-dioxane to provide a first solution, wherein the concentration of the nucleoside in the mixture is from about 0.01% to about 0.1%, from about 0.01% to about 0.08%, from about 0.01% to about 0.06%, from about 0.01% to about 0.05%, or from about 0.01% to about 0.04%.

After formation of the first solution, the lipase and the acyl donor can be added to the first solution to provide a second solution. The amount of acyl donor can be at least about 1 molar equivalent of acyl donor relative to the starting molar quantity of the compound of nucleoside, such as at least about 1 molar equivalent, at least about 2 molar equivalents, at least about 5 molar equivalents, at least about 8 molar equivalents, at least about 9 molar equivalents, from about 1 to about 40 molar equivalents, from about 1 to about 30 molar equivalents, from about 1 to about 20 molar equivalents, from about 1 to about 15 molar equivalents, from about 5 to about 20 molar equivalents, or from about 5 to about 15 molar equivalents of acyl donor. In some embodiments, the acyl donor is vinyl acetate.

A polar organic solvent can be used to rinse the acyl donor (e.g., vinyl acetate) into the first solution, such as, for example, acetonitrile. In some embodiments, the amount of acetonitrile added to the mixture is from about 0.1 kg to about 0.5 kg per every 1 kg of 1,4-dioxane in the mixture.

After the addition of the acyl donor, the second solution can be stirred. The stirring can be conducted at room temperature, or at a temperature from about 10° C. to about 50° C., from about 20° C. to about 45° C., from about 25° C. to about 45° C., or from about 25° C. to about 40° C.

The second solution can be stirred until the reaction attains a completion criteria based on the consumption of the compound of formula (Ia) (i.e. conversion), such as at least about 90%, at least about 95%, at least about 97% at least about 98%, at least about 99%, at least about 99.5%, at least about 99.7%, or at least about 99.9% of a starting quantity of the nucleoside is consumed as determined by assay (e.g., HPLC, NMR, IR, or Raman).

After the completion criteria is met, the second solution can be filtered to provide a filtrate. The filter cake can be washed with a solvent or solvent mixture, such as 1,4-dioxane and acetonitrile. An antisolvent relative to the acylated product can be added to the filtrate to provide a suspension. In some embodiments, the antisolvent is n-heptane. In some embodiments, the amount of n-heptane added to the filtrate is from about 80 kg to about 120 kg per every 1 kg of the starting quantity of the nucleoside, or from about 90 kg to about 100 kg per every 1 kg of the starting quantity of the nucleoside. The n-heptane can be added to the filtrate at a controlled rate, such as a rate of no more than 100 kg/min. After addition of the antisolvent, the resulting mixture can be stirred for a period of time, such as for at least 30 minutes. The suspension can be filtered to provide a retentate, and the retentate can be washed with the antisolvent to provide a solid comprising the acylated product, which can be optionally dried until a free-flowing powder is obtained.

### Purification of Compounds of Formula (I).

A compound of formula (I) can be purified by recrystallization. In one embodiment, a compound of formula (I), such as Compound 3, is combined with a an organic solvent in which the compound of formula (I) is soluble, such as, for example, dichloromethane, to provide a third solution. In some embodiments, the amount of dichloromethane that is combined with the compound of formula (I) is from about 14 to about 25 kilograms dichloromethane per 1 kg of the compound of formula (I).

The third solution can then be stirred until a clear solution is observed at room temperature, or at a temperature of no more than about 25° C. Acetone can be contacted with the clear solution. In some embodiments, the amount of acetone that is contacted is at least 0.4 kg for every 1 kg of dichloromethane in the clear solution, such as from about 0.4 to about 0.6 kg of acetone for every 1 kg of dichloromethane in the clear solution. One or more seed crystals of the compound of formula (I) can then be added, and the resulting mixture can be stirred for a period of time, such as for at least about 10 minutes.

After stirring, a portion of the solvent can be removed by distillation under reduced pressure. A solvent can be added to the concentrate to provide a mixture that crystalizes the product from the solution phase. In some embodiments, the product is crystallized from the solvent, wherein the solvent comprises acetone, tetrahydrofuran, 2-methyltetrahydrofuran, acetonitrile, dichloromethane, 1,2-dichloroethane, toluene, 1,4-dioxane, mixtures of dioxane isomers, dimethylformamide, dimethylacetamide, N-methyl pyrrolidone, methyl tert-butyl ether (MTBE), cyclopentyl methyl ether, isobutanol, isopropanol, ethanol, methanol, ethyl acetate, isopropyl acetate, isobutyl acetate, n-heptane or isomers thereof, n-hexane or an isomer thereof, n-pentane or an isomer thereof, or mixtures thereof. In some embodiments, the solvent comprises acetone and MTBE. In some embodiments, the solvent comprises about a 1:2 ratio (w/w) to acetone to MTBE.

The mixture can be stirred for a period of time such that a suspension is observed, such as at least about 60 minutes to about 48 hours. The mixture can be maintained at a certain temperature or range of temperatures, such as a temperature between 10° C. and 30° C. After stirring, the mixture can be filtered to provide a retentate, which can be optionally washed with a solvent, such as an aliphatic or ethereal solvent, and then dried. In some embodiments, the retentate is washed with MTBE and n-heptane.

### Installation of Phospholinker.

In some embodiments, the disclosure provides a process comprising contacting a compound of formula (Ic):

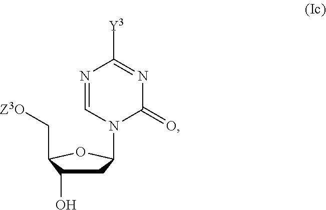

with a phosphorodiamidite in the presence of a solvent to provide a reaction mixture comprising a phosphoramidite of formula (Ib):

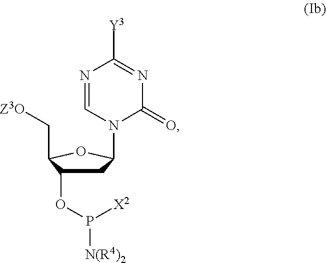

wherein
Z³ is H or a protecting group;
Y³ is NH₂ or a protected primary amine;
X² is 2-cyanoethyl, 2,2,2-trichloroethyl, 2,2,2-tribromoethyl, 2,2,2-trifluoroethyl, benzyl, p-chloroethyl, or p-nitroethyl; and
V¹ is N(R⁴)₂, wherein each R⁴ is independently C₁₋₆ alkyl or aryl.

In some embodiments, the phosphorodiamidite is of the formula (V²)₂P(OX³), wherein X³ is 2-cyanoethyl, 2,2,2-trichloroethyl, 2,2,2-tribromoethyl, 2,2,2-trifluoroethyl, benzyl, p-chloroethyl, or p-nitroethyl; and V² is N(R⁵)₂, wherein each R⁵ is C₁₋₆alkyl or aryl. In some embodiments, the phosphorodiamidite is a 3-((bis(C₁₋₆alkyl)phosphanyl)oxy)propanenitrile. In some embodiments, the phosphorodiamidite is 3-((bis(diisopropylamino)phosphanyl)oxy)propanenitrile.

Suitable solvents include halogenated solvents. In some embodiments, the solvent comprises dichloromethane. In some embodiments, the solvent contains no more than about 0.5% w/w water, no more than about 0.1% w/w water, no more than about 0.05% w/w water, or no more than about 0.01% w/w water.

In some embodiments, the contacting further comprises contacting the phosphorodiamidite in the presence of a coupling activator. Suitable coupling activators include, but are not limited to 1H-tetrazole and its derivatives such as N-nitrophenyl-1H-tetrazole, 5-(bis-3,5-trifluoromethylphenyl-1H-tetrazole, 5-ethylthio-1H-tetrazole, 5-benzylthio-1H-tetrazole, and 5-[3,5-bis(trifluoromethyl)phenyl)]-1H-tetrazole, imidazole activators such as 4,5-dicyanoimidazole, and 1-hydroxy-benzotriazole and 3-nitrotriazole activators.

In some embodiments, the process further comprises contacting the reaction mixture comprising the phosphoramidite with a compound of formula (II):

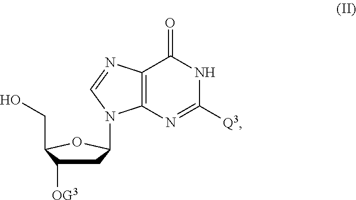

wherein:
to provide a second reaction mixture comprising a phosphite, wherein
G³ is H or a protecting group; and
Q³ is independently NH₂ or a protected primary amine.

In some embodiments, Q³ is NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or an amidine group. In some embodiments, Q³ is NH₂, or a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, Q³ is NH₂, or a primary amine protected with ═CHN(CH₃)₂, acetyl, Pac, Tac, iPr-Pac. In some embodiments, Q³ is NH₂ or NH(Tac). In some embodiments, Q³ is NH(Tac).

In some embodiments, G³ is H, substituted or unsubstituted acetyl, substituted or unsubstituted phenoxyacetyl, substituted or unsubstituted ethoxymethyl, substituted or unsubstituted benzoyl, or a silyl protecting group. In some embodiments, G³ is H, substituted or unsubstituted acetyl, or substituted or unsubstituted phenoxyacetyl. In some embodiments, G³ is H, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, G³ is H, acetyl, Pac, Tac, or iPr-Pac. In some embodiments, G³ is Tac.

In some embodiments, the phosphite is a compound the formula (Ma):

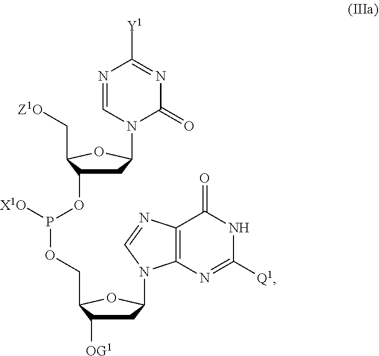

wherein:
X¹ is H, 2-cyanoethyl, 2,2,2-trichloroethyl, 2,2,2-tribromoethyl, 2,2,2-trifluoroethyl, benzyl, p-chloroethyl, or p-nitroethyl;
each Z¹ and G¹ is independently H or a protecting group; and
each Y¹ and Q¹ is independently NH₂ or a protected primary amine.

In some embodiments, the process further comprises contacting the reaction mixture comprising the phosphoramidite with a solution of the compound of formula (II) in a second solvent to provide the phosphite of formula (Ma). In some embodiments, the phosphoramidite is Compound 4, and the phosphite is Compound 6. In some embodiments, the second solvent is a halogenated solvent, such as dichloromethane or 1,2-dichloroethane. In some embodiments, the second solvent contains no more than about 0.5% w/w water, no more than about 0.1% w/w water, no more than about 0.05% w/w water, or no more than about 0.01% w/w water.

In some embodiments, the process further comprises contacting the second reaction mixture with an oxidant to provide a third reaction mixture comprising a phosphate of formula (IIIb):

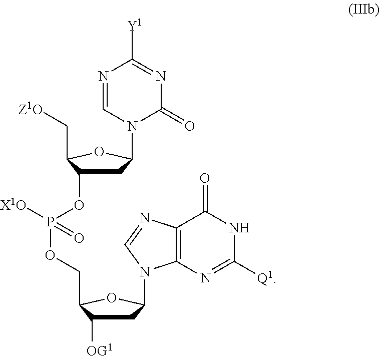

In some embodiments, the oxidant comprises a peroxide. In some embodiments, the oxidant is a perbenzoic acid. In some embodiments, the oxidant is tert-butyl hydroperoxide.

In some embodiments, the process further comprises contacting the third reaction mixture with tert-butylamine to provide an ion pair of formula (IVa):

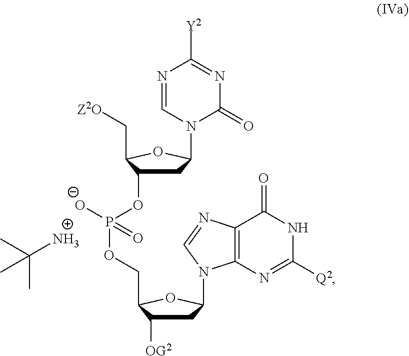

wherein:
each Z² and G² is independently H or a protecting group; and
each Y² and Q² is independently NH₂ or a protected primary amine.

In some embodiments, Y¹ is N═CHN(CH₃)₂ and Y² is NH₂. In some embodiments, the phosphite is Compound 6, the phosphate is Compound 7, and the ion pair is Compound 8.

In some embodiments, the solvent is dichloromethane. In some embodiments, the process further comprises replacing a quantity of the dichloromethane in the third reaction mixture with acetonitrile. In some embodiments, the replacing comprises removing a quantity of the dichloromethane under reduced pressure to afford the concentrate, and diluting the concentrate with acetonitrile to provide a diluted concentrate. In some embodiments, the process further comprises contacting the diluted concentrate with tert-butylamine to provide a mixture comprising an ion pair of formula (IVa). In some embodiments, the process further comprises contacting the diluted concentrate with tert-butylamine and tetrahydrofuran to provide a mixture comprising an ion pair of formula (IVa). In some embodiments, the mixture is a suspension, and the ion pair of formula (IVa) is a solid in the suspension. In some embodiments, the process further comprises filtering the suspension to provide a retentate comprising the ion pair of formula (IVa).

### Deprotection and Salt Formation.

In some embodiments, provided herein is a process comprising contacting a solution with a base, wherein the solution comprises a compound of formula (IIIb):

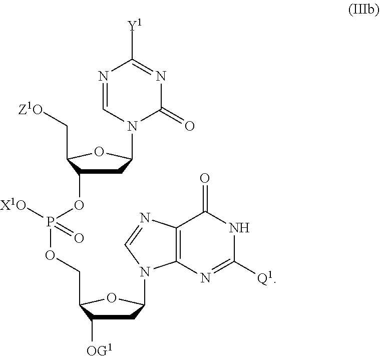

to provide a compound of formula (IVb):

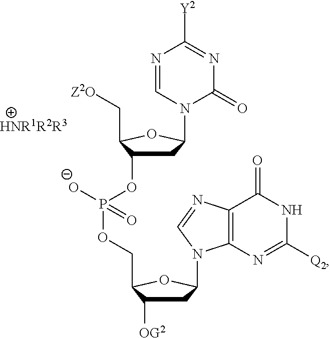

wherein:
the base is NR¹R²R³;
X¹ is 2-cyanoethyl, 2,2,2-trichloroethyl, 2,2,2-tribromoethyl, 2,2,2-trifluoroethyl, benzyl, p-chloroethyl, or p-nitroethyl;
R¹, R², and R³ are each independently H or branched or unbranched alkyl, wherein at least one of R¹, R², and R³ is not H; or R¹ is H or branched or unbranched alkyl and R² and R³ are taken together with the atom to which they are bound to form a ring;
each Z¹, Z², G¹, and G² is independently H or a protecting group;
Y¹ is a protected primary amine; and
each Y² Q¹, and Q² is independently NH₂ or a protected primary amine.

In some embodiments, the solution further comprises acetonitrile. In some embodiments, the solution further comprises acetonitrile and tetrahydrofuran. In some embodiments, the solution comprises no more than 1% (w/w) dichloromethane. In some embodiments, the base comprises tert-butylamine.

In some embodiments, each Z¹ and G¹ is independently H, substituted or unsubstituted acetyl, substituted or unsubstituted phenoxyacetyl, substituted or unsubstituted ethoxymethyl, substituted or unsubstituted benzoyl, or a silyl protecting group. In some embodiments, Q¹ is NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or a formamidine group. In some embodiments, Y¹ is a primary amine protected with substituted or unsubstituted acetyl, substituted or unsubstituted phenoxyacetyl, substituted or unsubstituted ethoxymethyl, substituted or unsubstituted benzoyl, a silyl protecting group, or a formamidine group.

In some embodiments, each Z² and G² is independently H, substituted or unsubstituted acetyl, substituted or unsubstituted phenoxyacetyl, substituted or unsubstituted ethoxymethyl, substituted or unsubstituted benzoyl, or a silyl protecting group. In some embodiments, each Y² and Q² is independently NH₂, or a primary amine protected with substituted or unsubstituted acetyl, substituted or unsubstituted phenoxyacetyl, substituted or unsubstituted ethoxymethyl, substituted or unsubstituted benzoyl, or a silyl protecting group.

In some embodiments, each Z¹ and G¹ is independently H, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, Q¹ is NH₂, or a primary amine protected with ═CHN(CH3)₂, benzoyl, acetyl, isobutyryl, Pac, Tac, iPr-Pac, [(triisopropylsilyl)oxy]methyl, tert-butyldimethylsilyl, or 2′-cyanoethoxymethyl.

In some embodiments, Y¹ is a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, each Z² and G² is independently H, benzoyl, acetyl, isobutyryl, Pac, Tac, iPr-Pac, [(triisopropylsilyl)oxy]methyl, tert-butyldimethylsilyl, or 2′-cyanoethoxymethyl. In some embodiments, each Y² and Q² is independently NH₂, or a primary amine protected with benzoyl, acetyl, isobutyryl, Pac, Tac, iPr-Pac, [(triisopropylsilyl)oxy]methyl, tert-butyldimethylsilyl, or 2′-cyanoethoxymethyl.

In some embodiments, each Z¹ and G¹ is independently H, acetyl, Pac, Tac, or iPr-Pac. In some embodiments, Q¹ is NH₂, or a primary amine protected with ═CHN(CH₃)₂, acetyl, Pac, Tac, or iPr-Pac. In some embodiments, Y¹ is a primary amine protected with ═CHN(CH₃)₂, acetyl, Pac, Tac, or iPr-Pac. In some embodiments, each Z² and G² is independently H, acetyl, Pac, Tac, or iPr-Pac. In some embodiments, each Y² and Q² is independently NH₂, or a primary amine protected with acetyl, Pac, Tac, or iPr-Pac.

In some embodiments, Z¹ is H or acetyl. In some embodiments, G¹ is H or Tac. In some embodiments, Z² is H or acetyl. In some embodiments, G² is H or Tac. In some embodiments, Y¹ is N═CHN(CH₃)₂ or NH(Tac). In some embodiments, Q¹ is NH₂ or NH(Tac). In some embodiments, Y² is NH₂ or NH(Tac). In some embodiments, Q² is NH₂ or NH(Tac). In some embodiments, X¹ is 2-cyanoethyl. In some embodiments, R¹ and R² are each H, and R³ is tert-butyl.

In some embodiments, Y¹ and Q¹ are each independently N═CN(CH₃)₂, NHR^{A}, NHR^{B}, or NH₂, wherein:

if Y¹ is N═C(CH₃)₂ or NH₂, then Y² is NH₂;

if Q¹ is N═C(CH₃)₂ or NH₂, then Q² is NH₂;

if Y¹ is NHR^{A}, then Y² is NHR^{A};

if Y¹ is NHR^{B}, then Y² is NHR^{B};

if Q¹ is NHR^{A}, then Q² is NHR^{A};

if Q¹ is NHR^{B}, then Q² is NHR^{B}; and

Z¹ and G¹ are each independently H, R^{C}, or R^{D}, wherein:

if Z¹ is H, then Z² is H;

if is H, then G² is H;

if Z¹ is R^{C}, then Z² is R^{C};

if Z¹ is R^{D}, then Z² is R^{D};

if is R^{C}, then G² is R^{C};

if G¹ is R^{D}, then G² is R^{D}; and

each R^{A}, R^{B}, R^{C}, and R^{D} is acetyl, Pac, Tac, or iPr-Pac.

### Preparation of Free Acid.

In some embodiments, provided herein is a process comprising:

(i) contacting a solution with a base, wherein the solution comprises a first compound of formula (IV):

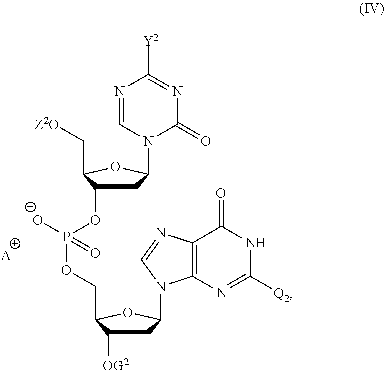

to provide a reaction mixture; and
(ii) contacting the reaction mixture with an acid to provide a second reaction mixture, wherein the second reaction mixture comprises a second compound, wherein the second compound is:

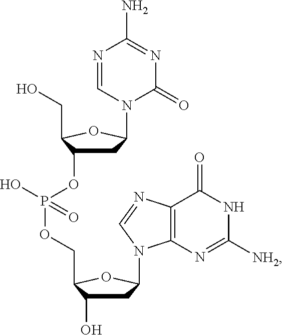

wherein:
A⁺ is an alkylammonium cation;
Z² and G² are each independently H, substituted or unsubstituted acetyl, or substituted or unsubstituted phenoxyacetyl; and
Y² and Q² are each independently NH₂, or a primary amine protected with substituted or unsubstituted acetyl, or substituted or unsubstituted phenoxyacetyl.

In some embodiments, A⁺ is ⁺HNR¹R²R³, wherein R¹, R², and R³ are each independently H or branched or unbranched alkyl, wherein at least one of R¹, R², and R³ is not H; or R¹ is H or branched or unbranched alkyl and R² and R³ are taken together with the atom to which R² and R³ are bound to form a ring. In some embodiments, A⁺ is a tert-butylammonium cation.

In some embodiments, Z² and G² are each independently H, acetyl, Pac, Tac, or iPr-Pac. In some embodiments, Y² and Q² are each independently acetyl, NHAc, NHPac, iPrNHPac, NHTac, or NH₂.

In some embodiments, the first compound is:

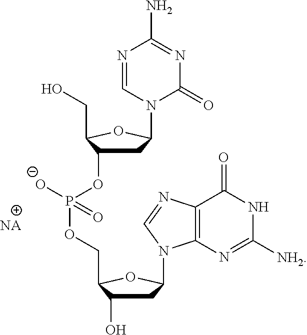

In some embodiments, the solution further comprises a C₁₋₆ alcohol. In some embodiments, the solution further comprises methanol or ethanol. In some embodiments, the solution further comprises methanol. In some embodiments, the base is an alkoxide. In some embodiments, the base is a sodium alkoxide. In some embodiments, the base is sodium methoxide. In some embodiments, the acid is a carboxylic acid. In some embodiments, the acid is formic acid. In some embodiments, the acid is acetic acid.

In some embodiments, the process further comprises removing methanol from the second reaction mixture via distillation to provide a concentrate. In some embodiments, the process further comprises adding DMSO to the concentrate to provide a second solution. In some embodiments, the process further comprises adding DMSO and methanol to the concentrate to provide a second solution. In some embodiments, the process further comprises filtering the second solution to provide a filtrate, and cooling the filtrate to a temperature no more than about 20° C.

In some embodiments, the process further comprises contacting a sodium cation source with the filtrate to provide a third solution, wherein the third solution comprises an ion pair, wherein the ion pair is:

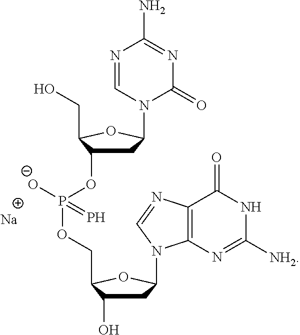

In some embodiments, the sodium cation source is a sodium carboxylate. In some embodiments, the sodium cation source is sodium acetate.

In some embodiments, the process further comprises contacting the third solution with an antisolvent with respect to the ion pair of formula (IIIb) to provide a mixture comprising a solid, the solid comprising the ion pair of formula (IIIb). In some embodiments, the antisolvent is a C1-C6 alcohol. In some embodiments, the antisolvent is ethanol. In some embodiments, the antisolvent is isopropanol. In some embodiments, the process further comprises filtering the mixture to provide a retentate, wherein the retentate comprises the ion pair of formula (IIIb). In some embodiments, the process further comprises washing the retentate with ethanol.

In some embodiments, the reaction mixture comprises no more than 0.3% (a/a), no more than 0.2% (a/a), or no more than 0.1% (a/a) of a third compound as determined by HPLC, wherein the third compound is:

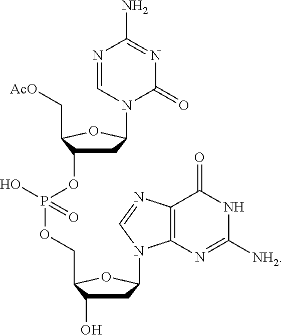

In some embodiments, the reaction mixture comprises from about 0.01% to about 0.3% (a/a), 0.05% to about 0.3% (a/a), 0.01% to about 0.3% (a/a), 0.1% to about 0.3% (a/a), 0.01% to about 0.2% (a/a), or 0.01% to about 0.1% (a/a) of the third compound.

In some embodiments, the reaction mixture comprises at least about 99% (a/a), at least about 99.5% (a/a), at least about 99.6% (a/a), at least about 99.7% (a/a), at least about 99.8% (a/a), or at least about 99% (a/a) of the compound of formula (Ma) as determined by HPLC. In some embodiments, the reaction mixture comprises from about 99.5% to about 99.99% (a/a), from about 99.6% to about 99.99% (a/a), from about 99.7% to about 99.99% (a/a), from about 99.8% to about 99.99% (a/a), or from about 99.9% to about 99.99% (a/a) of the second compound as determined by HPLC.

Preparation of Guadecitabine Sodium from Compound 7.

In some embodiments, the present disclosure provides a process for preparing guadecitabine sodium, comprising:

a) removing the 2-cyanoethyl and N,N-dimethylformamidine groups of Compound 7:

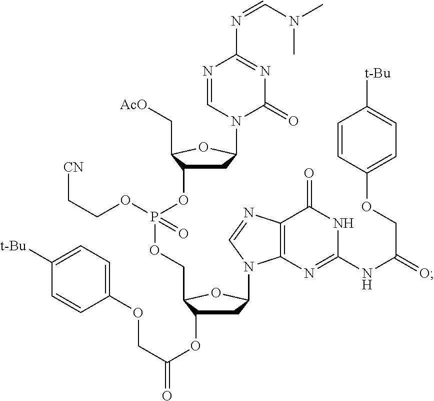

with tert-butylamine to form Compound 8:

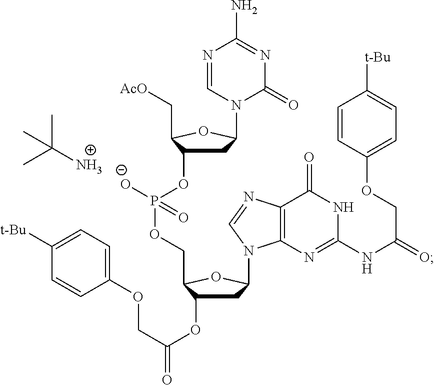

b) removing the acetyl and (4-tertbuylphenoxy)acetyl groups of Compound 8 to form a deprotected adduct, and protonating the deprotected adduct to form Compound 9:

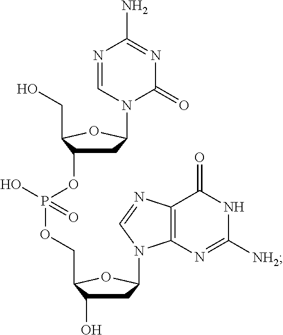

and
c) contacting Compound 9 with a sodium cation source and a base to form Compound 10.

In some embodiments, the removing the 2-cyanoethyl and N,N-dimethylformamidine groups of Compound 7 comprises contacting the tert-butylamine with a mixture, wherein the mixture comprises Compound 7 and a polar organic solvent. In some embodiments, the mixture comprises Compound 7 and acetonitrile. In some embodiments, the mixture further comprises tetrahydrofuran.

In some embodiments, the removing the acetyl and (4-tertbuylphenoxy)acetyl groups of Compound 8 comprises contacting Compound 8 with a base. In some embodiments, the base is an alkoxide. In some embodiments, the base is sodium methoxide.

In some embodiments, the removing the acetyl and (4-tertbuylphenoxy)acetyl groups of Compound 8 comprises contacting a base with a mixture comprising Compound 8 and a polar organic solvent. In some embodiments, the base is an alkoxide. In some embodiments, the base is sodium methoxide. In some embodiments, the polar organic solvent comprises methanol.

In some embodiments, the protonating the deprotected adduct to form Compound 9 comprises contacting the deprotected adduct with an acid. In some embodiments, the acid comprises a carboxylic acid. In some embodiments, the acid is acetic acid.

In some embodiments, the contacting Compound 9 with the sodium cation source to form Compound 10 comprises contacting the sodium cation source with a second mixture, wherein the second mixture comprises Compound 9 and a solvent.

In some embodiments, the contacting Compound 9 with the sodium cation source to form Compound 10 comprises contacting the sodium cation source with a second mixture, wherein the second mixture comprises Compound 9 and methanol. In some embodiments, the second mixture further comprises dimethyl sulfoxide. In some embodiments, the sodium cation source is a sodium carboxylate or sodium alkoxide. In some embodiments, the sodium cation source is an aqueous sodium carboxylate or an aqueous sodium alkoxide. In some embodiments, the sodium cation source is sodium acetate. In some embodiments, the sodium cation source is aqueous sodium acetate.

### Polymorphs.

### Form A.

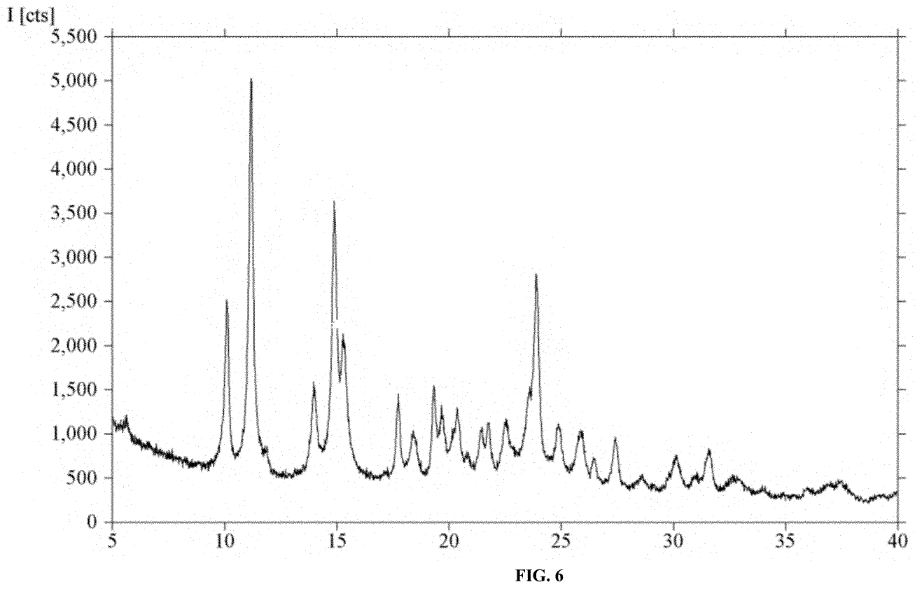

In some embodiments, the disclosure provides a composition comprising a solid form of Compound 10, wherein the solid form exhibits an X-ray powder diffraction pattern substantially the same as the X-ray powder diffraction pattern shown in FIG. 6.

In some embodiments, the disclosure provides a composition comprising a solid form of Compound 10, wherein the solid form exhibits an X-ray powder diffraction pattern that comprises peaks at 10.1°, 11.2°, and 14°±0.2 2θ as measured by X-ray powder diffraction using Cu K alpha radiation. In some embodiments, the X-ray powder diffraction pattern further comprises peaks at 15.3°, 17.7°, and 18.4°±0.2 2θ as measured by X-ray powder diffraction using Cu K alpha radiation. In some embodiments, the X-ray powder diffraction pattern further comprises peaks at 14.9°, 17.7°, and 19.3°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 10.1°, 11.2°, and 14.9°±0.2 2θ as measured by X-ray powder diffraction using Cu K alpha radiation. In some embodiments, the X-ray powder diffraction pattern further comprises peaks at 14°, 15.3°, and 17.7°±0.2 2θ as measured by X-ray powder diffraction using Cu K alpha radiation. In some embodiments, the X-ray powder diffraction pattern further comprises peaks at 15.3°, 17.7°, and 19.3°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 10.1°, 14°, and 15.3°±0.2 2θ as measured by X-ray powder diffraction using Cu K alpha radiation. In some embodiments, the X-ray powder diffraction pattern further comprises peaks at 11.2°, 18.4°, and 19.3°±0.2 2θ as measured by X-ray powder diffraction using Cu K alpha radiation. In some embodiments, the X-ray powder diffraction pattern further comprises peaks at 14.9°, 17.7°, and 23.9°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 11.2°, 14°, and 17.7°±0.2 2θ as measured by X-ray powder diffraction using Cu K alpha radiation. In some embodiments, the X-ray powder diffraction pattern further comprises peaks at 14.9°, 15.3°, and 19.3°±0.2 2θ as measured by X-ray powder diffraction using Cu K alpha radiation. In some embodiments, the X-ray powder diffraction pattern further comprises peaks at 10.1°, 14.9°, and 23.9°±0.2 2θ as measured by X-ray powder diffraction using Cu K alpha radiation.

### Form B.

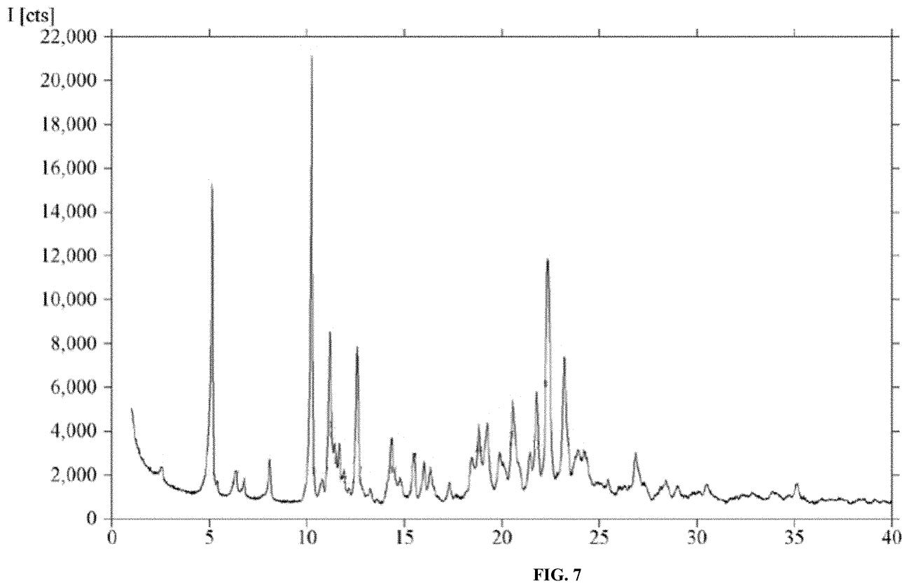

In some embodiments, the disclosure provides a composition comprising a solid form of Compound 10, wherein the solid form exhibits an X-ray powder diffraction pattern substantially the same as the X-ray powder diffraction pattern shown in FIG. 7.

In some embodiments, the disclosure provides a composition comprising a solid form of Compound 10, wherein the solid form exhibits an X-ray powder diffraction pattern that comprises peaks at 5.1°, 10.2°, and 11.2°±0.2 2θ as measured by X-ray powder diffraction using Cu K alpha radiation. In some embodiments, the X-ray powder diffraction pattern further comprises peaks at 6.3°, 8.1°, and 12.6°±0.2 2θ as measured by X-ray powder diffraction using Cu K alpha radiation. In some embodiments, the X-ray powder diffraction pattern further comprises peaks at 12.6°, 14.3°, and 15.5°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 8.1°, 11.7°, and 12.6°±0.2 2θ as measured by X-ray powder diffraction using Cu K alpha radiation. In some embodiments, the X-ray powder diffraction pattern further comprises peaks at 6.3°, 14.3°, and 15.5°±0.2 2θ as measured by X-ray powder diffraction using Cu K alpha radiation. In some embodiments, the X-ray powder diffraction pattern further comprises peaks at 5.1°, 10.2°, and 14.3°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 5.1°, 6.3°, and 8.1°±0.2 2θ as measured by X-ray powder diffraction using Cu K alpha radiation. In some embodiments, the X-ray powder diffraction pattern further comprises peaks at 10.2°, 11.2°, and 12.6°±0.2 2θ as measured by X-ray powder diffraction using Cu K alpha radiation. In some embodiments, the X-ray powder diffraction pattern further comprises peaks at 14.3°, 15.5°, and 20.6°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 5.2°, 10.2°, and 12.6°±0.2 2θ as measured by X-ray powder diffraction using Cu K alpha radiation. In some embodiments, the X-ray powder diffraction pattern further comprises peaks at 6.3°, 8.1°, and 11.2°±0.2 2θ as measured by X-ray powder diffraction using Cu K alpha radiation. In some embodiments, the X-ray powder diffraction pattern further comprises peaks at 11.2°, 14.3°, and 15.5°±0.2 2θ as measured by X-ray powder diffraction using Cu K alpha radiation.

### Form C.

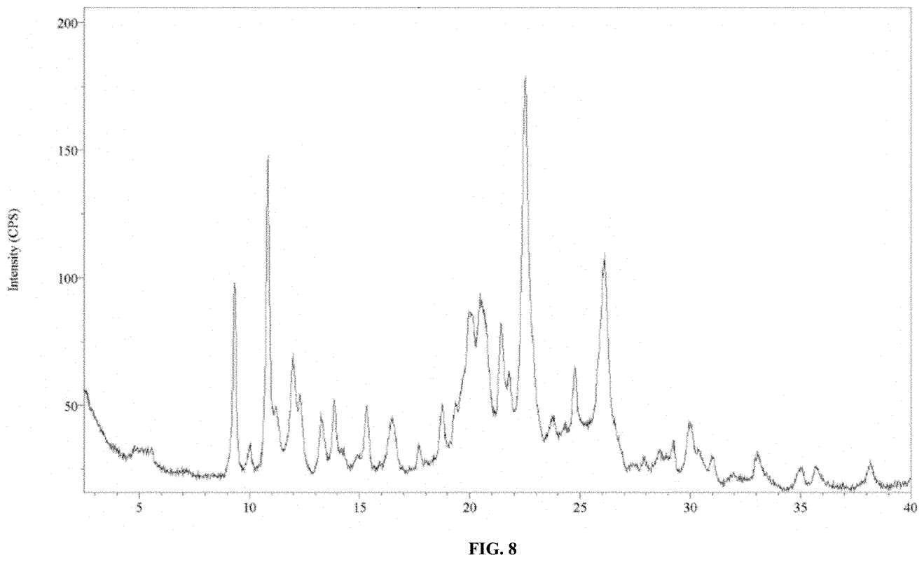

In some embodiments, the disclosure provides a composition comprising a solid form of Compound 10, wherein the solid form exhibits an X-ray powder diffraction pattern substantially the same as the X-ray powder diffraction pattern shown in FIG. 8.

In some embodiments, the disclosure provides a composition comprising a solid form of Compound 10, wherein the solid form exhibits an X-ray powder diffraction pattern that comprises peaks at 9.3°, 10.8°, and 11.7°±0.2 2θ as measured by X-ray powder diffraction using Cu K alpha radiation. In some embodiments, the X-ray powder diffraction pattern further comprises peaks at 13.3°, 13.9°, and 15.4°±0.2 2θ as measured by X-ray powder diffraction using Cu K alpha radiation. In some embodiments, the X-ray powder diffraction pattern further comprises peaks at 10°, 12, and 12.3°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 13.3°, 13.9°, and 15.4°±0.2 2θ as measured by X-ray powder diffraction using Cu K alpha radiation. In some embodiments, the X-ray powder diffraction pattern further comprises peaks at 9.3°, 10.8°, and 12°±0.2 2θ as measured by X-ray powder diffraction using Cu K alpha radiation. In some embodiments, the X-ray powder diffraction pattern further comprises peaks at 9.3°, 11.7°, and 22.5°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 9.3°, 10.8°, and 22.5°±0.2 2θ as measured by X-ray powder diffraction using Cu K alpha radiation. In some embodiments, the X-ray powder diffraction pattern further comprises peaks at 11.7°, 13.3°, and 15.4°±0.2 2θ as measured by X-ray powder diffraction using Cu K alpha radiation. In some embodiments, the X-ray powder diffraction pattern further comprises peaks at 12°, 13.9°, and 16.4°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 10°, 15.4°, and 16.4°±0.2 2θ as measured by X-ray powder diffraction using Cu K alpha radiation. In some embodiments, the X-ray powder diffraction pattern further comprises peaks at 11.7°, 13.3°, and 15.4°±0.2 2θ as measured by X-ray powder diffraction using Cu K alpha radiation. In some embodiments, the X-ray powder diffraction pattern further comprises peaks at 9.3°, 10.8°, and 12.3°±0.2 2θ as measured by X-ray powder diffraction using Cu K alpha radiation.

### Form D.

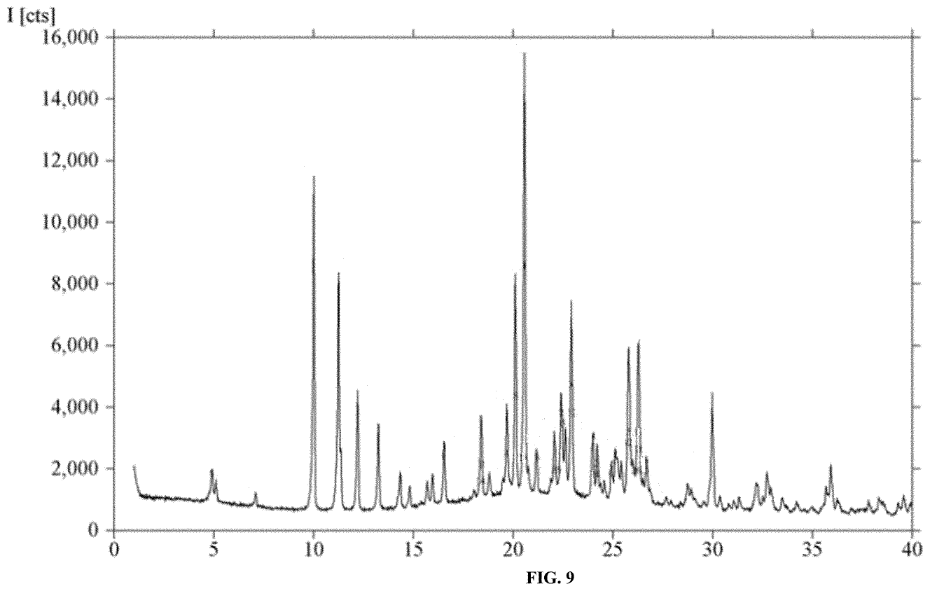

In some embodiments, the disclosure provides a composition comprising a solid form of Compound 10, wherein the solid form exhibits an X-ray powder diffraction pattern substantially the same as the X-ray powder diffraction pattern shown in FIG. 9.

In some embodiments, the disclosure provides a composition comprising a solid form of Compound 10, wherein the solid form exhibits an X-ray powder diffraction pattern that comprises peaks at 10°, 11.2°, and 12.2°±0.2 2θ as measured by X-ray powder diffraction using Cu K alpha radiation. In some embodiments, the X-ray powder diffraction pattern further comprises peaks at 11.4°, 13.2°, and 14.3°±0.2 2θ as measured by X-ray powder diffraction using Cu K alpha radiation. In some embodiments, the X-ray powder diffraction pattern further comprises peaks at 4.9°, 16.5°, and 18.4°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 4.9°, 14.3°, and 16.5°±0.2 2θ as measured by X-ray powder diffraction using Cu K alpha radiation. In some embodiments, the X-ray powder diffraction pattern further comprises peaks at 11.2°, 13.2°, and 18.4°±0.2 2θ as measured by X-ray powder diffraction using Cu K alpha radiation. In some embodiments, the X-ray powder diffraction pattern further comprises peaks at 13.2°, 20.1°, and 20.6°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 11.2°, 12.2°, and 13.2°±0.2 2θ as measured by X-ray powder diffraction using Cu K alpha radiation. In some embodiments, the X-ray powder diffraction pattern further comprises peaks at 4.9°, 14.3°, and 16.5°±0.2 2θ as measured by X-ray powder diffraction using Cu K alpha radiation. In some embodiments, the X-ray powder diffraction pattern further comprises peaks at 7.1°, 20.1°, and 20.6°±0.2 2θ as measured by X-ray powder diffraction using Cu K alpha radiation.

In some embodiments, the disclosure provides a composition comprising a solid form of Compound 10, wherein the solid form exhibits an X-ray powder diffraction pattern that comprises peaks at 13.2°, 16.5°, and 18.4°±0.2 2θ as measured by X-ray powder diffraction using Cu K alpha radiation. In some embodiments, the X-ray powder diffraction pattern further comprises peaks at 10°, 11.2°, and 12.2°±0.2 2θ as measured by X-ray powder diffraction using Cu K alpha radiation. In some embodiments, the X-ray powder diffraction pattern further comprises peaks at 11.2°, 16.5°, and 20.1°±0.2 2θ as measured by X-ray powder diffraction using Cu K alpha radiation.

### Polymorph Production.

In some embodiments, the disclosure provides a process for producing a polymorph of Compound 10, comprising:
(i) mixing Compound 10 with ethanol to provide a suspension;
(ii) filtering the suspension to provide a retentate; and
(iii) drying the retentate under reduced pressure to provide the polymorph,
wherein:
   the ethanol has a water content that is no more than 7% (w/w).

In some embodiments, the reduced pressure is no more than 200 mbar. In some embodiments, the reduced pressure is no more than 150 mbar. In some embodiments, the reduced pressure is no more than 100 mbar. In some embodiments, the reduced pressure is from about 0.1 mbar to about 200 mbar, from about 0.1 mbar to about 150 mbar, or from about 0.1 mbar to about 100 mbar. In some embodiments, the drying under reduced pressure further comprises heating at a temperature of at least 40° C. In some embodiments, the ethanol has a water content that is no more than 5% (w/w).

In some embodiments, the retentate has an X-ray powder diffraction pattern that comprises peaks at 4.9°, 7.2°, and 10.0°±0.2 2θ as measured by X-ray powder diffraction using Cu K alpha radiation. In some embodiments, the X-ray powder diffraction pattern further comprises peaks at 11.3°, 11.5°, and 12.2°±0.2 2θ as measured by X-ray powder diffraction using Cu K alpha radiation. In some embodiments, the X-ray powder diffraction pattern is substantially the same as the X-ray powder diffraction pattern shown in FIG. 8.

In some embodiments, the polymorph has an X-ray powder diffraction pattern that comprises peaks at 10.1°, 11.2°, and 14°±0.2 2θ as measured by X-ray powder diffraction using Cu K alpha radiation. In some embodiments, the X-ray powder diffraction pattern of the polymorph further comprises peaks at 15.3°, 17.7°, and 18.4°±0.2 2θ as measured by X-ray powder diffraction using Cu K alpha radiation.

In some embodiments, the disclosure provides a process for producing a polymorph of Compound 10, comprising drying under reduced pressure a solid form of Compound 10 to provide the polymorph, wherein the solid form of Compound 10 has an X-ray powder diffraction pattern that comprises peaks at 4.9°, 7.2°, and 10.0°±0.2 2θ as measured by X-ray powder diffraction using Cu K alpha radiation. In some embodiments, the solid form exhibits an X-ray powder diffraction pattern substantially the same as the X-ray powder diffraction pattern shown in FIG. 8. In some embodiments, the X-ray powder diffraction pattern of the solid form further comprises peaks at 11.3°, 11.5°, and 12.2°±0.2 2θ as measured by X-ray powder diffraction using Cu K alpha radiation.

In some embodiments, the polymorph has an X-ray powder diffraction pattern that comprises peaks at 10.1°, 11.2°, and 14°±0.2 2θ as measured by X-ray powder diffraction using Cu K alpha radiation. In some embodiments, the X-ray powder diffraction pattern of the polymorph further comprises peaks at 15.3°, 17.7°, and 18.4°±0.2 2θ as measured by X-ray powder diffraction using Cu K alpha radiation. In some embodiments, the polymorph exhibits an X-ray powder diffraction pattern substantially the same as the X-ray powder diffraction pattern shown in FIG. 6.

In some embodiments, the reduced pressure is no more than 200 mbar. In some embodiments, the reduced pressure is no more than 150 mbar. In some embodiments, the reduced pressure is no more than 100 mbar. In some embodiments, the drying under reduced pressure further comprises heating at a temperature of at least 40° C.

In some embodiments, the disclosure provides a process for preparing Compound 10, comprising contacting a first mixture with a sodium cation source to provide a second mixture, wherein the first mixture comprises Compound 9 and dimethyl sulfoxide, and the second mixture comprises Compound 10.

In some embodiments, the sodium cation source is an aqueous sodium cation source. In some embodiments, the sodium cation source comprises sodium acetate. In some embodiments, the first mixture further comprises methanol.

In some embodiments, the process further comprises isolating Compound 10 from the second mixture to provide a solid that comprises Compound 10. In some embodiments, the isolating Compound 10 comprises contacting the second mixture with an antisolvent to provide a precipitate, and isolating the precipitate by filtration to provide the solid.

In some embodiments, the solid has a purity of at least about 95%, about 96%, about 97, about 98%, or about 99% (a/a) as determined by HPLC. In some embodiments, the solid has a purity from about 95% to about 99.9% (a/a), from about 96% to about 99.9% (a/a), from about 97% to about 99.9% (a/a), from about 98% to about 99.9% (a/a), from about 99% to about 99.9% (a/a), from about 95% to about 99.5% (a/a), from about 96% to about 99.5% (a/a), from about 97% to about 99.5% (a/a), from about 98% to about 99.5% (a/a), or from about 99% to about 99.5% (a/a) as determined by HPLC.

In some embodiments, the antisolvent is a C₂-C₆ alcohol. In some embodiments, the antisolvent comprises ethanol or isopropanol, or mixtures thereof. In some embodiments, the antisolvent comprises ethanol. In some embodiments, the antisolvent comprises isopropanol. In some embodiments, the antisolvent is ethanol. In some embodiments, the antisolvent is isopropanol.

In some embodiments, the contacting the second mixture with the antisolvent is conducted at a temperature no more than about 15° C. In some embodiments, the contacting the second mixture with the antisolvent is conducted at a temperature from about −5° C. to about 15° C., from about 0° C. to about 15° C., from about −5° C. to about 10° C., from about −5° C. to about 5° C., or from about 0° C. to about 5° C. In some embodiments, the contacting the first mixture with the sodium cation source is conducted at a temperature no more than about 15° C. In some embodiments, the contacting the first mixture with the sodium cation source is conducted at a temperature from about −5° C. to about 15° C., from about 0° C. to about 15° C., from about −5° C. to about 10° C., from about −5° C. to about 5° C., or from about 0° C. to about 5° C. In some embodiments, the solid is amorphous.

In some embodiments, the contacting the second mixture with the antisolvent is conducted at a temperature no more than about 30° C., no more than about 29° C., no more than about 28° C., no more than about 27° C., no more than about 26° C., or no more than about 25° C. In some embodiments, the contacting the second mixture with the antisolvent is conducted at a temperature from about 5° C. to about 25° C., from about 10° C. to about 25° C., from about 15° C. to about 25° C., or from about 20° C. to about 25° C. In some embodiments, the contacting the first mixture with the sodium cation source is conducted at a temperature no more than about 35° C., no more than about 34° C., no more than about 33° C., no more than about 32° C., no more than about 31° C., or no more than about 30° C. In some embodiments, the contacting the second mixture with the antisolvent is conducted at a temperature from about 15° C. to about 35° C., from about 20° C. to about 35° C., from about 25° C. to about 35° C., from about 10° C. to about 35° C., from about 10° C. to about 30° C., from about 15° C. to about 30° C., from about 20° C. to about 30° C., or from about 25° C. to about 30° C. In some embodiments, the solid exhibits an X-ray powder diffraction pattern substantially the same as the X-ray powder diffraction pattern shown in FIG. 9.

In some embodiments, the solid exhibits an X-ray powder diffraction pattern that comprises peaks at 10°, 11.2°, and 12.2°±0.2 2θ as measured by X-ray powder diffraction using Cu K alpha radiation. In some embodiments, the X-ray powder diffraction pattern further comprises peaks at 11.4°, 13.2°, and 14.3°±0.2 2θ as measured by X-ray powder diffraction using Cu K alpha radiation. In some embodiments, the X-ray powder diffraction pattern further comprises peaks at 4.9°, 16.5°, and 18.4°±0.2 2θ as measured by X-ray powder diffraction using Cu K alpha radiation.

In some embodiments, the disclosure provides a process for producing a polymorph of Compound 10, comprising:
(i) contacting a first mixture with ethanol to provide a second mixture, wherein the first mixture comprises Compound 10 and water;
(ii) cooling the second mixture to provide a precipitate; and
(iii) isolating the precipitate via filtration to provide the polymorph.

In some embodiments, the polymorph has an X-ray powder diffraction pattern that comprises peaks at 4.9°, 7.2°, and 10.0°±0.2 2θ as measured by X-ray powder diffraction using Cu K alpha radiation. In some embodiments, the X-ray powder diffraction pattern further comprises peaks at 11.3°, 11.5°, and 12.2°±0.2 2θ as measured by X-ray powder diffraction using Cu K alpha radiation. In some embodiments, the polymorph exhibits an X-ray powder diffraction pattern substantially the same as the X-ray powder diffraction pattern shown in FIG. 8.

In some embodiments, the process further comprises washing the polymorph with a C₁-C₆ alcohol. In some embodiments, the C₁-C₆ alcohol is ethanol, isopropanol, or a mixture thereof. In some embodiments, the C₁-C₆ alcohol comprises ethanol. In some embodiments, the C₁-C₆ alcohol is ethanol. In some embodiments, the C₁-C₆ alcohol is at a temperature of about 5° C. to about 15° C. during the washing.

In some embodiments, the process further comprises washing the polymorph with ethanol, wherein the ethanol is at a temperature from about 5° C. to about 15° C., from about 0° C. to about 20° C., or from about 5° C. to about 15° C. during the washing.

In some embodiments, the process further comprises:
(i) drying the polymorph under reduced pressure at a temperature from about 0° C. to about 30° C., from about 0° C. to about 35° C., from about 5° C. to about 30° C., from about 10° C. to about 30° C., from about 15° C. to about 30° C., from about 20° C. to about 30° C., or from about 25° C. to about 30° C.; and then
(ii) drying the polymorph under reduced pressure at a temperature from about 30° C. to about 70° C., from about 0° C. to about 70° C., from about 10° C. to about 70° C., from about 20° C. to about 70° C., from about 30° C. to about 65° C., from about 35° C. to about 70° C., from about 40° C. to about 70° C., from about 45° C. to about 70° C., from about 50° C. to about 70° C., or from about 60° C. to about 70° C.,
   to provide a solid comprising a second polymorph.

In some embodiments, the reduced pressure is no more than 200 mbar. In some embodiments, the reduced pressure is no more than 150 mbar. In some embodiments, the reduced pressure is no more than 100 mbar. In some embodiments, the reduced pressure is from about 0.1 mbar to about 200 mbar, from about 0.1 mbar to about 150 mbar, or from about 0.1 mbar to about 100 mbar.

In some embodiments, the first mixture comprises from about 1% to about 30%, from about 1% to about 20%, from about 1% to about 10%, from about 0.1% to about 10%, from about 1% to about 8%, from about 2% to about 8%, or from about 3% to about 7% (w/w) Compound 10.

In some embodiments, the second mixture comprises from about 60% to about 90%, from about 60% to about 95%, from about 60% to about 98%, from about 70% to about 90%, from about 70% to about 85%, or from about 75% to about 85% (w/w) ethanol.

In some embodiments, the cooling the second mixture comprises cooling the second mixture to a temperature from about 5° C. to about 15° C., from about 0° C. to about 20° C., or from about 0° C. to about 15° C.

In some embodiments, the contacting the first mixture is conducted at a temperature from about 5° C. to about 35° C., from about 10° C. to about 35° C., from about 15° C. to about 35° C., from about 15° C. to about 30° C., from about 15° C. to about 25° C.

In some embodiments, the second polymorph has an X-ray powder diffraction pattern that comprises peaks at 10.1°, 11.2°, and 14°±0.2 2θ as measured by X-ray powder diffraction using Cu K alpha radiation. In some embodiments, the X-ray powder diffraction pattern of the second polymorph further comprises peaks at 15.3°, 17.7°, and 18.4°±0.2 2θ as measured by X-ray powder diffraction using Cu K alpha radiation.

In some embodiments, the disclosure provides a process comprising:
(i) contacting Compound 10 with a first mixture to provide a second mixture, wherein the first mixture comprises a solvent, wherein the solvent is a combination of water and ethanol;
(ii) heating the second mixture to a temperature of from about 30° C. to about 45° C.;
(iii) after the heating, cooling the second mixture to provide a precipitate; and
(iv) isolating the precipitate via filtration to provide a polymorph of Compound 10.

In some embodiments, the polymorph exhibits an X-ray powder diffraction pattern substantially the same as the X-ray powder diffraction pattern shown in FIG. 9. In some embodiments, the polymorph exhibits an X-ray powder diffraction pattern that comprises peaks at 10°, 11.2°, and 12.2°±0.2 2θ as measured by X-ray powder diffraction using Cu K alpha radiation. In some embodiments, the X-ray powder diffraction pattern of the polymorph further comprises peaks at 11.4°, 13.2°, and 14.3°±0.2 2θ as measured by X-ray powder diffraction using Cu K alpha radiation. In some embodiments, the X-ray powder diffraction pattern of the second polymorph further comprises peaks at 4.9°, 16.5°, and 18.4°±0.2 2θ as measured by X-ray powder diffraction using Cu K alpha radiation.

In some embodiments, the heating the second mixture comprises heating the second mixture to a temperature of from about 30° C. to about 40° C.

In some embodiments, the process further comprises washing the polymorph with a C₁-C₆ alcohol, wherein the C₁-C₆ alcohol is at a temperature of about 5° C. to about 15° C. during the washing. In some embodiments, the C₁-C₆ alcohol is ethanol, isopropanol, or a mixture thereof. In some embodiments, the C₁-C₆ alcohol comprises ethanol. In some embodiments, the C₁-C₆ alcohol is ethanol.

In some embodiments, the process further comprises washing the polymorph with ethanol that has a temperature from about 5° C. to about 15° C., from about 0° C. to about 20° C., from about 0° C. to about 15° C., or from about 8° C. to about 12° C.

In some embodiments, the process further comprises, after the washing:
(i) drying the polymorph under reduced pressure at a temperature from about −5° C. to about 25° C., from about 0° C. to about 25° C., from about 0° C. to about 30° C., from about 0° C. to about 35° C., from about 5° C. to about 30° C., from about 10° C. to about 30° C., from about 15° C. to about 30° C., from about 20° C. to about 30° C., or from about 25° C. to about 30° C.; and then
(ii) drying the polymorph under reduced pressure at a temperature from about 30° C. to about 70° C., from about 0° C. to about 60° C., from about 0° C. to about 50° C., from about 0° C. to about 45° C., from about 10° C. to about 50° C., from about 20° C. to about 45° C., from about 30° C. to about 40° C., from about 25° C. to about 70° C., from about 25° C. to about 50° C., or from about 25° C. to about 40° C., to provide a solid comprising a second polymorph.

In some embodiments, the process further comprises drying the polymorph under reduced pressure at a temperature from about 0° C. to about 30° C., and then at a temperature from about 30° C. to about 50° C. under reduced pressure to provide a solid comprising a second polymorph.

In some embodiments, the second polymorph exhibits an X-ray powder diffraction pattern substantially the same as the X-ray powder diffraction pattern shown in FIG. 7. In some embodiments, the second polymorph exhibits an X-ray powder diffraction pattern that comprises peaks at 5.1°, 10.2°, and 11.2°±0.2 2θ as measured by X-ray powder diffraction using Cu K alpha radiation. In some embodiments, the X-ray powder diffraction pattern of the second polymorph further comprises peaks at 6.3°, 8.1°, and 12.6°±0.2 2θ as measured by X-ray powder diffraction using Cu K alpha radiation. In some embodiments, the X-ray powder diffraction pattern of the second polymorph further comprises peaks at 12.6°, 14.3°, and 15.5°±0.2 2θ as measured by X-ray powder diffraction using Cu K alpha radiation.

In some embodiments, the solvent is from about 5% to about 50%, from about 5% to about 40%, from about 5% to about 30%, from about 5% to about 20%, from about 10% to about 50%, from about 10% to about 40%, from about 10% to about 30%, from about 10% to about 25%, from about 10% to about 20%, or from about 15% to about 20% (v/v) water in ethanol.

In some embodiments, the cooling the second mixture comprises cooling the second mixture to a temperature from about 5° C. to about 15° C., from about 0° C. to about 20° C., from about 0° C. to about 15° C., or from about 8° C. to about 12° C.

In some embodiments, the second mixture comprises from about 0.1% to about 10%, 0.5% to about 30%, 0.5% to about 20%, 0.5% to about 10%, 0.5% to about 5%, 0.1% to about 5%, from about 0.1% to about 3%, or from about 0.5% to about 3% (w/w) Compound 10.

### EXAMPLES

### Example 1: Preparation of Compound 2

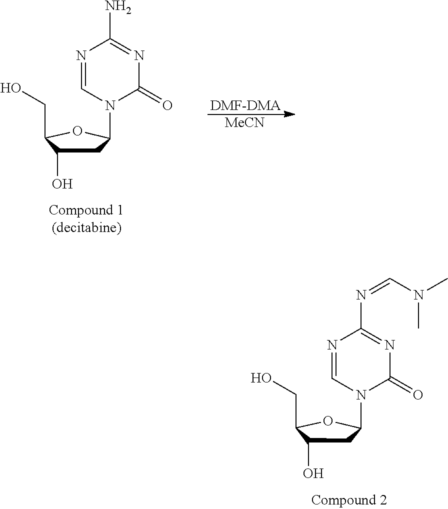

Compound 1 and acetonitrile were added together in an appropriate reactor between 18 and 30° C. and stirred for at least 15 minutes. (Dimethoxymethyl)dimethylamine (DMF-DMA) mixed with acetonitrile was then added at a temperature between 15 and 35° C. The reactor contents was then stirred at this temperature until the reaction reached completion as determined by HPLC (conversion: ≥99.0%). The product precipitated from the reaction mixture and was isolated and washed with acetonitrile. Compound 2 was dried at jacket temperature ≤30° C. until the acetonitrile content was below or equal to 0.5% w/w.

### Example 2: Preparation of Compound 3

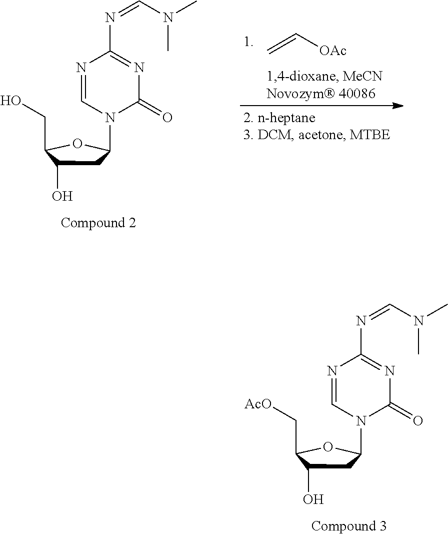

To 1,4-dioxane at 23-28° C. was added Compound 2 and the reactor contents were stirred until a homogeneous suspension was formed. Novozym® 40086 was added to the reactor followed by vinyl acetate over at least 15 minutes at 23-28° C. Vinyl acetate was rinsed in with acetonitrile, and the mixture was then heated to 26-36° C. until the reaction reached completion as determined by HPLC (IPC: conversion: ≥99.7%).

The reactor contents were then filtered over Celite and 1,4-dioxane and acetonitrile were used to wash the filter cake. The contents of the reactor were then cooled to 24 to 34° C. and n-heptane was added over a period of 24-40 minutes. The reactor was further cooled to 14 to 24° C. and stirred at this temperature for 45 minutes to 24 h. The product was isolated by filtration, washed with n-heptane and dried at jacket temperature of ≤25° C. until a free-flowing powder was obtained. The sequence consisting of a clarifying filtration, precipitation, product filtration, and washing may be performed in up to 3 parts, in which case the solvent amounts are to be adjusted accordingly.

The crude product was then dissolved in dichloromethane and stirred at ≤25° C. until a clear solution formed. Acetone was then added. A suspension containing seed crystals of Compound 3 in acetone was then added and the reaction mixture stirred for at least 15 to 45 minutes at ≤25° C. Solvent was then distilled from the reactor under reduced pressure at a jacket temperature of ≤50° C., and the reaction mixture was cooled to 22-32° C. Acetone was then added, followed by methyl tert-butyl ether. The reactor contents were then cooled to 15 to 25° C. and stirred at this temperature for 90 minutes to 24 h. The product was filtered and washed with methyl tert-butyl ether followed by n-heptane, and then dried at ≤30° C. until the amount of residual solvent was below 7% w/w. The product was isolated as a solid.

### Example 3: Preparation of Compound 8

### Synthesis of Compound 4.

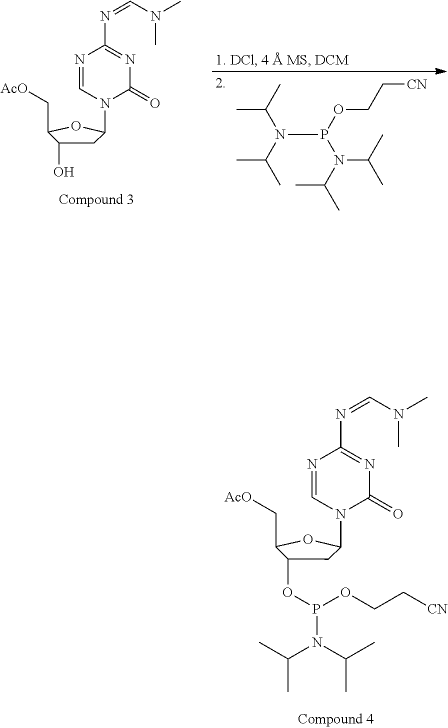 [SMILES: CC(=O)OCC1OC(n2cnc(/N=C\N(C)C)nc2=O)CC1O ; CC(C)N(C(C)C)P(OCCC#N)N(C(C)C)C(C)C ; CC(=O)OCC1OC(n2cnc(/N=C\N(C)C)nc2=O)CC1OP(OCCC#N)N(C(C)C)C(C)C]

Compound 3 was dissolved in dichloromethane in an appropriate reactor between 5 and 25° C. The solution was dried over a bed of 4 Å molecular sieves until the end-point for water content as determined by Karl-Fischer was reached (water content ≤0.01% w/w). Dichloromethane was distilled under reduced pressure at a jacket temperature ≤35° C. The reactor was cooled to between 20° C. and 25° C., and 4,5-dicyanoimidazole was added. The contents of the reactor were cooled to between 10° C. and 25° C. and stirred for at least 15 minutes. The jacket temperature of the reactor was then set to between 5° C. and 15° C., and a solution of 3-((bis(diisopropylamino)phosphanyl)oxy) propanenitrile in DCM was charged to the reactor in under 5 minutes. The reaction mixture was then heated to between 15 and 25° C. until the reaction reached completion as determined by HPLC (conversion: ≥98.5%). The reaction mixture was used directly in the next step.

### Synthesis of Compound 6.

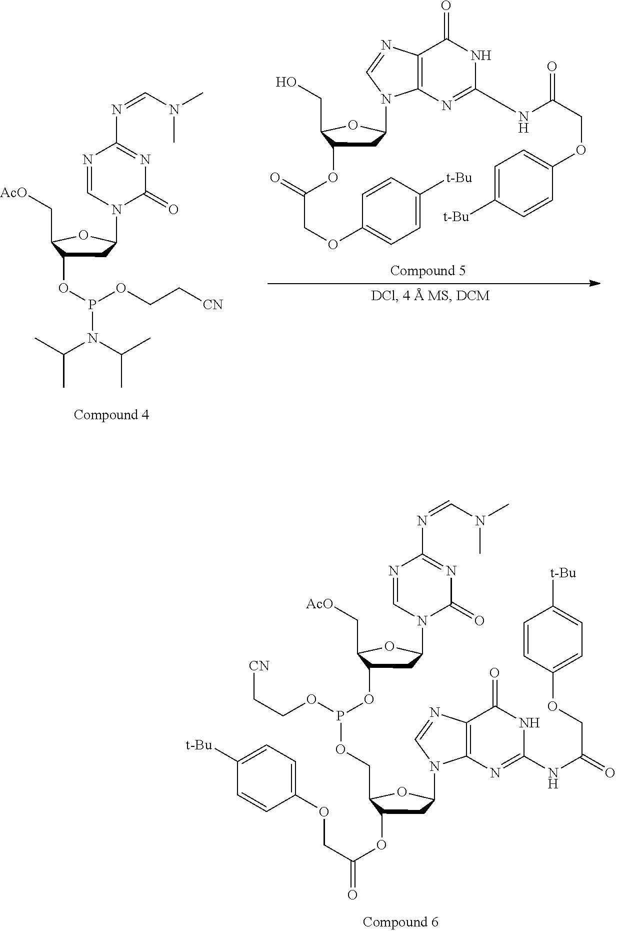

Compound 5 was dissolved in dichloromethane in an appropriate reactor between 5 and 25° C. The resulting solution was dried over a bed of 4 Å molecular sieves until the end-point for water content as determined by Karl-Fischer was reached (water content ≤0.01% w/w). Dichloromethane was distilled off under reduced pressure at a jacket temperature ≤35° C.

The reaction mixture containing Compound 4 from the previous step was cooled to between 5 and 25° C. and stirred for 15 min to 24 h. The solution of Compound 5 in dichloromethane was then charged at ≤25° C. The charging vessel was rinsed with DCM and the rinse was added to the reactor. The batch temperature was set to between 10° C. and 25° C. and DCI was charged. The mixture was agitated at 10 to 25° C. for 2 to 48 h. The contents of the reaction mixture were heated to between 15° C. and 25° C. and agitated until the reaction was complete as determined by HPLC (conversion: ≥98%). The reaction mixture was used directly in the next step of the process without any workup.

### Synthesis of Compound 7.

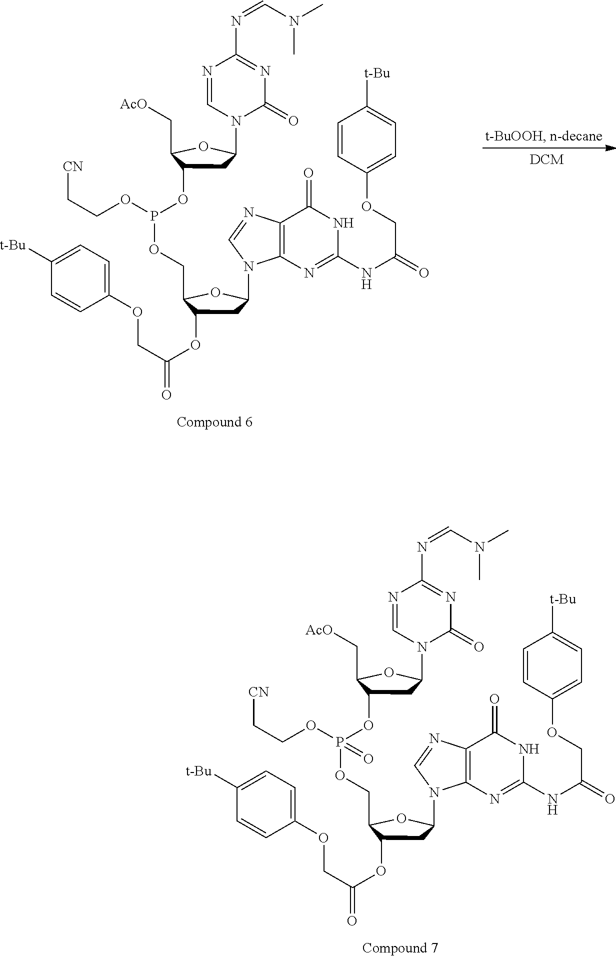

The contents of the reactor from the previous step containing Compound 6 were cooled to between 5 and 17° C., and tert-butyl hydroperoxide in decane was charged. The tert-butyl hydroperoxide container was rinsed with DCM, and the rinse was charged into the reactor. The contents of the reactor were warmed to between 15 and 20° C. for 4-24 h. The reaction was monitored by HPLC until deemed complete (conversion: ≥97%), and the reaction mixture was used directly in the next step of the process.

### Synthesis of Compound 8.

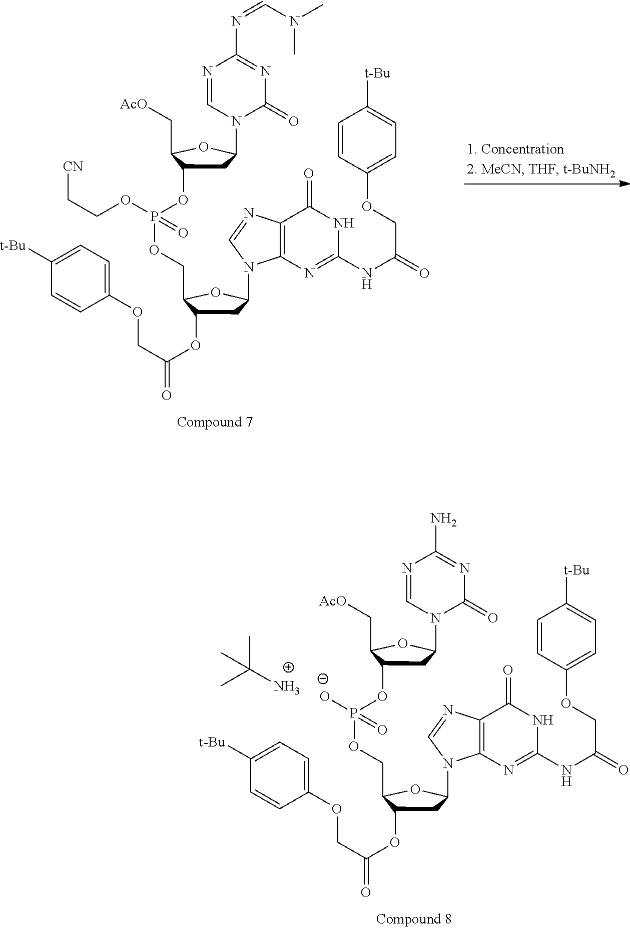

The reaction mixture from the previous step containing Compound 7 was concentrated under reduced pressure at a jacket temperature of ≤35° C. Acetonitrile was then charged to the reactor and distillation was continued under reduced pressure. This cycle was repeated until the target residual DCM content was reached (DCM≤1% w/w). The contents of the reactor were cooled to 10 to 25° C. and acetonitrile was added. Tetrahydrofuran was added to the reactor followed by a solution of tert-butylamine in acetonitrile at a temperature ≤25° C. over a period of 10 to 60 minutes. The reaction mixture was set to 20 to 25° C. and stirred for 25 h to 72 h. The product was filtered at a jacket temperature of 10 to 30° C. The product was washed with a mixture of acetonitrile and tetrahydrofuran and dried at ≤40° C. under reduced pressure until the sum of residual solvents was ≤1.0% w/w).

### Example 4: Preparation of Compound 10

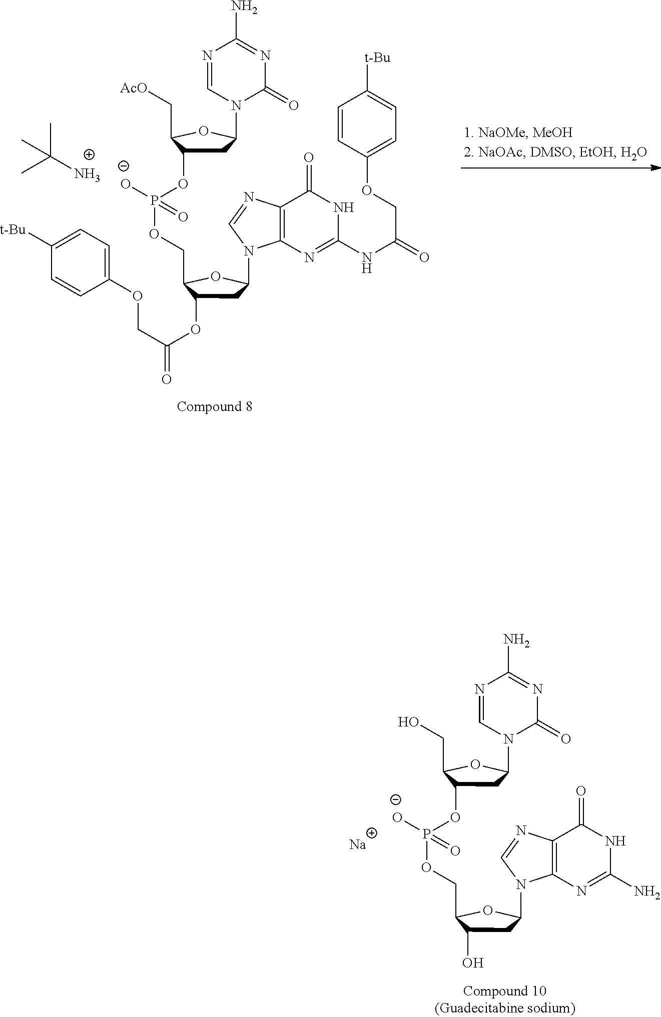

### Process A.

Compound 8 was dissolved in methanol in an appropriate reactor at a jacket temperature of 25° C. The reactor contents were then cooled to 0° C. (range±5° C.) and sodium methylate solution in methanol was added followed by methanol. The reaction was stirred at 0° C. (range±5° C.) until completion as determined by HPLC was attained (conversion≥99.5%, Compound 12≤0.3 area %). Acetic acid was added to quench the reaction followed by methanol. The reaction mixture was then heated at jacket temperature ≤35° C. under reduced pressure until approximately 20% of the methanol was distilled. Dimethyl sulfoxide was then added, and the mixture was heated under reduced pressure until the majority of the remaining methanol was distilled. The reaction temperature was then set to between 5-25° C. and dimethyl sulfoxide was added followed by methanol. The reaction mixture was then filtered and cooled to ≤20° C. Sodium acetate in deionized water was then added at such a rate that the reaction temperature was maintained at ≤30° C. An additional portion of deionized water was used to rinse in any remaining sodium acetate. Ethanol was then added (temperature range 19 to 25° C.), and the batch contents were maintained at a temperature of 20° C. (range≤25° C.). The suspension was isolated by filtration, and the retentate was washed with ethanol and combined with the retentate from a second reaction run in parallel. The combined portions were dried under vacuum at jacket temperature ≤35° C. until a free flowing powder was formed.

### Process B.

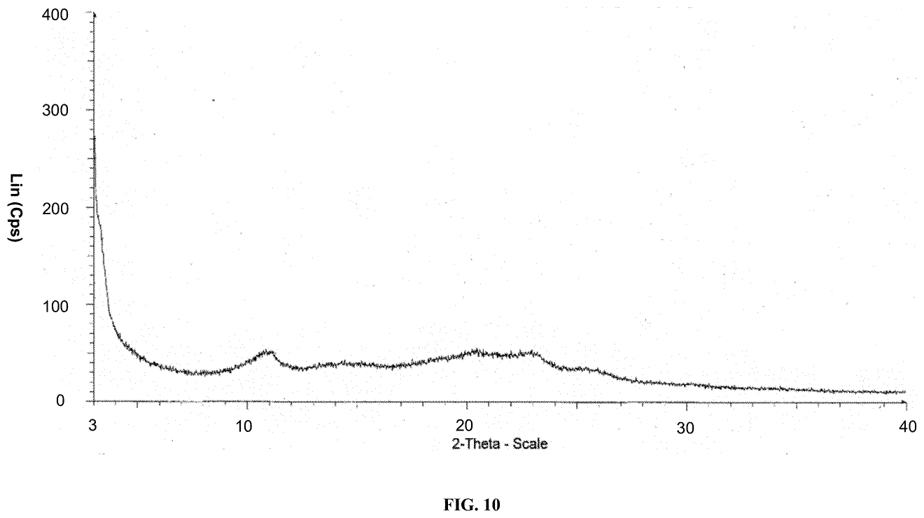

Compound 8 (20.0 g) was dissolved in methanol (approximately 839 mL) in an appropriate reactor at a jacket temperature of 25° C. The reactor contents were then cooled to 0° C. (range±2° C.) and sodium methylate solution in methanol (28% NaOMe, 12.9 mL) was added followed by methanol (approximately 12.9 mL). The reaction was stirred at 0° C. (range±2° C.) until completion as determined by IPC was attained (IPC: conversion≥99.5%, Compound 12≤0.3 area %). Acetic acid (4.2 mL) was added to quench the reaction. The reaction mixture was then heated at jacket temperature ≤35° C. under reduced pressure until approximately 20% of the methanol was distilled. Dimethyl sulfoxide (88.6 mL) was then added, and the mixture was heated under reduced pressure until the majority of the remaining methanol was distilled. The reaction temperature was then set to between 5-25° C. and dimethyl sulfoxide (approximately 29.8 mL) was added followed by methanol (12.6 mL). The reaction mixture was then filtered and cooled to 0-5° C. Sodium acetate (6.8 g) in deionized water (approximately 442.8 mL, cooled to 0-5° C. beforehand) was then added at such a rate that the reaction temperature was maintained at 0-10° C. Ethanol (1447 mL) was then added (temperature 7.5° C.±2° C.), and the batch contents were maintained at a temperature at 5-10° C. The suspension was isolated by filtration, and the retentate was washed with ethanol (approximately 182 mL). Crude Compound 10 was dried under vacuum at a jacket temperature ≤25° C. until a free-flowing powder was obtained (9.39 g of crude Compound 10, 100% crude yield, amorphous, HPLC purity: 99.16%). Analysis of the powder via XRPD revealed that the powder was amorphous (FIG. 10).

### Example 5A: Production of Form A

Crude Compound 10 was added to a reactor containing deionized water at a temperature of 18-22° C. The reaction mixture was stirred for 80-120 minutes and then ethanol was added over a period of at least 5 h while the temperature was maintained between 10-30° C. The reaction was stirred for a further 50-70 minutes at 10-30° C. and then cooled over at least 1 h to 8-12° C. After stirring at this temperature for 110 minutes to 72 h, the product was isolated by filtration with the jacket temperature of the filtration apparatus set to 8-12° C. The product was washed with ethanol that was pre-cooled to a temperature of 8-12° C. The wash liquors were sampled to determine water content. Guadecitabine sodium was dried under vacuum at a jacket temperature ≤25° C. until a free-flowing powder was obtained. Drying was continued at a jacket temperature ≤60° C. to afford the product as a white to off-white solid. The solid consisted predominantly of Form A, which is further described in EXAMPLE 6.

### Example 5B: Production of Form B

Crude Compound 10 (amorphous, 4.0 g, HPLC purity 99.16% from Example 4, Process B) was added to a reactor containing deionized water (58 mL) and ethanol (266 mL) at a temperature of 18-22° C. The reaction mixture was stirred for 90 minutes at 18-22° C., then warmed to 35° C. and stirred at 35° C. for 120 minutes. The reaction was cooled over 1 h to 8-12° C. After stirring at 8-12° C. for 2 h, the product was isolated by filtration and washed with ethanol (75.2 mL) that was pre-cooled to a temperature of 8-12° C. Analysis of the retentate revealed Form D of Compound 10 was obtained, which is further described in EXAMPLE 6. Form D exhibited a greater degree of filterability relative to Form C. The retentate was dried under vacuum at ≤25° C. and then dried under vacuum at ≤45° C. until a free-flowing powder was obtained (3.63 g of Compound 10, 90.8% yield, Form B, HPLC purity 99.40% a/a). Analysis of the dried product via XRPD revealed Form B of Compound 10 was obtained.

### Example 6: Preparation and Characterization of Guadecitabine Sodium Polymorphs

Studies were performed to identify the different polymorphic forms of Compound 10 drug substance. Seven polymorphs of Compound 10 in total have been identified (TABLE 1).

TABLE 1

| Polymorph | Description* |
|---|---|
| Form A | Stable form at 0% RH |
| Form B | Stable form at 0% RH |
| Form C | Formed from ethanolic slurry of Form A |
| Form D | Formed from Form B at 58% RH |
| Form H | Formed from Form B at 33% RH |
| Form K | Formed from Form A at 58% RH |
| Form L | Formed from Form A at 97% RH |

*RH = relative humidity

### Determination of Effect of Crystallization Parameters on Compound 10 Form.

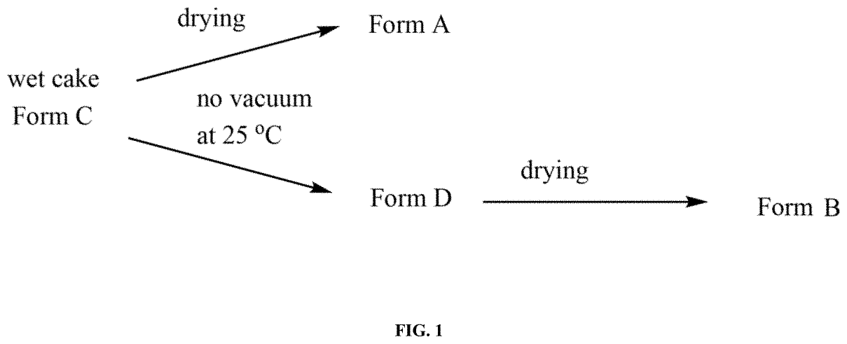

The final crystallization of guadecitabine sodium was assessed using in-situ Raman spectroscopy to understand the potential for formation of different polymorphs. The crystallization uses water and ethanol as solvent and antisolvent respectively, and incorporates a washing step with ethanol to help remove residual water prior to drying. The transitions between polymorphic forms that were observed during this process are described in FIG. 1. The following observations were made:
The wet cake immediately prior to drying is Form C.
Direct drying of Form C under high vacuum leads to Form A.
If the wet cake is not dried immediately, the cake can convert to Form D.
Form D converts to Form B upon drying.

Based on these observations, Form A and Form B are the relevant polymorphic forms after drying.

### Studies to Determine the Effect of Relative Humidity on Polymorph.

Compound 10 is hygroscopic and is therefore packaged and stored at conditions that inhibit moisture uptake. Experiments were performed to understand the effect of relative humidity on various polymorphic forms of Compound 10. Form A and Form B were subjected to increasing relative humidity (TABLE 2) and monitored using XRPD to evaluate polymorphism. This test showed that both polymorphs are stable at 0% RH, which is the relevant condition for storage and further processing.

Form A was stable at 0% RH, and exhibited stability for at least 2 weeks at 33% RH after which time Form A converted to Form K. At a RH of 97% and above, Form K converted to Form L.

Form B was stable at 0% RH and converted to Form H at 33% RH. Increasing RH led to formation of Form D, followed by deliquescence to afford a mesophase above 97% RH.

TABLE 2

| | Polymorph | Polymorph | Polymorph | |
|---|---|---|---|---|
| | observed | observed | observed | |
| Polymorph | at 0% RH | at 33% RH | at 58% RH | 97% RH |
| Form A | A | A | K | L |
| Form B | B | H | D | Mesophase |

### Factors Controlling the Formation of Polymorphs A and B During Reslurry, Filtration, and Drying.

Compound 10 was reslurried in batches of ethanol with varying water content. Slurries were performed at 10-15° C. for 10-15 min for each re-slurry. The solids were then filtered, deliquored, and pre dried at 40 mbar at no more than 25° C. for maximum of 4 hours. Vacuum was then increased to the maximum possible until a free-flowing powder was obtained. The temperature was then increased to no more than 50° C. until drying was complete. The influence of the following parameters on the polymorphs were addressed:
Water content in wet cake after filtration of crystallized material
Temperature and KF of crystallization mixture prior to filtration
Temperature during filtration of material after crystallization
Effect of “vacuum” during the drying protocol

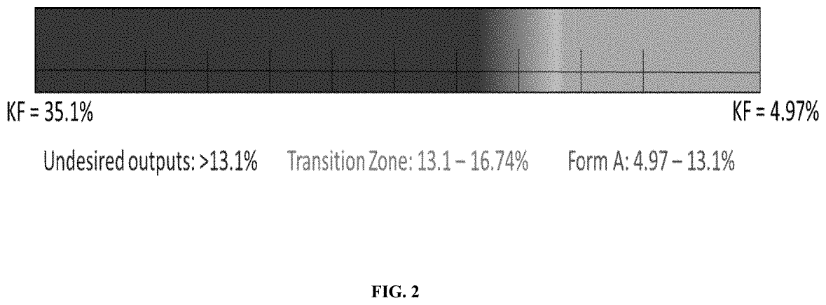

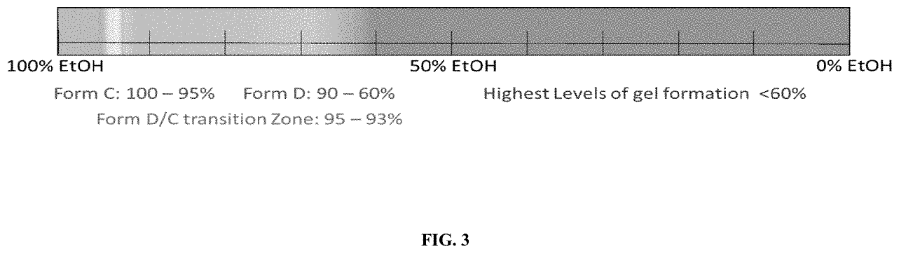

Form C was maintained and isolated from reslurries carried out using >95% EtOH solutions before drying. The effect of water content in the reslurry solvent and wet cake after filtration is summarized in TABLE 3. The effect of isolated wet cake water content as assessed by Karl Fischer (KF) titration on form output using the above described drying process is summarized in FIG. 2. The effect of water content in the reslurry solvent on observed form is summarized in FIG. 3.

TABLE 3

| | | | Form Transformation |
|---|---|---|---|
| Reslurry | # | KF wet | Observed Upon Drying Raman |
| Composition | reslurries | cake % | (Final state XRPD verified) |
| 100% EtOH | 4 | 4.97 | C to A |
| 100% EtOH | 2 | 8.88 | C to A |
| 95% EtOH | 2 | 13.05 | C to A |
| 93% EtOH | 2 | 16.74 | C to E/A |
| 90% EtOH | 2 | 17.5 | D |
| 80% EtOH | 2 | 18.74 | D |
| 60% EtOH | 2 | 35.12 | D |
| 0% EtOH | 2 | — | gel |

The effect of filtration temperature and vacuum drying procedure on observed form was also assessed, and the results are summarized in TABLE 4 and TABLE 5.

TABLE 4

| Filtration | Raman Characterisation | | |
|---|---|---|---|
| Temperature | Pre-Drying | Post 25° C. drying | Post 50° C. drying |
| 10° C. | C | A | A |
| 10° C. | C | n/a | A (XCRPD) |
| 15° C. | C | A | A |
| 20° C. | n/a | n/a | B |
| 30° C. | D | n/a | * (XRPD) |

* XRPD pattern did not match known standards.
n/a: step not performed

TABLE 5

| Reslurry | | Transformation | |
|---|---|---|---|
| solvent | Drying | Observed | Form |
| composition | Parameter | (Raman) | Output |
| | Vacuum | — | — |
| 2 × 100% EtOH | 4 mbar for 4 h at | C to A | A (XRPD) |
| | 25° C., then 50° C. | | |
| | at 4 mbar for 12 h | | |
| 2 × 100% EtOH | 4 mbar for 4 h at | C to A | A (XRPD) |
| | 25° C., then 50° C. | | |
| | at 4 mbar for 12 h | | |
| 2 × 100% EtOH | 90 mbar for 4 h at | C to A | A (XRPD) |
| | 25° C., then 50° C. | | |
| | at 90 mbar for 12 h | | |
| 2 × 95% EtOH | 0 mbar for 4 h at | C to A | A (XRPD) |
| (KF~13 w/w %) | 25° C., then 50° C. | | |
| | at 0 mbar for 44 h | | |
| 2 × 95% EtOH | 90 mbar for 4 h at | C to D to B | B (XRPD) |
| (KF~14 w/w %) | 25° C., then 50° C. | | |
| | at 90 mbar over | | |
| | the weekend; then | | |
| | 40 mbar for 4 h at | | |
| | 25° C. followed by | | |
| | 50° C. overnight | | |
| | (~16 h ) | | |
| No washes | 90 mbar for 4 h at | C to D to B | B (XRPD) |
| | 25° C., then 90 | | |
| | mbar at 50° C. | | |
| | overnight | | |

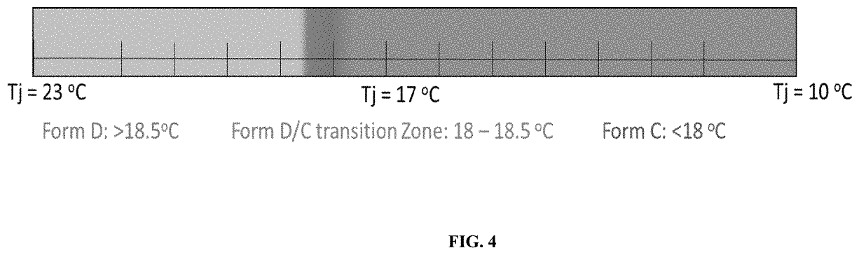

The effect of reslurry solvent temperature on observed form was also assessed. Compound 10 was suspended in ethanol containing 2.9 w/w % water, and starting from 10° C., the temperature was raised stepwise 1° C. per minute, followed by a hold time of 30 minutes. Compound form was monitored via Raman spectroscopy. The results are summarized in FIG. 4. Form C was observed below 18° C. A form C to D transition was observed between 18° C. and 18.5° C. Form D was observed above 18.5° C.

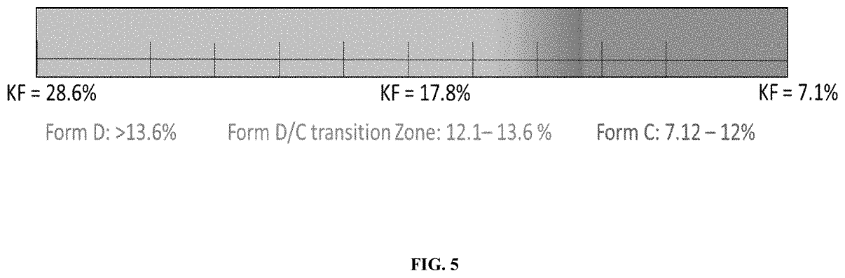

In a separate study, guadecitabine was suspended in ethanol containing 7 w/w % water, and 0.1 mL water was added every 30 minutes followed by a hold time of 30 minutes. Compound form was monitored via Raman spectroscopy. The temperature of the suspension was maintained at 10° C. throughout the study. The results are summarized in FIG. 5. Form C was observed below 12 w/w % water. A form C to D transition was observed between 12.1 and 13.6 w/w % water. Form D was observed above 13.6 w/w % water.

### Example 7: Characterization of Compound 10 Solid Forms Via X-Ray Powder Diffraction

Forms A, B, C, and D were characterized by X-ray powder diffraction (XRPD). XRPD diffractograms were collected with a PANalytical X'Pert PRO MPD® diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. For the determination of solid forms under the environmental conditions surveyed in EXAMPLE 6, samples of Compound 10 were stored within environmental chambers maintained at specific relative humidity conditions. A specimen of the sample was sandwiched between 3-μm-thick Etnom® films within these environmental chambers. Once prepared, the test article was removed from the environmental chamber and analyzed in transmission geometry without undue delay. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b.

### Form A.

Observed peaks for Form A are shown in FIG. 6 and summarized in TABLE 6.

TABLE 6

| °2θ (±0.20) | d space (Å) | Intensity (%) |
|---|---|---|
| 5.64 | 15.667 ± 0.555 | 24 |
| 10.10 | 8.748 ± 0.173 | 49 |
| 11.18 | 7.911 ± 0.141 | 100 |
| 13.98 | 6.330 ± 0.090 | 30 |
| 14.89 | 5.946 ± 0.079 | 71 |
| 15.30 | 5.788 ± 0.075 | 40 |
| 17.73 | 4.997 ± 0.056 | 29 |
| 18.41 | 4.816 ± 0.052 | 20 |
| 19.32 | 4.589 ± 0.047 | 31 |
| 19.68 | 4.508 ± 0.045 | 24 |
| 20.37 | 4.357 ± 0.042 | 25 |
| 21.45 | 4.139 ± 0.038 | 21 |
| 21.75 | 4.083 ± 0.037 | 22 |
| 22.53 | 3.944 ± 0.035 | 23 |
| 23.89 | 3.721 ± 0.031 | 56 |
| 24.86 | 3.579 ± 0.028 | 22 |
| 25.86 | 3.442 ± 0.026 | 21 |
| 26.45 | 3.367 ± 0.025 | 14 |
| 27.41 | 3.251 ± 0.023 | 19 |

### Form B.

Observed peaks for Form B are shown in FIG. 7 and summarized in TABLE 7.

TABLE 7

| °2θ (±0.20) | d space (Å) | Intensity (%) |
|---|---|---|
| 2.54 | 34.7 ± 2.727 | 11 |
| 5.13 | 17.207 ± 0.670 | 72 |
| 5.39 | 16.368 ± 0.606 | 8 |
| 6.34 | 13.929 ± 0.439 | 12 |
| 6.77 | 13.04 ± 0.385 | 9 |
| 8.08 | 10.939 ± 0.270 | 13 |
| 10.23 | 8.639 ± 0.168 | 100 |
| 10.79 | 8.196 ± 0.152 | 9 |
| 11.17 | 7.915 ± 0.141 | 40 |
| 11.42 | 7.742 ± 0.135 | 16 |
| 11.65 | 7.589 ± 0.130 | 16 |
| 11.89 | 7.434 ± 0.125 | 10 |
| 12.15 | 7.28 ± 0.119 | 7 |
| 12.57 | 7.036 ± 0.111 | 37 |
| 13.25 | 6.676 ± 0.100 | 6 |
| 14.33 | 6.177 ± 0.086 | 18 |
| 14.77 | 5.992 ± 0.081 | 9 |
| 15.48 | 5.718 ± 0.073 | 15 |
| 16.00 | 5.534 ± 0.069 | 12 |
| 16.34 | 5.422 ± 0.066 | 11 |
| 17.30 | 5.122 ± 0.059 | 8 |
| 18.44 | 4.807 ± 0.052 | 13 |
| 18.81 | 4.713 ± 0.050 | 20 |
| 19.25 | 4.608 ± 0.047 | 21 |
| 19.89 | 4.461 ± 0.044 | 14 |
| 20.05 | 4.425 ± 0.044 | 12 |
| 20.55 | 4.318 ± 0.042 | 25 |
| 21.43 | 4.144 ± 0.038 | 14 |
| 21.78 | 4.077 ± 0.037 | 27 |
| 22.34 | 3.976 ± 0.035 | 56 |
| 23.20 | 3.831 ± 0.033 | 35 |
| 23.92 | 3.718 ± 0.031 | 15 |
| 24.21 | 3.674 ± 0.030 | 15 |
| 25.43 | 3.499 ± 0.027 | 8 |
| 26.86 | 3.316 ± 0.024 | 14 |
| 28.40 | 3.14 ± 0.022 | 8 |
| 29.01 | 3.076 ± 0.021 | 7 |

### Form C.

Observed peaks for Form C are shown in FIG. 8 and summarized in TABLE 8.

TABLE 8

| °2θ (±0.20) | d space (Å) | Intensity (%) |
|---|---|---|
| 9.34 | 55 | 9.468812 |
| 10.04 | 20 | 8.810153 |
| 10.84 | 83 | 8.161688 |
| 11.73 | 21 | 7.544354 |
| 12.03 | 37 | 7.356882 |
| 12.28 | 30 | 7.207665 |
| 13.25 | 26 | 6.682111 |
| 13.85 | 29 | 6.393954 |
| 14.17 | 19 | 6.250272 |
| 15.02 | 17 | 5.89842 |
| 15.35 | 28 | 5.772349 |
| 16.44 | 25 | 5.392006 |
| 17.73 | 19 | 5.002497 |
| 18.75 | 28 | 4.732599 |
| 19.25 | 26 | 4.610788 |
| 19.35 | 29 | 4.587185 |
| 19.65 | 34 | 4.517824 |
| 19.97 | 49 | 4.446147 |
| 20.47 | 52 | 4.338661 |
| 22.39 | 75 | 3.970765 |
| 22.51 | 100 | 3.949868 |
| 24.78 | 36 | 3.592942 |
| 26.12 | 61 | 3.411579 |
| 29.21 | 20 | 3.057336 |
| 29.98 | 25 | 2.980544 |
| 30.96 | 17 | 2.888397 |
| 33.03 | 18 | 2.71196 |

### Form D.

Observed peaks for Form D are shown in FIG. 9 and summarized in TABLE 9. Multiple values in TABLE 9 are representative of differing measurements from two separate scans.

TABLE 9

| °2θ (±0.20) | d space (Å) | Intensity (%) |
|---|---|---|
| 4.90 | 18.007 ± 0.734 | 13, 86 |
| 5.10, 5.22 | 17.321 ± 0.679, 16.929 ± 0.649 | 0, 11 |
| 7.09, 7.15 | 12.450 ± 0.351, 12.347 ± 0.345 | 8, 9 |
| 9.96, 10.01 | 8.871 ± 0.178, 8.833 ± 0.176 | 66, 74 |
| 10.25, 10.4 | 8.626 ± 0.168-8.484 ± 0.162 | 0, 4 |
| 11.24, 11.25 | 7.865 ± 0.139, 7.859 ± 0.139 | 43, 54 |
| 11.36, 11.52 | 7.785 ± 0.137, 7.677 ± 0.133 | 18, 20 |
| 12.16, 12.20 | 7.273 ± 0.119, 7.248 ± 0.118 | 30, 56 |
| 13.23, 13.24 | 6.684 ± 0.101, 6.682 ± 0.101 | 11, 23 |
| 14.33, 14.44 | 6.175 ± 0.086, 6.128 ± 0.084 | 6, 12 |
| 14.77, 14.81 | 5.991 ± 0.081, 5.977 ± 0.080 | 9, 16 |
| 15.67, 15.69 | 5.650 ± 0.072, 5.644 ± 0.072) | 8, 10 |
| 15.95, 16.04 | 5.551 ± 0.069, 5.520 ± 0.068 | 5, 12 |
| 16.52, 16.54 | 5.362 ± 0.064, 5.356 ± 0.064 | 14, 19 |
| 18.04, 18.11 | 4.912 ± 0.054, 4.893 ± 0.054 | 4, 9 |
| 18.39, 18.61 | 4.820 ± 0.052, 4.765 ± 0.051 | 20, 24 |
| 18.80, 18.85 | 4.716 ± 0.050, 4.703 ± 0.049 | 7, 12 |
| 19.40, 19.49 | 4.571 ± 0.047, 4.551 ± 0.046 | 4, 11 |
| 19.68, 19.88 | 4.506 ± 0.045, 4.461 ± 0.044 | 10, 26 |
| 20.03, 20.11 | 4.42 ± 0.044, 4.412 ± 0.043 | 39, 54 |
| 20.56, 20.95 | 4.316 ± 0.042, 4.238 ± 0.040 | 100 |

### Example 8: Analytical Methods for Purity Assessment

The purity of Compound 9 or Compound 10 can be assessed according to the method summarized in TABLE 10. Selected impurities observable via the disclosed method are summarized in TABLE 11.

TABLE 10

| Column | C18 150 × 4.6 mm, 5 μm | | |
|---|---|---|---|
| Mobile phase | A: 2.9% MeCN in 10 mM phosphate buffer pH 6.5 | | |
| | B: MeCN | | |
| Gradient | Time (min) | A(%) | B(%) |
| | 0.0 | 100 | 0 |
| | 9.0 | 100 | 0 |
| | 22.0 | 93 | 7 |
| | 30.0 | 27 | 73 |
| | 33.0 | 27 | 73 |
| | 35.0 | 100 | 0 |
| | 40.0 | 100 | 0 |
| Flow rate: | 1.2 mL/min | | |
| Column | 25° C. | | |
| Temperature | | | |
| Wavelength | 250 nm | | |
| Injection Volume | 2 μL | | |
| Wash vial | MeCN/H₂O (1:1) | | |
| Sample Diluent | 3 × DMSO:MeOH (1:1) | | |

Compound 10 and Compound 12 have retention times of approximately 13.6 minutes (RRT=1) and 19.4 minutes (RRT=1.42), respectively, when the HPLC method of TABLE 10 is used.

### EMBODIMENTS

Embodiment A1. A compound of formula (IVa):

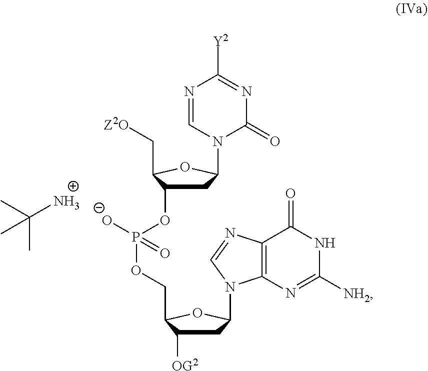

wherein:
each Z² and G² is independently H or a protecting group; and
each Y² and Q² is independently NH₂ or a protected primary amine.

Embodiment A2. The compound of embodiment A1, wherein each Z² and G² is independently H, substituted or unsubstituted acetyl, substituted or unsubstituted phenoxyacetyl, substituted or unsubstituted ethoxymethyl, substituted or unsubstituted benzoyl, or a silyl protecting group.

Embodiment A3. The compound of embodiment A1, wherein each Z² and G² is independently H, benzoyl, acetyl, isobutyryl, Pac, Tac, iPr-Pac, [(triisopropylsilyl)oxy]methyl, tert-butyldimethylsilyl, or 2′-cyanoethoxymethyl.

Embodiment A4. The compound of embodiment A1, wherein each Z² and G² is independently H, acetyl, Pac, Tac, or iPr-Pac.

Embodiment A5. The compound of embodiment A1, wherein Z² is H or acetyl.

Embodiment A6. The compound of embodiment A1 or embodiment A5, wherein G² is H or Tac.

Embodiment A7. The compound of any one of embodiments A1-A6, wherein each Y² and Q² is independently NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or a formamidine group.

Embodiment A8. The compound of any one of embodiments A1-A6, wherein each Y² and Q² is independently NH₂, or a primary amine protected with benzoyl, acetyl, isobutyryl, Pac, Tac, iPr-Pac, [(triisopropylsilyl)oxy]methyl, tert-butyldimethylsilyl, or 2′-cyanoethoxymethyl.

Embodiment A9. The compound of any one of embodiments A1-A6, wherein each Y² and Q² is independently NH₂, or a primary amine protected with acetyl, Pac, Tac, iPr-Pac.

Embodiment A10. The compound of any one of embodiments A1-A6, wherein Y² is NH₂ or NH(Tac).

Embodiment A11. The compound of any one of embodiments A1-A6 and A10, wherein Q² is NH₂ or NH(Tac).

Embodiment A12. The compound of embodiment A1, wherein the compound is:

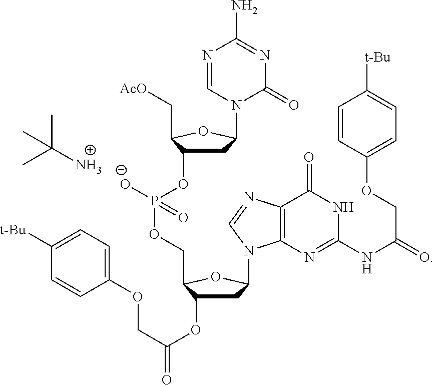

Embodiment B1. A process comprising contacting a solution with a base, wherein the solution comprises a compound of formula (III):

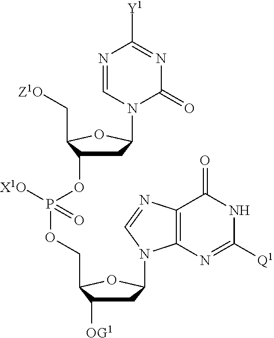

to provide an ion pair of formula (IVb):

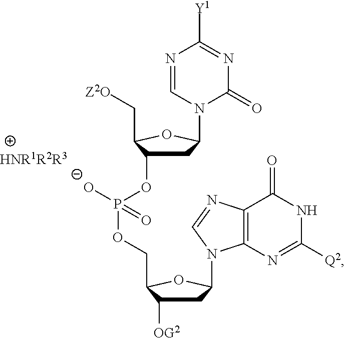

wherein:
the base is NR¹R²R³;
X¹ is 2-cyanoethyl, 2,2,2-trichloroethyl, 2,2,2-tribromoethyl, 2,2,2-trifluoroethyl, benzyl, p-chloroethyl, or p-nitroethyl;
R′, R², and R³ are each independently H or branched or unbranched alkyl, wherein at least one of R′, R², and R³ is not H; or R¹ is H or branched or unbranched alkyl and R² and R³ taken together with the atom to which R² and R³ are bound form a ring;
each Z¹, Z², and G² is independently H or a protecting group;
Y¹ is a protected primary amine; and
each Y², Q¹, and Q² is independently NH₂ or a protected primary amine.

Embodiment B2. The process of embodiment B1, wherein the solution further comprises acetonitrile.

Embodiment B3. The process of embodiment B1, wherein the solution further comprises acetonitrile and tetrahydrofuran.

Embodiment B4. The process of any one of embodiments B1-B3, wherein the solution comprises no more than 1% (w/w) dichloromethane.

Embodiment B5. The process of any one of embodiments B1-B4, wherein the base is tert-butylamine.

Embodiment B6. The process of any one of embodiments B1-B5, wherein each Z¹ and G¹ is independently H, substituted or unsubstituted acetyl, substituted or unsubstituted phenoxyacetyl, substituted or unsubstituted ethoxymethyl, substituted or unsubstituted benzoyl, or a silyl protecting group.

Embodiment B7. The process of any one of embodiments B1-B5, wherein each Z¹ and G¹ is independently H, benzoyl, acetyl, isobutyryl, Pac, Tac, iPr-Pac, [(triisopropylsilyl)oxy]methyl, tert-butyldimethylsilyl, or 2′-cyanoethoxymethyl.

Embodiment B8. The process of any one of embodiments B1-B5, wherein each Z¹ and G¹ is independently H, acetyl, Pac, Tac, or iPr-Pac.

Embodiment B9. The process of any one of embodiments B1-B5, wherein Z¹ is H or acetyl.

Embodiment B10. The process of any one of embodiments B1-B5 and B9, wherein G¹ is H or Tac.

Embodiment B11. The process of any one of embodiments B1-B10, wherein Q¹ is NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or a formamidine group.

Embodiment B12. The process of any one of embodiments B1-B10, wherein Q¹ is NH₂, or a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl.

Embodiment B13. The process of any one of embodiments B1-B10, wherein Q¹ is NH₂, or a primary amine protected with ═CHN(CH₃)₂, acetyl, Pac, Tac, or iPr-Pac.

Embodiment B14. The process of any one of embodiments B1-B10, wherein Q¹ is NH₂ or NH(Tac).

Embodiment B15. The process of any one of embodiments B1-B14, wherein Y¹ is a primary amine protected with substituted or unsubstituted acetyl, substituted or unsubstituted phenoxyacetyl, substituted or unsubstituted ethoxymethyl, substituted or unsubstituted benzoyl, a silyl protecting group, or a formamidine group.

Embodiment B16. The process of any one of embodiments B1-B14, wherein Y¹ is a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl.

Embodiment B17. The process of any one of embodiments B1-B14, wherein Y¹ is a primary amine protected with ═CHN(CH₃)₂, acetyl, Pac, Tac, or iPr-Pac.

Embodiment B18. The process of any one of embodiments B1-B14, wherein Y¹ is N═CHN(CH₃)₂ or NH(Tac)

Embodiment B19. The process of any one of embodiments B1-B18, wherein each Z² and G² is independently H, substituted or unsubstituted acetyl, substituted or unsubstituted phenoxyacetyl, substituted or unsubstituted ethoxymethyl, substituted or unsubstituted benzoyl, or a silyl protecting group.

Embodiment B20. The process of any one of embodiments B1-B18, wherein each Z² and G² is independently H, benzoyl, acetyl, isobutyryl, Pac, Tac, iPr-Pac, [(triisopropylsilyl)oxy]methyl, tert-butyldimethylsilyl, or 2′-cyanoethoxymethyl.

Embodiment B21. The process of any one of embodiments B1-B18, wherein each Z² and G² is independently H, acetyl, Pac, Tac, or iPr-Pac.

Embodiment B22. The process of any one of embodiments B1-B18, wherein Z² is H or acetyl.

Embodiment B23. The process of any one of embodiments B1-B18 and B22, wherein G² is H or Tac.

Embodiment B24. The process of any one of embodiments B1-B23, wherein each Y² and Q² is independently NH₂, or a primary amine protected with substituted or unsubstituted acetyl, substituted or unsubstituted phenoxyacetyl, substituted or unsubstituted ethoxymethyl, substituted or unsubstituted benzoyl, or a silyl protecting group.

Embodiment B25. The process of any one of embodiments B1-B23, wherein each Y² and Q² is independently NH₂, or a primary amine protected with benzoyl, acetyl, isobutyryl, Pac, Tac, iPr-Pac, [(triisopropylsilyl)oxy]methyl, tert-butyldimethylsilyl, or 2′-cyanoethoxymethyl.

Embodiment B26. The process of any one of embodiments B1-B23, wherein each Y² and Q² is independently NH₂, or a primary amine protected with acetyl, Pac, Tac, or iPr-Pac.

Embodiment B27. The process of any one of embodiments B1-B23, wherein Y² is NH₂ or NH(Tac).

Embodiment B28. The process of any one of embodiments B1-B23 and B27, wherein Q² is NH₂ or NH(Tac).

Embodiment B29. The process of any one of embodiments B1-B28, wherein X¹ is 2-cyanoethyl.

Embodiment B30. The process of any one of embodiments B1-B29, wherein R¹ and R² are each H, and R³ is tert-butyl.

Embodiment B31. The process of any one of embodiments B1-B5, wherein the compound is

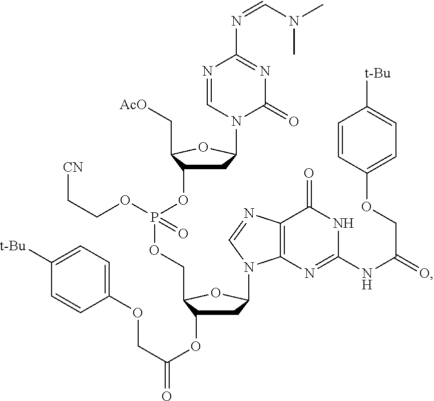

and
the ion pair is:

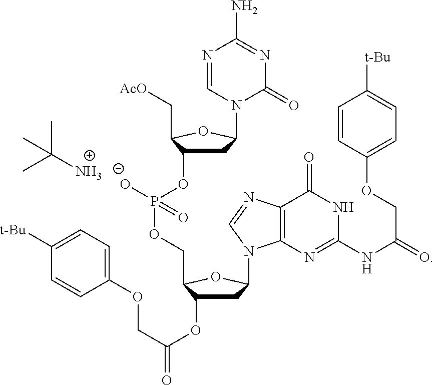

Embodiment B32. The process of any one of embodiments B1-B5, wherein Y¹ and Q¹ are each independently N═CN(CH₃)₂, NHR^{A}, NHR^{B}, or NH₂, wherein:

if Y¹ is N═C(CH₃)₂ or NH₂, then Y² is NH₂;

if Q¹ is N═C(CH₃)₂ or NH₂, then Q² is NH₂;

if Y¹ is NHR^{A}, then Y² is NHR^{A};

if Y¹ is NHR^{B}, then Y² is NHR^{B};

if is NHR^{A}, then Q² is NHR^{A};

if Q¹ is NHR^{B}, then Q² is NHR^{B}; and
Z¹ and G¹ are each independently H, R^{C}, or R^{D}, wherein:

if Z¹ is H, then Z² is H;

if G¹ is H, then G² is H;

if Z¹ is R^{C}, then Z² is R^{C};

if Z¹ is R^{D}, then Z² is R^{D};

if G¹ is R^{C}, then G² is R^{C};

if G¹ is R^{D}, then G² is R^{D}; and
each R^{A}, R^{B}, R^{C}, and R^{D} is acetyl, Pac, Tac, or iPr-Pac.

Embodiment C1. A process comprising:
(i) contacting a solution with a base, wherein the solution comprises a first compound of formula (IV):

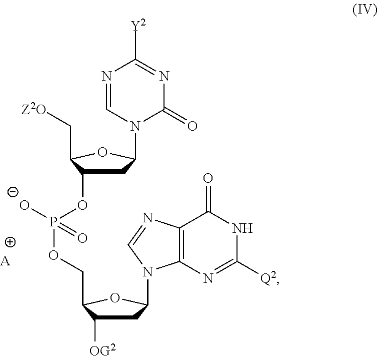

to provide a reaction mixture; and
(ii) contacting the reaction mixture with an acid to provide a second reaction mixture, wherein the second reaction mixture comprises a second compound, wherein the second compound is:

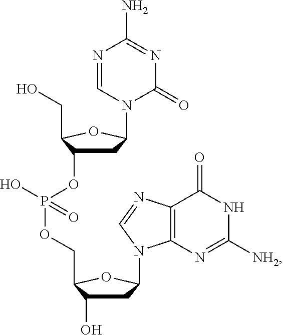

wherein:
A⁺ is an alkylammonium cation;
Z² and G² are each independently H, substituted or unsubstituted acetyl, or substituted or unsubstituted phenoxyacetyl; and
Y² and Q² are each independently NH₂, or a primary amine protected with substituted or unsubstituted acetyl, or substituted or unsubstituted phenoxyacetyl.

Embodiment C2. The process of embodiment C1, wherein A⁺ is ⁺HNR¹R²R³, wherein R¹, R², and R³ are each independently H or branched or unbranched alkyl, wherein at least one of R¹, R², and R³ is not H; or R¹ is H or branched or unbranched alkyl and R² and R³ are taken together with the atom to which R² and R³ are bound to form a ring.

Embodiment C3. The process of embodiment C1 or embodiment C2, wherein A⁺ is a tert-butylammonium cation.

Embodiment C4. The process of any one of embodiments C1-C3, wherein Z² and G² are each independently substituted or unsubstituted acetyl, or substituted or unsubstituted phenoxyacetyl.

Embodiment C5. The process of any one of embodiments C1-C3, wherein Z² is substituted or unsubstituted acetyl, and G² is substituted or unsubstituted phenoxyacetyl.

Embodiment C6. The process of any one of embodiments C1-C3, wherein Z² and G² are each independently H, acetyl, Pac, Tac, or iPr-Pac.

Embodiment C7. The process of any one of embodiments C1-C3, wherein Z² and G² are each independently acetyl, Pac, Tac, or iPr-Pac.

Embodiment C8. The process of any one of embodiments C1-C3, wherein G² is Pac, Tac, or iPr-Pac.

Embodiment C9. The process of any one of embodiments C1-C3 and C8, wherein Z² is acetyl.

Embodiment C10. The process of any one of embodiments C1-C3, wherein G² is Pac.

Embodiment C11. The process of any one of embodiments C1-C10, wherein Q² is a primary amine protected with substituted or unsubstituted acetyl, or a primary amine protected with substituted or unsubstituted phenoxyacetyl.

Embodiment C12. The process of any one of embodiments C1-C10, wherein Q² is a primary amine protected with substituted or unsubstituted phenoxyacetyl.

Embodiment C13. The process of any one of embodiments C1-C10, wherein Q² is NHAc, NHPac, iPrNHPac, or NHTac.

Embodiment C14. The process of any one of embodiments C1-C13, wherein Y² is NH₂.

Embodiment C15. The process of any one of embodiments C1-C10, wherein Y² and Q² are each independently acetyl, NHAc, NHPac, iPrNHPac, NHTac, or NH₂.

Embodiment C16. The process of embodiment C1, wherein the first compound is:

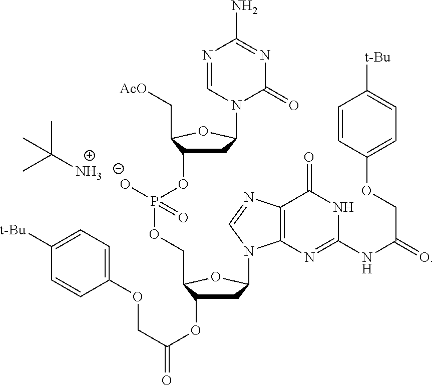

Embodiment C17. The process of any one of embodiments C1-C16, wherein the solution further comprises a C₁-C₆ alcohol.

Embodiment C18. The process of any one of embodiments C1-C16, wherein the solution further comprises methanol.

Embodiment C19. The process of embodiment C18, further comprising removing methanol from the second reaction mixture via distillation to provide a concentrate.

Embodiment C20. The process of embodiment C19, further comprising adding DMSO to the concentrate to provide a second solution.

Embodiment C21. The process of embodiment C19, further comprising adding DMSO and methanol to the concentrate to provide a second solution.

Embodiment C22. The process of embodiment C20 or embodiment C21, further comprising filtering the second solution to provide a filtrate, and cooling the filtrate to a temperature no more than about 20° C.

Embodiment C23. The process of embodiment C22, further comprising contacting a sodium cation source with the filtrate to provide a third solution, wherein the third solution comprises an ion pair, wherein the ion pair is:

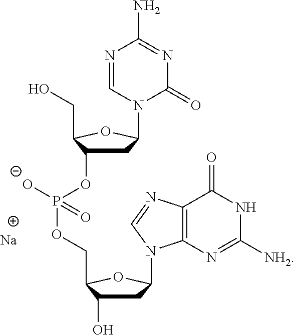

Embodiment C24. The process of embodiment C23, wherein the sodium cation source is a sodium carboxylate.

Embodiment C25. The process of embodiment C23, wherein the sodium cation source is sodium acetate.

Embodiment C26. The process of any one of embodiments C23-C25, further comprising contacting the third solution with an antisolvent with respect to the ion pair to provide a mixture comprising a solid, the solid comprising the ion pair.

Embodiment C27. The process of embodiment C26, wherein the antisolvent is ethanol.

Embodiment C28. The process of embodiment C26 or embodiment C27, further comprising filtering the mixture to provide a retentate, wherein the retentate comprises the ion pair.

Embodiment C29. The process of embodiment C28, further comprising washing the retentate with ethanol.

Embodiment C30. The process of any one of embodiments C1-C29, wherein the base is an alkoxide.

Embodiment C31. The process of any one of embodiments C1-C29, wherein the base is a sodium alkoxide.

Embodiment C32. The process of any one of embodiments C1-C29, wherein the base is sodium methoxide.

Embodiment C33. The process of any one of embodiments C1-C32, wherein the acid is a carboxylic acid.

Embodiment C34. The process of any one of embodiments C1-C32, wherein the acid is acetic acid.

Embodiment C35. The process of any one of embodiments C1-C34, wherein the reaction mixture comprises no more than 0.3% (a/a) of a third compound as determined by HPLC, wherein the third compound is:

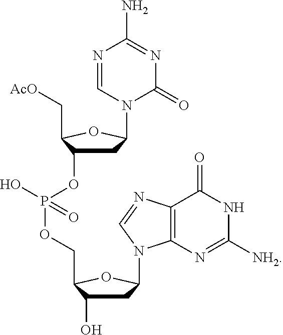

Embodiment C36. The process of any one of embodiments C1-C35, wherein the second compound is at least 99.5% (a/a) of the reaction mixture as determined by HPLC.

Embodiment D1. A process comprising contacting a first solution with a lipase and an acetyl donor to provide a second solution, wherein the first solution comprises a compound of formula (Ia):

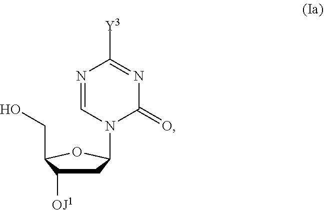

wherein the second solution comprises a compound of formula (Id):

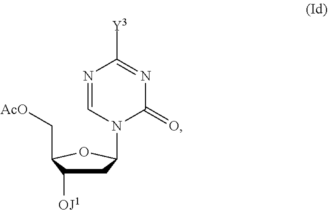

wherein:
the lipase is Novozym® 40086;
Y³ is NH₂ or a protected primary amine; and
J¹ is H, or a protecting group.

Embodiment D2. The process of embodiment D1, wherein Y³ is NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or a formamidine group.

Embodiment D3. The process of embodiment D1 or embodiment D2, wherein J¹ is H, substituted or unsubstituted acetyl, substituted or unsubstituted phenoxyacetyl, substituted or unsubstituted ethoxymethyl, substituted or unsubstituted benzoyl, or a silyl protecting group.

Embodiment D4. The process of any one of embodiments D1-D3, wherein Y³ is NH₂, or a primary amine protected with ═CHN(CH₃)₂, benzoyl, acetyl, isobutyryl, Pac, Tac, iPr-Pac, [(triisopropylsilyl)oxy]methyl, tert-butyldimethylsilyl, or 2′-cyanoethoxymethyl.

Embodiment D5. The process of any one of embodiments D1-D4, wherein J¹ is H, benzoyl, acetyl, isobutyryl, Pac, Tac, iPr-Pac, [(triisopropylsilyl)oxy]methyl, tert-butyldimethylsilyl, or 2′-cyanoethoxymethyl,

Embodiment D6. The process of any one of embodiments D1-D5, wherein Y³ is NH₂, or a primary amine protected with ═CHN(CH₃)₂, acetyl, Pac, Tac, iPr-Pac.

Embodiment D7. The process of any one of embodiments D1-D6, wherein J¹ is H, acetyl, Pac, Tac, or iPr-Pac.

Embodiment D8. The process of any one of embodiments D1-D7, wherein Y³ is N═CHN(CH₃)₂.

Embodiment D9. The process of any one of embodiments D1-D8, wherein J¹ is H.

Embodiment D10. The process of any one of embodiments D1-D10, wherein the acetyl donor is vinyl acetate, isopropenyl acetate, ethyl acetate, or acetic anhydride.

Embodiment D11. The process of any one of embodiments D1-D10, wherein the acetyl donor is vinyl acetate.

Embodiment D12. The process of any one of embodiments D1-D11, wherein the first solution further comprises an organic solvent.

Embodiment D13. The process of any one of embodiments D1-D11, wherein the first solution further comprises an organic solvent, and the organic solvent comprises 1,4-dioxane.

Embodiment D14. The process of any one of embodiments D1-D13, wherein the first solution further comprises an organic solvent, and the organic solvent comprises acetonitrile.

Embodiment D15. The process of any one of embodiments D1-D11, wherein the first solution further comprises an organic solvent, and the organic solvent comprises 1,4-dioxane and acetonitrile.

Embodiment D16. The process of any one of embodiments D1-D15, further comprising filtering the second solution to provide a filtrate.

Embodiment D17. The process of embodiment D16, further comprising contacting the filtrate with an aliphatic solvent to provide a suspension, and filtering the suspension to provide a retentate.

Embodiment D18. The process of embodiment D17, wherein the aliphatic solvent is n-heptane.

Embodiment D19. The process of embodiment D17 or embodiment D18, further comprising contacting the retentate with a halogenated solvent to provide a third solution.

Embodiment D20. The process of embodiment D19, further comprising contacting the third solution with a mixture of acetone and at least one seed crystal of the compound of formula (Id).

Embodiment D21. The process of embodiment D19 or embodiment D20, wherein the halogenated solvent is dichloromethane.

Embodiment E1. A process for producing a polymorph of Compound 10:

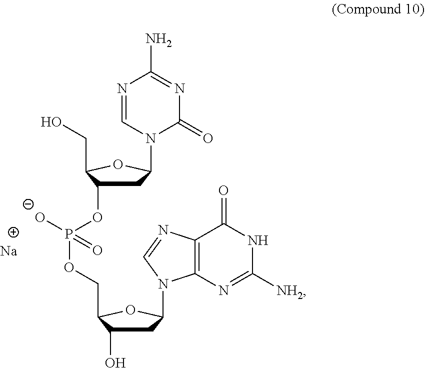

comprising:
(i) mixing Compound 10 with ethanol to provide a suspension;
(ii) filtering the suspension to provide a retentate; and
(iii) drying the retentate under reduced pressure to provide the polymorph, wherein:
the ethanol has a water content that is no more than 7% (w/w).

Embodiment E2. The process of embodiment E1, wherein the reduced pressure is no more than 200 mbar.

Embodiment E3. The process of embodiment E1, wherein the reduced pressure is no more than 150 mbar.

Embodiment E4. The process of embodiment E1, wherein the reduced pressure is no more than 100 mbar.

Embodiment E5. The process of embodiment E1, wherein the reduced pressure is from about 0.1 mbar to about 100 mbar.

Embodiment E6. The process of any one of embodiments E1-E5, wherein the drying under reduced pressure further comprises heating at a temperature of at least 40° C.

Embodiment E7. The process of any one of embodiments E1-E5, wherein the drying under reduced pressure further comprises heating at a temperature from about 40° C. to about 80° C.

Embodiment E8. The process of any one of embodiments E1-E7, wherein the retentate has an X-ray powder diffraction pattern that comprises peaks at 4.9°, 7.2°, and 10.0°±0.2 2θ as measured by X-ray powder diffraction using Cu K alpha radiation.

Embodiment E9. The process of embodiment E8, wherein the X-ray powder diffraction pattern further comprises peaks at 11.3°, 11.5°, and 12.2°±0.2 2θ as measured by X-ray powder diffraction using Cu K alpha radiation.

Embodiment E10. The process of any one of embodiments E1-E9, wherein the polymorph has an X-ray powder diffraction pattern that comprises peaks at 10.1°, 11.2°, and 14°±0.2 2θ as measured by X-ray powder diffraction using Cu K alpha radiation.

Embodiment E11. The process of embodiment E10, wherein the X-ray powder diffraction pattern of the polymorph further comprises peaks at 15.3°, 17.7°, and 18.4°±0.2 2θ as measured by X-ray powder diffraction using Cu K alpha radiation.

Embodiment F1. A process for producing a polymorph of Compound 10:

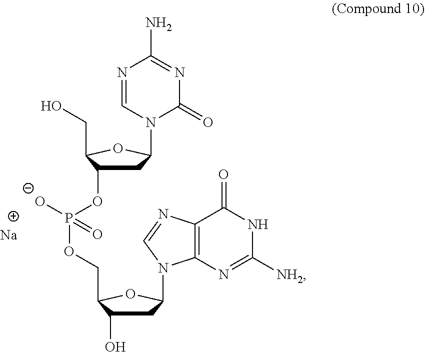

comprising drying under reduced pressure a solid form of Compound 10 to provide the polymorph, wherein the solid form of Compound 10 has an X-ray powder diffraction pattern that comprises peaks at 4.9°, 7.2°, and 10.0°±0.2 2θ as measured by X-ray powder diffraction using Cu K alpha radiation.

Embodiment F2. The process of embodiment F1, wherein the X-ray powder diffraction pattern of the solid form further comprises peaks at 11.3°, 11.5°, and 12.2°±0.2 2θ as measured by X-ray powder diffraction using Cu K alpha radiation.

Embodiment F3. The process of embodiment F1 or embodiment F2, wherein the polymorph has an X-ray powder diffraction pattern that comprises peaks at 10.1°, 11.2°, and 14°±0.2 2θ as measured by X-ray powder diffraction using Cu K alpha radiation.

Embodiment F4. The process of embodiment F3, wherein the X-ray powder diffraction pattern of the polymorph further comprises peaks at 15.3°, 17.7°, and 18.4°±0.2 2θ as measured by X-ray powder diffraction using Cu K alpha radiation.

Embodiment F5. The process of any one of embodiments F1-F4, wherein the reduced pressure is no more than 200 mbar.

Embodiment F6. The process of any one of embodiments F1-F4, wherein the reduced pressure is no more than 150 mbar.

Embodiment F7. The process of any one of embodiments F1-F4, wherein the reduced pressure is no more than 100 mbar.

Embodiment F8. The process of any one of embodiments F1-F4, wherein the reduced pressure is from about 0.1 mbar to about 100 mbar.

Embodiment F9. The process of any one of embodiments F1-F8, wherein the drying under reduced pressure further comprises heating at a temperature of at least 40° C.

Embodiment F10. The process of any one of embodiments F1-F8, wherein the drying under reduced pressure further comprises heating at a temperature from about 40° C. to about 80° C.

Embodiment G1. A composition comprising a solid form of Compound 10:

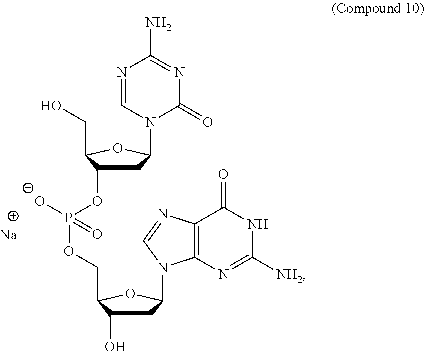

wherein the solid form exhibits an X-ray powder diffraction pattern substantially the same as the X-ray powder diffraction pattern shown in FIG. 6.

Embodiment H1. A composition comprising a solid form of Compound 10:

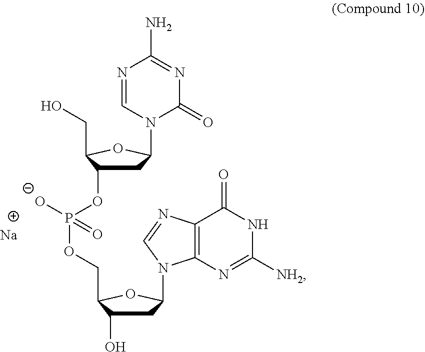

wherein the solid form has an X-ray powder diffraction pattern that comprises peaks at 10.1°, 11.2°, and 14°±0.2 2θ as measured by X-ray powder diffraction using Cu K alpha radiation.

Embodiment H2. The composition of embodiment H1, wherein the X-ray powder diffraction pattern further comprises peaks at 15.3°, 17.7°, and 18.4°±0.2 2θ as measured by X-ray powder diffraction using Cu K alpha radiation.

Embodiment I1. A composition comprising a solid form of Compound 10:

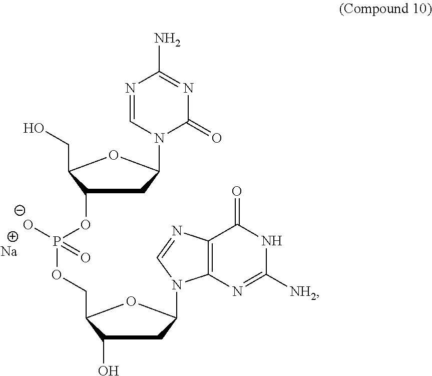 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

wherein the solid form exhibits an X-ray powder diffraction pattern substantially the same as the X-ray powder diffraction pattern shown in FIG. 7.

Embodiment J1. A composition comprising a solid form of Compound 10:

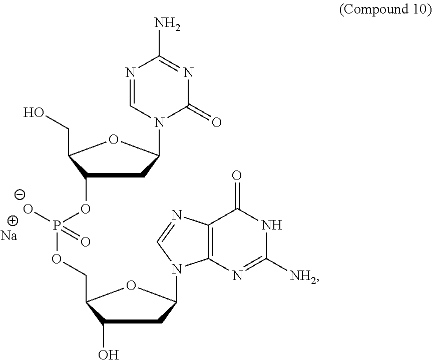 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

wherein the solid form has an X-ray powder diffraction pattern that comprises peaks at 5.1°, 10.2°, and 11.2°±0.2 2θ as measured by X-ray powder diffraction using Cu K alpha radiation.

Embodiment J2. The composition of embodiment J1, wherein the X-ray powder diffraction pattern further comprises peaks at 6.3°, 8.1°, and 12.6°±0.2 2θ as measured by X-ray powder diffraction using Cu K alpha radiation.

Embodiment K1. A composition comprising a solid form of Compound 10:

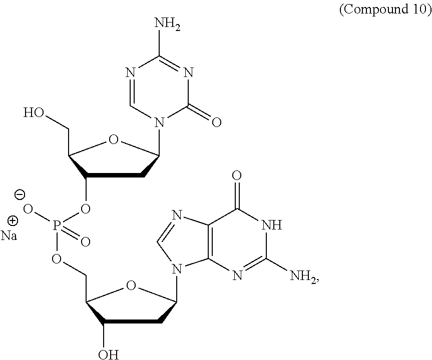 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

wherein the solid form exhibits an X-ray powder diffraction pattern substantially the same as the X-ray powder diffraction pattern shown in FIG. 8.

Embodiment L1. A composition comprising a solid form of Compound 10:

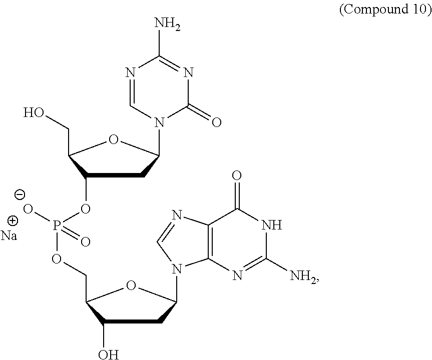 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

wherein the solid form has an X-ray powder diffraction pattern that comprises peaks at 9.3°, 10.8°, and 11.7±0.2 2θ as measured by X-ray powder diffraction using Cu K alpha radiation.

Embodiment L2. The composition of embodiment L1, wherein the X-ray powder diffraction pattern further comprises peaks at 13.3°, 13.9°, and 15.4°±0.2 2θ as measured by X-ray powder diffraction using Cu K alpha radiation.

Embodiment M1. A process for preparing Compound 10:

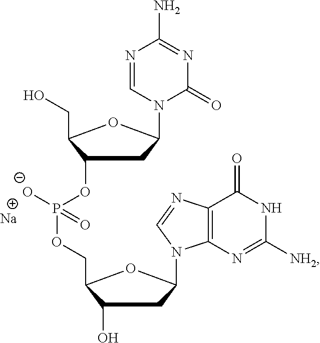 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

comprising contacting a first mixture with a sodium cation source to provide a second mixture, wherein the first mixture comprises Compound 9

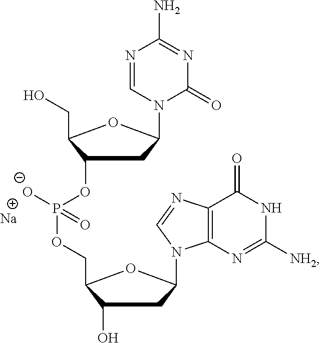 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

and dimethyl sulfoxide, and the second mixture comprises Compound 10.

Embodiment M2. The process of embodiment M1, wherein the sodium cation source is an aqueous sodium cation source.

Embodiment M3. The process of embodiment M1 or embodiment M2, wherein the sodium cation source comprises sodium acetate.

Embodiment M4. The process of any one of embodiments M1-M3, wherein the first mixture further comprises methanol.

Embodiment M5. The process of any one of embodiments M1-M4, further comprising isolating Compound 10 from the second mixture to provide a solid, wherein the solid comprises Compound 10.

Embodiment M6. The process of embodiment M5, wherein the isolating Compound 10 comprises contacting the second mixture with an antisolvent to provide a precipitate, and isolating the precipitate by filtration to provide the solid.

Embodiment M7. The process of embodiment M6, wherein the solid has a purity of at least about 96% (a/a) as determined by HPLC.

Embodiment M8. The process of embodiment M6, wherein the solid has a purity of at least about 99% (a/a) as determined by HPLC.

Embodiment M9. The process of embodiment M6, wherein the solid has a purity from about 96% to about 99.9% (a/a) as determined by HPLC.

Embodiment M10. The process of embodiment M6, wherein the solid has a purity from about 99% to about 99.9% (a/a) as determined by HPLC.

Embodiment M11. The process of embodiment M6, wherein the solid has a purity from about 99% to about 99.5% (a/a) as determined by HPLC.

Embodiment M12. The process of any one of embodiments M6-M11, wherein the antisolvent is a C₂-C₆ alcohol.

Embodiment M13. The process of any one of embodiments M6-M11, wherein the antisolvent comprises isopropanol.

Embodiment M14. The process of any one of embodiments M6-M11, wherein the antisolvent comprises ethanol.

Embodiment M15. The process of any one of embodiments M6-M11, wherein the contacting the second mixture with the antisolvent is conducted at a temperature no more than about 15° C.

Embodiment M16. The process of any one of embodiments M6-M14, wherein the contacting the second mixture with the antisolvent is conducted at a temperature from about −5° C. to about 15° C.

Embodiment M17. The process of any one of embodiments M6-M16, wherein the contacting the first mixture with the sodium cation source is conducted at a temperature no more than about 15° C.

Embodiment M18. The process of any one of embodiments M6-M16, wherein the contacting the first mixture with the sodium cation source is conducted at a temperature from about −5° C. to about 15° C.

Embodiment M19. The process of any one of embodiments M15-M18, wherein the solid is amorphous.

Embodiment M20. The process of any one of embodiments M6-M14, wherein the contacting the second mixture with the antisolvent is conducted at a temperature no more than about 35° C.

Embodiment M21. The process of any one of embodiments M6-M14, wherein the contacting the second mixture with the antisolvent is conducted at a temperature from about 15° C. to about 35° C.

Embodiment M22. The process of any one of embodiments M6-M14, M20, and M21, wherein the contacting the first mixture with the sodium cation source is conducted at a temperature no more than about 30° C.

Embodiment M23. The process of any one of embodiments M6-M14, M20, and M21, wherein the contacting the first mixture with the sodium cation source is conducted at a temperature from about 15° C. to about 30° C.

Embodiment M24. The process of any one of embodiments M20-M23, wherein the solid exhibits an X-ray powder diffraction pattern substantially the same as the X-ray powder diffraction pattern shown in FIG. 9.

Embodiment M25. The process of any one of embodiments M20-M23, wherein the solid exhibits an X-ray powder diffraction pattern that comprises peaks at 10°, 11.2°, and 12.2°±0.2 2θ as measured by X-ray powder diffraction using Cu K alpha radiation.

Embodiment M26. The process of embodiment M25, wherein the X-ray powder diffraction pattern further comprises peaks at 11.4°, 13.2°, and 14.3°±0.2 2θ as measured by X-ray powder diffraction using Cu K alpha radiation.

Embodiment M27. The process of embodiment M25 or embodiment M26, wherein the X-ray powder diffraction pattern further comprises peaks at 4.9°, 16.5°, and 18.4°±0.2 2θ as measured by X-ray powder diffraction using Cu K alpha radiation.

Embodiment N1. A process comprising:
(i) contacting a first mixture with ethanol to provide a second mixture, wherein the first mixture comprises water and Compound 10:

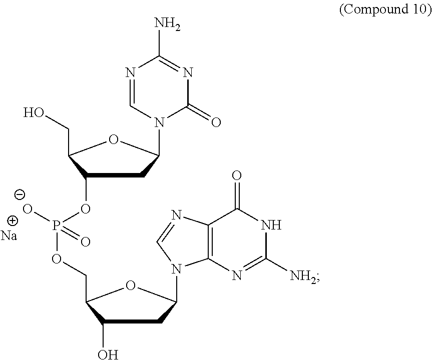 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

(ii) cooling the second mixture to provide a precipitate; and
(iii) isolating the precipitate via filtration to provide a polymorph of Compound 10.

Embodiment N2. The process of embodiment N1, wherein the polymorph has an X-ray powder diffraction pattern that comprises peaks at 4.9°, 7.2°, and 10.0°±0.2 2θ as measured by X-ray powder diffraction using Cu K alpha radiation.

Embodiment N3. The process of embodiment N2, wherein the X-ray powder diffraction pattern of the polymorph further comprises peaks at 11.3°, 11.5°, and 12.2°±0.2 2θ as measured by X-ray powder diffraction using Cu K alpha radiation.

Embodiment N4. The process of any one of embodiments N1-N3, further comprising washing the polymorph with a C₁-C₆ alcohol, wherein the C₁-C₆ alcohol is at a temperature of about 5° C. to about 15° C. during the washing.

Embodiment N4. The process of any one of embodiments N1-N3, further comprising washing the polymorph with ethanol that has a temperature of about 5° C. to about 15° C.

Embodiment N5. The process of any one of embodiments N1-N2, further comprising drying the polymorph under reduced pressure at a temperature from about 0° C. to about 30° C., and then at a temperature from about 30° C. to about 70° C. under reduced pressure to provide a solid comprising a second polymorph.

Embodiment N6. The process of embodiment N4, further comprising, after the washing, drying the polymorph under reduced pressure at a temperature from about 0° C. to about 30° C., and then at a temperature from about 30° C. to about 70° C. under reduced pressure to provide a solid comprising a second polymorph.

Embodiment N7. The process of any one of embodiments N1-N6, wherein the first mixture comprises from about 1% to about 10% (w/w) Compound 10.

Embodiment N8. The process of any one of embodiments N1-N6, wherein the first mixture comprises from about 3% to about 7% (w/w) Compound 10.

Embodiment N9. The process of any one of embodiments N1-N8, wherein the second mixture comprises from about 60% to about 90% (w/w) ethanol.

Embodiment N10. The process of any one of embodiments N1-N8, wherein the second mixture comprises from about 75% to about 80% (w/w) ethanol.

Embodiment N11. The process of any one of embodiments N1-N10, wherein the cooling the second mixture comprises cooling the second mixture to a temperature from about 5° C. to about 15° C.

Embodiment N12. The process of any one of embodiments N1-N11, wherein the contacting the first mixture is conducted at a temperature from about 5° C. to about 35° C.

Embodiment N13. The process of any one of embodiments N5-N12, wherein the second polymorph has an X-ray powder diffraction pattern that comprises peaks at 10.1°, 11.2°, and 14°±0.2 2θ as measured by X-ray powder diffraction using Cu K alpha radiation.

Embodiment N14. The process of embodiment N13, wherein the X-ray powder diffraction pattern of the second polymorph further comprises peaks at 15.3°, 17.7°, and 18.4°±0.2 2θ as measured by X-ray powder diffraction using Cu K alpha radiation.

Embodiment N15. The process of any one of embodiments N5-N14, wherein the reduced pressure is no more than 200 mbar.

Embodiment N16. The process of any one of embodiments N5-N14, wherein the reduced pressure is no more than 150 mbar.

Embodiment N17. The process of any one of embodiments N5-N14, wherein the reduced pressure is no more than 100 mbar.

Embodiment N18. The process of any one of embodiments N5, N6, and N13-N17, wherein the solid has a purity of at least about 96% (a/a) as determined by HPLC.

Embodiment N19. The process of any one of embodiments N5, N6, and N13-N17, wherein the solid has a purity of at least about 97% (a/a) as determined by HPLC.

Embodiment N20. The process of any one of embodiments N5, N6, and N13-N17, wherein the solid has a purity of at least about 98% (a/a) as determined by HPLC.

Embodiment N21. The process of any one of embodiments N5, N6, and N13-N17, wherein the solid has a purity of at least about 99% (a/a) as determined by HPLC.

Embodiment N22. The process of any one of embodiments N5, N6, and N13-N17, wherein the solid has a purity of at least about 99.5% (a/a) as determined by HPLC.

Embodiment N23. The process of any one of embodiments N5, N6, and N13-N17, wherein the solid has a purity from about 96% to about 99.9% (a/a) as determined by HPLC.

Embodiment N24. The process of any one of embodiments N5, N6, and N13-N17, wherein the solid has a purity from about 98% to about 99.9% (a/a) as determined by HPLC.

Embodiment O1. A process comprising:
(i) contacting Compound 10

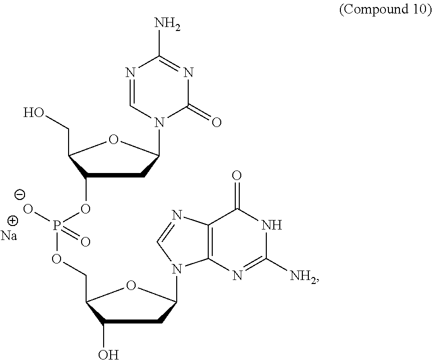 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

with a first mixture to provide a second mixture, wherein the first mixture comprises a solvent, wherein the solvent is a combination of water and ethanol;
(ii) heating the second mixture to a temperature of from about 30° C. to about 45° C.;
(iii) after the heating, cooling the second mixture to provide a precipitate; and
(iv) isolating the precipitate via filtration to provide a polymorph of Compound 10.

Embodiment O2. The process of embodiment O1, wherein the polymorph exhibits an X-ray powder diffraction pattern substantially the same as the X-ray powder diffraction pattern shown in FIG. 9.

Embodiment O3. The process of embodiment O1, wherein the polymorph exhibits an X-ray powder diffraction pattern that comprises peaks at 10°, 11.2°, and 12.2°±0.2 2θ as measured by X-ray powder diffraction using Cu K alpha radiation.

Embodiment O4. The process of embodiment O3, wherein the X-ray powder diffraction pattern of the polymorph further comprises peaks at 11.4°, 13.2°, and 14.3°±0.2 2θ as measured by X-ray powder diffraction using Cu K alpha radiation.

Embodiment O5. The process of embodiment O3, wherein the X-ray powder diffraction pattern of the polymorph further comprises peaks at 4.9°, 16.5°, and 18.4°±0.2 2θ as measured by X-ray powder diffraction using Cu K alpha radiation.

Embodiment O6. The process of any one of embodiments O1-O5, further comprising washing the polymorph with a C₁-C₆ alcohol, wherein the C₁-C₆ alcohol is at a temperature of about 5° C. to about 15° C. during the washing.

Embodiment O7. The process of any one of embodiments O1-O5, further comprising washing the polymorph with ethanol that has a temperature of about 5° C. to about 15° C.

Embodiment O8. The process of embodiment O7, further comprising, after the washing, drying the polymorph under reduced pressure at a temperature from about 0° C. to about 30° C., and then at a temperature from about 30° C. to about 50° C. under reduced pressure to provide a solid comprising a second polymorph.

Embodiment O9. The process of any one of embodiments O1-O7, further comprising drying the polymorph under reduced pressure at a temperature from about 0° C. to about 30° C., and then at a temperature from about 30° C. to about 50° C. under reduced pressure to provide a solid comprising a second polymorph.

Embodiment O10. The process of embodiment O8 or embodiment O9, wherein the second polymorph exhibits an X-ray powder diffraction pattern substantially the same as the X-ray powder diffraction pattern shown in FIG. 7.

Embodiment O11. The process of embodiment O8 or embodiment O9, wherein the second polymorph exhibits an X-ray powder diffraction pattern that comprises peaks at 5.1°, 10.2°, and 11.2°±0.2 2θ as measured by X-ray powder diffraction using Cu K alpha radiation.

Embodiment O12. The process of embodiment O11, wherein the X-ray powder diffraction pattern of the second polymorph further comprises peaks at 6.3°, 8.1°, and 12.6°±0.2 2θ as measured by X-ray powder diffraction using Cu K alpha radiation.

Embodiment O13. The process of embodiment O11, wherein the X-ray powder diffraction pattern of the second polymorph further comprises peaks at 12.6°, 14.3°, and 15.5°±0.2 2θ as measured by X-ray powder diffraction using Cu K alpha radiation.

Embodiment O14. The process of any one of embodiments O1-O13, wherein the solvent is from about 10% to about 30% (v/v) water in ethanol.

Embodiment O15. The process of any one of embodiments O1-O13, wherein the solvent is from about 15% to about 25% (v/v) water in ethanol.

Embodiment O16. The process of any one of embodiments O1-O15, wherein the cooling the second mixture comprises cooling the second mixture to a temperature from about 5° C. to about 15° C.

Embodiment O17. The process of any one of embodiments O1-O16, wherein the second mixture comprises from about 0.5% to about 10% (w/w) Compound 10.

Embodiment O18. The process of any one of embodiments O1-O16, wherein the second mixture comprises from about 0.5% to about 3% (w/w) Compound 10.

Embodiment O19. The process of any one of embodiments O8-O13, wherein the solid has a purity of at least about 96% (a/a) as determined by HPLC.

Embodiment O20. The process of any one of embodiments O8-O13, wherein the solid has a purity of at least about 97% (a/a) as determined by HPLC.

Embodiment O21. The process of any one of embodiments O8-O13, wherein the solid has a purity of at least about 98% (a/a) as determined by HPLC.

Embodiment O22. The process of any one of embodiments O8-O13, wherein the solid has a purity of at least about 99% (a/a) as determined by HPLC.

Embodiment O23. The process of any one of embodiments O8-O13, wherein the solid has a purity of at least about 99.5% (a/a) as determined by HPLC.

Embodiment O24. The process of any one of embodiments O8-O13, wherein the solid has a purity from about 96% to about 99.9% (a/a) as determined by HPLC.

Embodiment O25. The process of any one of embodiments O8-O13, wherein the solid has a purity from about 98% to about 99.9% (a/a) as determined by HPLC.

Embodiment P1. A process for preparing Compound 10:

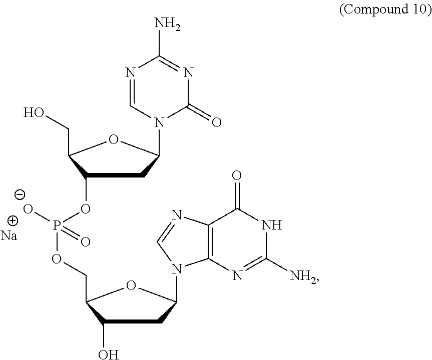 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]

comprising:
(a) removing the 2-cyanoethyl and N,N-dimethylformamidine groups of Compound 7:

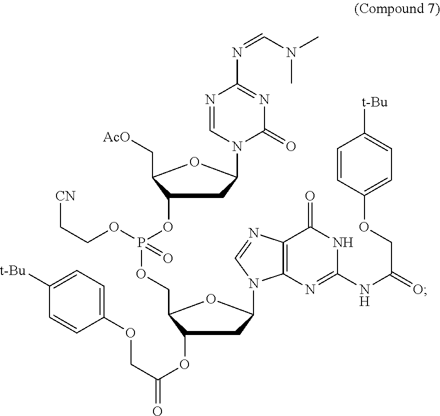 [SMILES: CC(=O)OC[C@H]1O[C@@H](n2cnc(/N=C\N(C)C)nc2=O)C[C@H]1OP(=O)(OCCC#N)OC[C@H]1O[C@@H](n2cnc3c(=O)[nH]c(NC(=O)COc4ccc(C(C)(C)C)cc4)nc32)C[C@H]1OC(=O)COc1ccc(C(C)(C)C)cc1]

with tert-butylamine to form Compound 8:

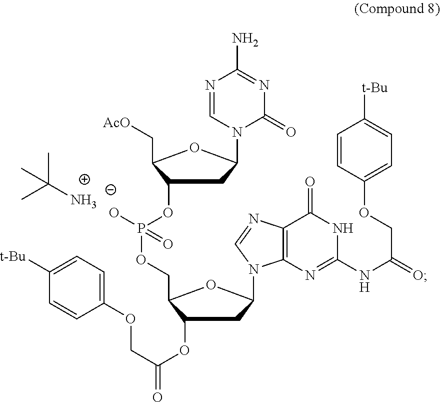

and
(b) removing the acetyl and (4-tertbuylphenoxy)acetyl groups of Compound 8 to form a deprotected adduct, and protonating the deprotected adduct to form Compound 9:

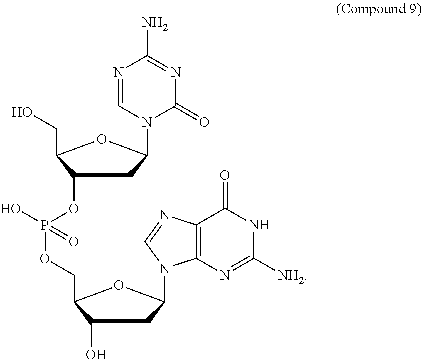 [SMILES: Nc1ncn([C@H]2C[C@@H](OP(=O)(O)OC[C@H]3O[C@@H](n4cnc5c(=O)[nH]c(N)nc54)C[C@H]3O)[C@@H](CO)O2)c(=O)n1]

Embodiment P2. The process of embodiment P1, wherein the removing the 2-cyanoethyl and N,N-dimethylformamidine groups of Compound 7 comprises contacting the tert-butylamine with a mixture, wherein the mixture comprises Compound 7 and a polar organic solvent.

Embodiment P3. The process of embodiment P1, wherein the removing the 2-cyanoethyl and N,N-dimethylformamidine groups of Compound 7 comprises contacting the tert-butylamine with a mixture, wherein the mixture comprises Compound 7 and acetonitrile.

Embodiment P4. The process of embodiment P2 or embodiment P3, wherein the mixture further comprises tetrahydrofuran.

Embodiment P5. The process of any one of embodiments P1-P4, wherein the removing the acetyl and (4-tertbuylphenoxy)acetyl groups of Compound 8 comprises contacting Compound 8 with a base.

Embodiment P6. The process of embodiment P5, wherein the base is an alkoxide.

Embodiment P7. The process of embodiment P5, wherein the base is sodium methoxide.

Embodiment P8. The process of any one of embodiments P1-P7, wherein the removing the acetyl and (4-tertbuylphenoxy)acetyl groups of Compound 8 comprises contacting a base with a mixture, wherein the mixture comprises Compound 8 and a polar organic solvent.

Embodiment P9. The process of embodiment P8, wherein the base is an alkoxide.

Embodiment P10. The process of embodiment P8, wherein the base is sodium methoxide.

Embodiment P11. The process of any one of embodiments P8-P10, wherein the polar organic solvent comprises methanol.

Embodiment P12. The process of any one of embodiments P1-P11, wherein the protonating the deprotected adduct to form Compound 9 comprises contacting the deprotected adduct with an acid.

Embodiment P13. The process of embodiment P12, wherein the acid is acetic acid.

Embodiment P14. The process of any one of embodiments P1-P13, further comprising contacting Compound 9 with a sodium cation source to form Compound 10.

Embodiment P15. The process of embodiment P14, wherein the contacting Compound 9 with the sodium cation source to form Compound 10 comprises contacting the sodium cation source with a second mixture, wherein the second mixture comprises Compound 9 and a polar organic solvent.

Embodiment P16. The process of embodiment P14, wherein the contacting Compound 9 with a sodium cation source to form Compound 10 comprises contacting the sodium cation source with a second mixture, wherein the second mixture comprises Compound 9 and methanol.

Embodiment P17. The process of embodiment P15 or embodiment P16, wherein the second mixture further comprises dimethyl sulfoxide.

Embodiment P18. The process of any one of embodiments P14-P17, wherein the sodium cation source comprises a sodium carboxylate or sodium alkoxide.

Embodiment P19. The process of any one of embodiments P14-P17, wherein the sodium cation source comprises sodium acetate.

Embodiment P20. The process of any one of embodiments P14-P17, wherein the sodium cation source is aqueous sodium acetate.

Embodiment P21. The process of any one of embodiments P14-P17, wherein the contacting Compound 9 with a sodium cation source to form Compound 10 is conducted at no more than about 15° C.

Embodiment P22. The process of any one of embodiments P14-P17, wherein the contacting Compound 9 with a sodium cation source to form Compound 10 is conducted at from about −5° C. to about 15° C.

What is claimed is:

1. A compound of formula (IVa): 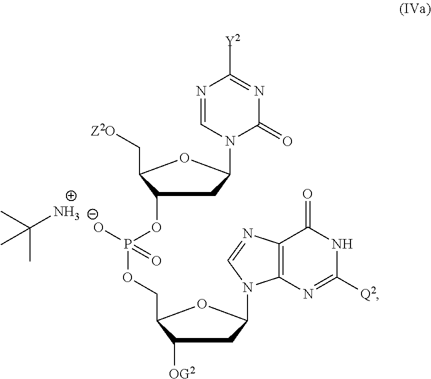
wherein:
each Z² and G² is independently H or a protecting group; and
each Y² and Q² is independently NH₂ or a protected primary amine.

2. The compound of claim 1, wherein each Z² and G² is independently H, substituted or unsubstituted acetyl, substituted or unsubstituted phenoxyacetyl, substituted or unsubstituted ethoxymethyl, substituted or unsubstituted benzoyl, or a silyl protecting group.

3. The compound of claim 1, wherein each Y² and Q² is independently NH₂, or a primary amine protected with substituted or unsubstituted acetyl, substituted or unsubstituted phenoxyacetyl, substituted or unsubstituted ethoxymethyl, substituted or unsubstituted benzoyl, a silyl protecting group, or a formamidine group.

4. The compound of claim 1, wherein each Z² and G² is independently H, benzoyl, acetyl, isobutyryl, Pac, Tac, iPr-Pac, [(triisopropylsilyl)oxy]methyl, tert-butyldimethylsilyl, or 2′-cyanoethoxymethyl.

5. The compound of claim 1, wherein each Y² and Q² is independently NH₂, or a primary amine protected with benzoyl, acetyl, isobutyryl, Pac, Tac, iPr-Pac, [(triisopropylsilyl)oxy]methyl, tert-butyldimethylsilyl, or 2′-cyanoethoxymethyl.

6. The compound of claim 1, wherein each Z² and G² is independently H, acetyl, Pac, Tac, or iPr-Pac.

7. The compound of claim 1, wherein each Y² and Q² is independently NH₂, or a primary amine protected with acetyl, Pac, Tac, iPr-Pac.

8. The compound of claim 1, wherein Z² is H or acetyl.

9. The compound of claim 1, wherein G² is H or Tac.

10. The compound of claim 1, wherein Y² is NH₂ or NH(Tac).

11. The compound of claim 1, wherein Q² is NH₂ or NH(Tac).

12. The compound of claim 1, wherein the compound is:

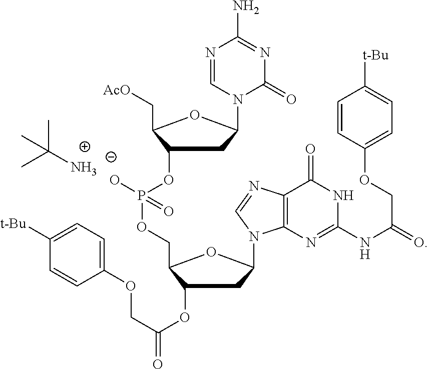

13. A process for producing a polymorph of Compound 10: 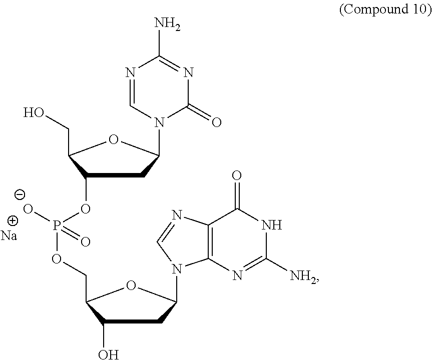 [SMILES: [Na+] ; Nc1ncn([C@H]2C[C@@H](OP(=O)([O-])OC[C@H]3O[C@@H](n4cnc5c(=O)[nH]c(N)nc54)C[C@H]3O)[C@@H](CO)O2)c(=O)n1]
comprising drying under reduced pressure a solid form of Compound 10 to provide the polymorph, wherein the solid form of Compound 10 has an X-ray powder diffraction pattern that comprises peaks at 4.9°, 7.2°, and 10.0°±0.2 2θ as measured by X-ray powder diffraction using Cu K alpha radiation.

14. The process of claim 13, wherein the X-ray powder diffraction pattern of the solid form further comprises peaks at 11.3°, 11.5°, and 12.2°±0.2 2θ as measured by X-ray powder diffraction using Cu K alpha radiation.

15. The process of claim 13, wherein the polymorph has an X-ray powder diffraction pattern that comprises peaks at 10.1°, 11.2°, and 14°±0.2 2θ as measured by X-ray powder diffraction using Cu K alpha radiation.

16. The process of claim 15, wherein the X-ray powder diffraction pattern of the polymorph further comprises peaks at 15.3°, 17.7°, and 18.4°±0.2 2θ as measured by X-ray powder diffraction using Cu K alpha radiation.

17. The process of claim 13, wherein the reduced pressure is no more than 200 mbar.

18. The process of claim 13, wherein the reduced pressure is no more than 150 mbar.

19. The process of claim 13, wherein the reduced pressure is no more than 100 mbar.

20. The process of claim 13, wherein the drying under reduced pressure further comprises heating at a temperature of at least 40° C.
